(19) United States
(12) Patent Application Publication
(10) Pub. No.: US 2023/0192821 A1
(43) Pub. Date: Jun. 22, 2023

(54) POLYPEPTIDES ENCODING ANTIBODIES BINDING TO SARS-COV-2 SPIKE PROTEIN

(71) Applicants: Alchemab Therapeutics Ltd, London (GB); Barts Health NHS Trust, London (GB)

(72) Inventors: Jane Osbourn, London (GB); Ralph Minter, London (GB); Jacob Galson, London (GB)

(73) Assignees: Alchemab Therapeutics Ltd, London (GB); BARTS HEALTH NHS TRUST, London (GB)

(21) Appl. No.: 17/926,549

(22) PCT Filed: May 20, 2021
(86) PCT No.: PCT/GB2021/051221

§ 371 Date: Nov. 18, 2022

(30) Foreign Application Priority Data
May 20, 2020 (GB) 2007532.1

(51) Int. Cl. C07K 16/10; A61K 45/06; A61K 39/42; A61P 11/00; A61P 31/14

(52) U.S. Cl. CPC C07K 16/10; A61K 45/06; A61K 39/42; A61P 11/00; A61P 31/14; A61K 2039/505

(57) ABSTRACT

There is provided inter alia a polypeptide comprising a CDRH1 sequence comprising or consisting of a sequence sharing 80% or greater sequence identity with a CDRH1 sequence as shown in Table 1 and/or a CDRH2 sequence comprising or consisting of a sequence sharing 80% or greater sequence identity with a CDRH2 sequence as shown in Table 1 and/or a CDRH3 sequence comprising or consisting of a sequence sharing 80% or greater sequence identity with a CDRH3 sequence as shown in Table 1.

### CROSS REFERENCE TO RELATED APPLICATIONS

This application claims priority to UK Patent Application Number 2007532.1 filed on May 20, 2020 entitled POLYPEPTIDES, the contents of which are herein incorporated by reference in their entirety.

### SEQUENCE LISTING

The present application is being filed along with a Sequence Listing in electronic format. The Sequence Listing file, entitled 2231_1000PCT.txt, was created on May 19, 2021 and is 1,327,961 bytes in size. The information in electronic format of the Sequence Listing is incorporated herein by reference in its entirety.

### FIELD OF THE INVENTION

The present invention relates to polypeptides which were identified in the BCR heavy chain repertoire of individuals during SARS-CoV-2 infection. The invention also includes polynucleotides encoding said polypeptides, pharmaceutical compositions comprising said polypeptides and the use of said polypeptides in suppressing or treating a disease or disorder mediated by infection with SARS-CoV-2, for providing prophylaxis to a subject at risk of infection of SARS-CoV-2 or for the diagnosis and/or prediction of outcome of SARS-CoV-2 infection.

### BACKGROUND OF THE INVENTION

Since the report of the first patients in December 2019^{1,2}, the unprecedented global scale of the COVID-19 pandemic has become apparent. The infectious agent, the SARS-CoV-2 betacoronavirus³, causes mild symptoms in most cases but can cause severe respiratory diseases such as acute respiratory distress syndrome in some individuals. Risk factors for severe disease include age, male gender and underlying co-morbidities⁴.

Understanding the immune response to SARS-CoV-2 infection is critical to support the development of therapies. Recombinant monoclonal antibodies derived from analysis of B cell receptor (BCR) repertoires in infected patients or the immunisation of animals have been shown to be effective against several infectious diseases including Ebola virus⁵, rabies⁶ and respiratory syncytial virus disease⁷. Such therapeutic antibodies have the potential to protect susceptible populations as well as to treat severe established infections.

While many vaccine approaches are underway in response to the SARS-CoV-2 outbreak, many of these compositions include as immunogens either whole, attenuated virus or whole spike (S) protein—a viral membrane glycoprotein which mediates cell uptake by binding to host angiotensin-converting enzyme 2 (ACE2). The antibody response to such vaccines will be polyclonal in nature and will likely include both neutralising and non-neutralising antibodies. It is hoped that the neutralising component will be sufficient to provide long-term SARS-CoV-2 immunity following vaccination, although other potential confounders may exist, such as raising antibodies which mediate antibody-dependent enhancement (ADE) of viral entry⁸⁻¹⁰. While ADE is not proven for SARS-CoV-2, prior studies of SARS-CoV-1 in non-human primates showed that, while some S protein antibodies from human SARS-CoV-1 patients were protective, others enhanced the infection via ADE¹¹. An alternative could be to support passive immunity to SARS-CoV-2, by administering one, or a small cocktail of, well-characterised, neutralising antibodies.

Patients recovering from COVID-19 have already been screened to identify neutralising antibodies, following analysis of relatively small numbers (100-500) of antibody sequences^{12,13}. A more extensive BCR repertoire analysis was performed on six patients in Stanford, USA with signs and symptoms of COVID-19 who also tested positive for SARS-CoV-2 RNA¹⁴. Although no information was provided on the patient outcomes in that study, the analysis demonstrated preferential expression of a subset of immunoglobulin heavy chain (IGH) V gene segments with relatively little somatic hypermutation and showed evidence of convergent antibodies between patients.

To drive a deeper understanding of the nature of humoral immunity to SARS-CoV-2 infection and to identify potential therapeutic antibodies to SARS-CoV-2, we have evaluated the BCR heavy chain repertoire from 19 individuals at various stages of their immune response. We show that (1) there are stereotypic responses to SARS-CoV-2 infection, (2) infection stimulates both naïve and memory B cell responses, (3) sequence convergence can be used to identify putative SARS-CoV-2 specific antibodies, and (4) sequence convergence can be identified between different SARS-CoV-2 studies in different locations and using different sample types.

Polypeptides of the present invention may, in at least some embodiments, have one or more of the following advantages compared to the prior art:

(i) increased binding affinity to SARS-CoV-2, for example SARS-CoV-2 spike protein,

(ii) increased neutralising potency against SARS-CoV-2,

(iii) binding to non-spike protein components of SARS-CoV-2 to reduce viral load,

(iv) binding to host proteins to inhibit virus entry/infection,

(v) binding to SARS-CoV-2 infected human cells to enable infected cell killing,

(vi) binding to human cells or soluble factor to modulate immune response to the virus,

(vii) binding to human cells to alter innate immune responses from structural cells such as epithelial cells,

(viii) binding to endothelial cells to alter viral-related endothelial inflammation and modulation of the clotting response,

(ix) activity across all potential anti-viral mechanisms including novel ones (e.g., binding viral epitopes, secreted host epitopes, membrane host epitopes, modulating infected host cells, modulating innate and adaptive immune responses)

(x) neutralising potential against other/new forms of coronavirus,

(xi) suitability for administration with other agents in treating COVID-19 (e.g., to enhance anti-viral efficacy), (xii) suitable for prevention or treatment of SARS-CoV-2 infection,

(xii) suitability for administration by multiple routes (SC, IV, IM, dermal, nasal, oral),

(xiii) one or more polypeptides can be used in the diagnosis or prediction of outcome post SARS-CoV-2 infection.

### SUMMARY OF THE INVENTION

According to a first aspect of the invention, there is provided a polypeptide comprising:

a CDRH1 sequence comprising or consisting of a sequence sharing 80% or greater sequence identity with a CDRH1 sequence as shown in Table 1 and/or

a CDRH2 sequence comprising or consisting of a sequence sharing 80% or greater sequence identity with a CDRH2 sequence as shown in Table 1 and/or

a CDRH3 sequence comprising or consisting of a sequence sharing 80% or greater sequence identity with a CDRH3 sequence as shown in Table 1.

In a further aspect there is provided a polypeptide comprising:

a FWRH1 sequence comprising or consisting of a sequence sharing 80% or greater sequence identity with a FWRH1 sequence as shown in Table 1 and/or

a FWRH2 sequence comprising or consisting of a sequence sharing 80% or greater sequence identity with a FWRH2 sequence as shown in Table 1 and/or

a FWRH3 sequence comprising or consisting of a sequence sharing 80% or greater sequence identity with a FWRH3 sequence as shown in Table 1 and/or

a FWRH4 sequence comprising or consisting of a sequence sharing 80% or greater sequence identity with a FWRH4 sequence as shown in Table 1.

In a further aspect there is provided pharmaceutical compositions comprising the polypeptides above and polynucleotides encoding the polypeptides above. Further aspects of the invention will be apparent from the detailed description of the invention.

### DESCRIPTION OF THE FIGURES

FIG. 1A. B cell responses to SARS-COV-2 infection. IGHV gene segment usage distribution per isotype subclass. Bars show mean values+/− standard error of the mean. Comparisons performed using t-tests, with adjusted p values using Bonferroni correction for multiple comparisons; *p<0.05, **p<0.005, ***p<0.0005.

FIG. 1B. B cell responses to SARS-COV-2 infection. Isotype subclass distribution between IGHA and IGHG subclasses. Bars show mean values+/− standard error of the mean. Comparisons performed using t-tests, with adjusted p values using Bonferroni correction for multiple comparisons; *p<0.05, **p<0.005, ***p<0.0005.

FIG. 1C. B cell responses to SARS-COV-2 infection. Mean BCR CDRH3 lengths from COVID-19 patients compared to healthy controls. Bars show mean values+/− standard error of the mean. Comparisons performed using t-tests, with adjusted p values using Bonferroni correction for multiple comparisons; *p<0.05, **p<0.005, ***p<0.0005.

FIG. 2A. Response characteristics of SARS-CoV-2 infection. Distribution of sequences with different numbers of mutations from germline.

FIG. 2B. Response characteristics of SARS-CoV-2 infection. Relationship between the proportion of the repertoire comprised by unmutated sequences, and the disease state.

FIG. 2C. Response characteristics of SARS-CoV-2 infection. Individual sequences were clustered together into related groups to identify clonal expansions (clonotypes). Diversity of all clonotypes in the repertoire calculated using the Shannon diversity index. To normalise for different sequence numbers for each sample, a random subsample of 1,000 sequences was taken.

FIG. 2D. Response characteristics of SARS-CoV-2 infection. Correlation between the Shannon diversity index, and the proportion of unmutated sequences.

FIG. 2E. Response characteristics of SARS-CoV-2 infection. The percent of all sequences that fall into the largest 10 clonotypes.

FIG. 2F. Response characteristics of SARS-CoV-2 infection. Mean number of mutations of all sequences in the largest 10 clonotypes.

FIG. 3A. Convergent BCR sequence signature within individuals infected with SARS-CoV-2. Data from all patients and healthy controls were clustered together to identify convergent clonotypes. Shown is the number of clonotypes shared by different numbers of participants, grouped by whether the clonotypes are also present in the healthy control dataset.

FIG. 3B. Convergent BCR sequence signature within individuals infected with SARS-CoV-2. Data from all patients and healthy controls were clustered together to identify convergent clonotypes. Of the convergent clonotypes, the mean mutation count was compared between those that were convergent only within the SARS-CoV-2 patients, and those that were also convergent with the healthy control dataset.

FIG. 3C. Convergent BCR sequence signature within individuals infected with SARS-CoV-2. Data from all patients and healthy controls were clustered together to identify convergent clonotypes. Of the convergent clonotypes, the CDRH3 AA sequence length was compared between those that were convergent only within the SARS-CoV-2 patients, and those that were also convergent with the healthy control dataset.

FIG. 3D. Convergent BCR sequence signature within individuals infected with SARS-CoV-2. Data from all patients and healthy controls were clustered together to identify convergent clonotypes. Shown is a heatmap of the 777 convergent COVID-19-associated clonotypes (observed between 4 or more COVID-19 participants) with the 469 convergent clonotypes from seven metastatic breast cancer (BC) patient biopsy samples, demonstrating that the convergent signatures are unique to each disease cohort.

FIG. 3E. Convergent BCR sequence signature within individuals infected with SARS-CoV-2. Data from all patients and healthy controls were clustered together to identify convergent clonotypes. Shown is the percentage frequencies of four example convergent clonotypes grouped by clinical status. Disclosed are SEQ ID NOS 570, 468, 435, and 467, respectively, in order of appearance

FIG. 3F. Convergent BCR sequence signature within individuals infected with SARS-CoV-2. Data from all patients and healthy controls were clustered together to identify convergent clonotypes. Shown is a similarity tree of convergent clonotype cluster centers that are significantly associated with clinical status. Groups (i) and (ii) indicate groups of similar convergent clonotypes. An alignment of group (ii) provided adjacent. Disclosed are SEQ ID NOS 907, 943, 433, 570, 461, 435, 974, 655, 468, 481, 552, 480, 458, 467, 722, 742, 851, 558, 440, 540, 463, 487, 575, 559, 416, 467, 722, 742, 851, and 558, respectively, in order of columns.

FIG. 3G. Convergent BCR sequence signature within individuals infected with SARS-CoV-2. Data from all patients and healthy controls were clustered together to identify convergent clonotypes. Proportions of IGHA and IGHG of the convergent clonotypes that are associated with patients with improving symptoms are shown.

FIG. 4A. Matches of the 777 convergent clonotypes identified in the present study to other SARS-CoV-2 studies. CDRH3 sequence (shown across the top in black text, SEQ ID NO: 2002), and IGHV/IGHJ gene segments of a sequence identified in the bronchoalveolar lavage fluid of a SARS-CoV-2 patient from a Chinese cohort, and a CDRH3 AA sequence logo unpacking the sequence diversity present in the convergent clonotype found in the COVID-19 patients in this study that had an exact AA match.

FIG. 4B. Matches of the 777 convergent clonotypes identified in the present study to other SARS-CoV-2 studies. CDRH3 sequence (shown across the top in black text, SEQ ID NO: 2015), and IGHV/IGHJ gene segment of an antibody in the CoV-AbDab (S304) that has SARS-CoV-1 and SARS-CoV-2 neutralising activity, alongside a CDRH3 AA sequence logo unpacking the sequence diversity in the convergent clonotype found in the COVID-19 patients in this study that had an exact AA match.

FIG. 4C. Matches of the 777 convergent clonotypes identified in the present study to other SARS-CoV-2 studies. Shown is a comparison of convergent clonotypes to the BCR data from Nielsen et al¹⁴. Plotted along the x-axis are the 405 convergent clonotypes represented in at least one Nielsen et al. dataset. Each row represents a separate BCR repertoire from Nielsen et al.; Non-shaded area indicates that the convergent clonotype has a match in the Nielsen dataset.

FIG. 5A. Distribution of sequences with different numbers of mutations from germline. Each row is a different COVID-19 patient (right).

FIG. 5B. Distribution of sequences with different numbers of mutations from germline. Each row is a different COVID-19 patient (right).

FIG. 5C. Distribution of sequences with different numbers of mutations from germline. Each row is a different COVID-19 patient (right).

FIG. 5D. Distribution of sequences with different numbers of mutations from germline. Each row is a different COVID-19 patient (right).

FIG. 6. The proportion of IGHG1 sequences containing the autoreactive “NHS” and “AVY” motifs between COVID patients with improving, stable or worsening symptoms. IGHG1 (box) was the only significant correlation. P-values are determined by ANOVA.

FIG. 7A. Properties of the 777 convergent clonotypes. Pie chart shows isotype subclass usage of the sequences with the 777 convergent clonotypes.

FIG. 7B. Properties of the 777 convergent clonotypes. Graph shows IGHV gene segment usage of the 777 convergent clonotypes.

FIG. 8A. Percentage frequencies of the convergent clonotypes grouped by clinical status that significantly associated with clinical status. Disclosed are SEQ ID NOS 655, 943, 552, 559, 575, 463, 742, 570, 435, 416, 481, and 468, respectively, in order of appearance.

FIG. 8B. Percentage frequencies of the convergent clonotypes grouped by clinical status that significantly associated with clinical status. Disclosed are SEQ ID NOS 487, 722, 461, 467, 540, 558, 480, 458, 440, 974, 851, 433, and 907, respectively, in order of appearance.

FIG. 9A. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

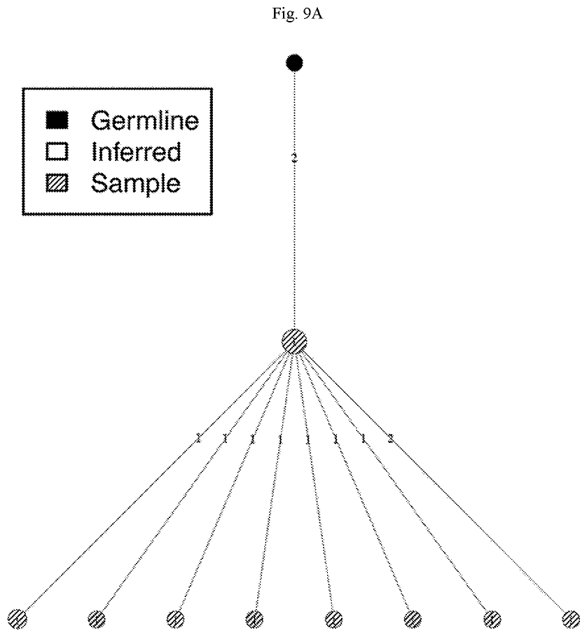

FIG. 9B. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

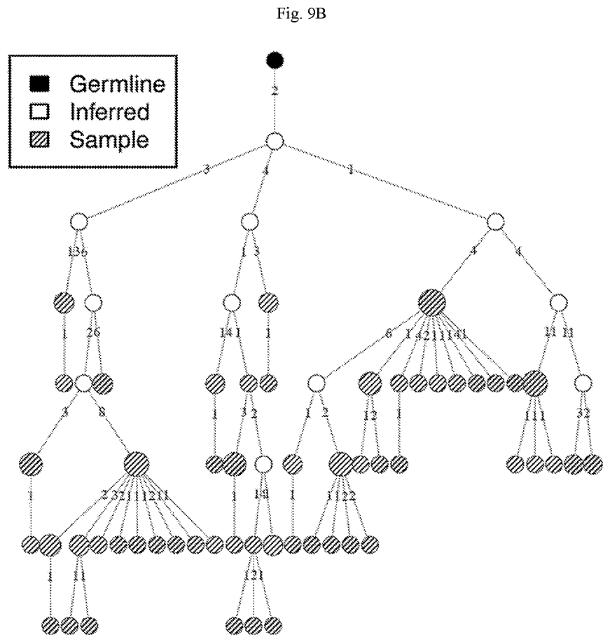

FIG. 9C. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

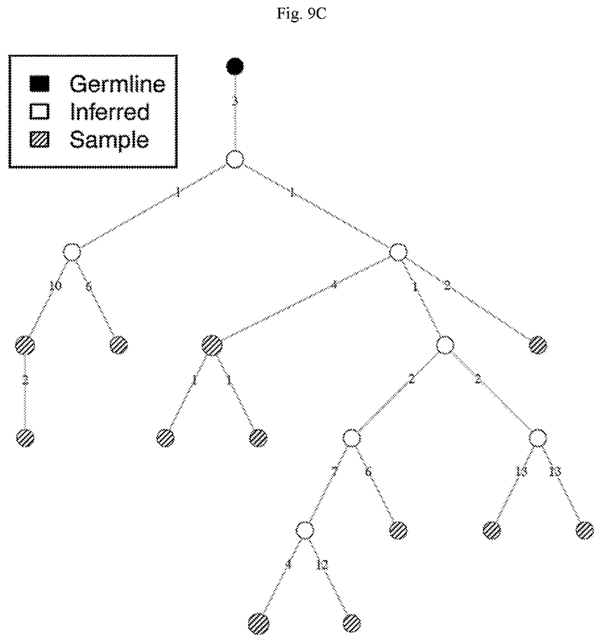

FIG. 9D. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

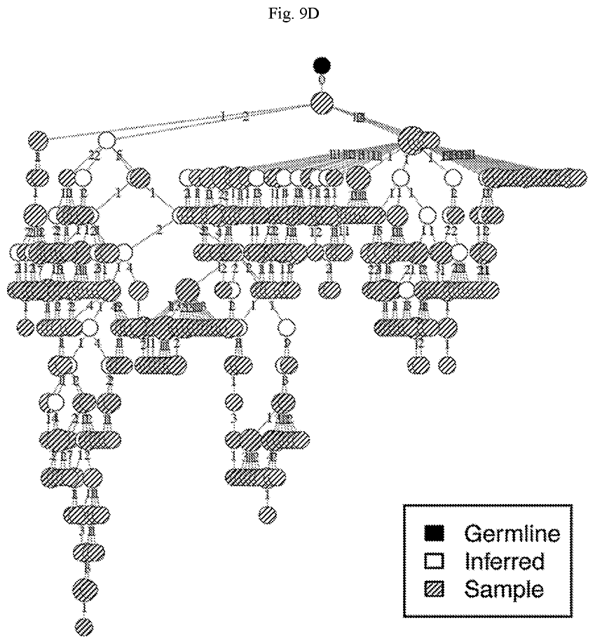

FIG. 9E. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

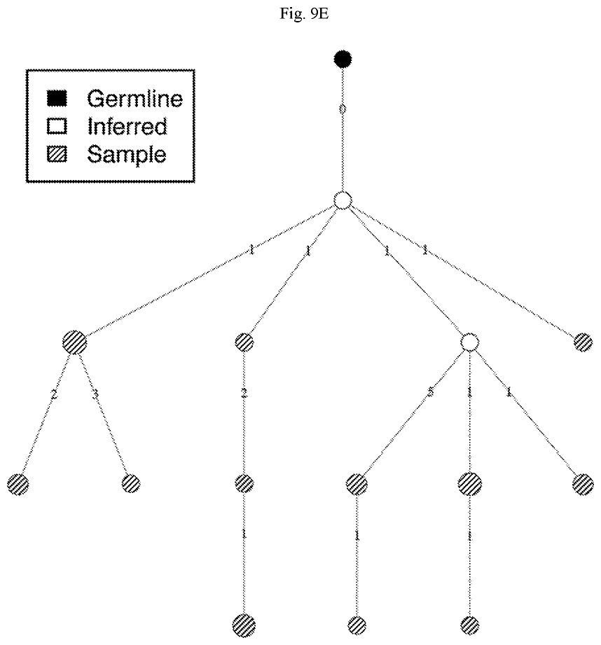

FIG. 9F. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

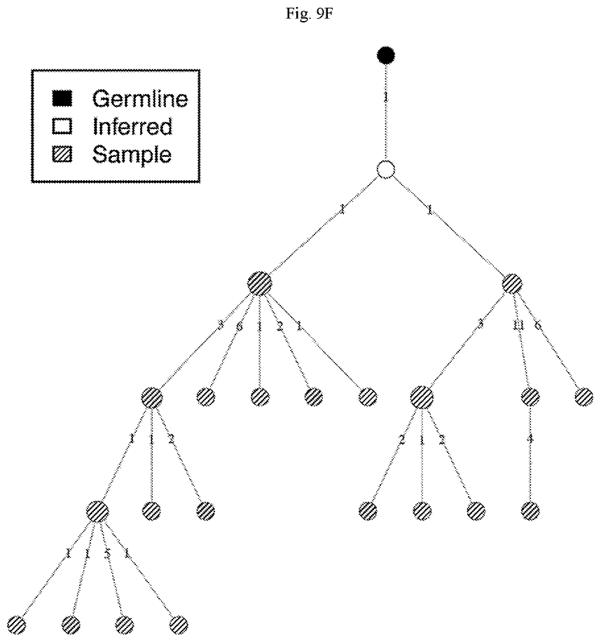

FIG. 9G. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

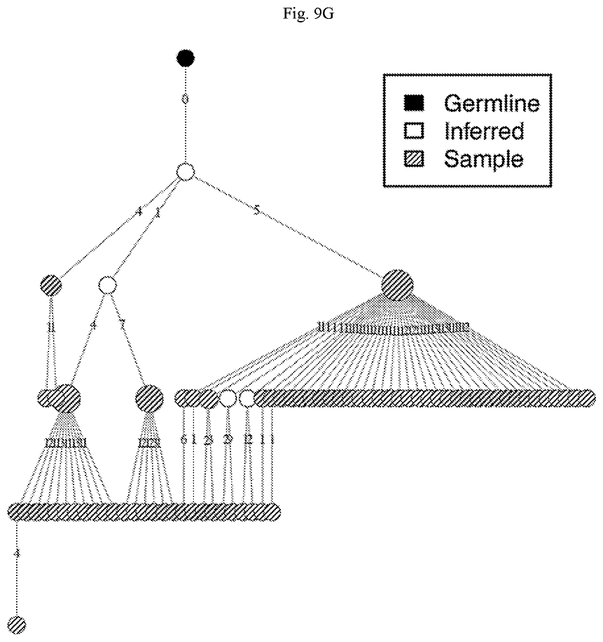

FIG. 9H. Lineage tree of the convergent clonotype that matched to the bronchoalveolar lavage fluid data. Lineage tree represents the members of the clonotype from the patient it was present in. Each node represents a unique sequence within the clonotype lineage tree, with the size indicative of the number of duplicate sequences present. Numbers on the edges of adjoining nodes show the number of mutations between the sequences.

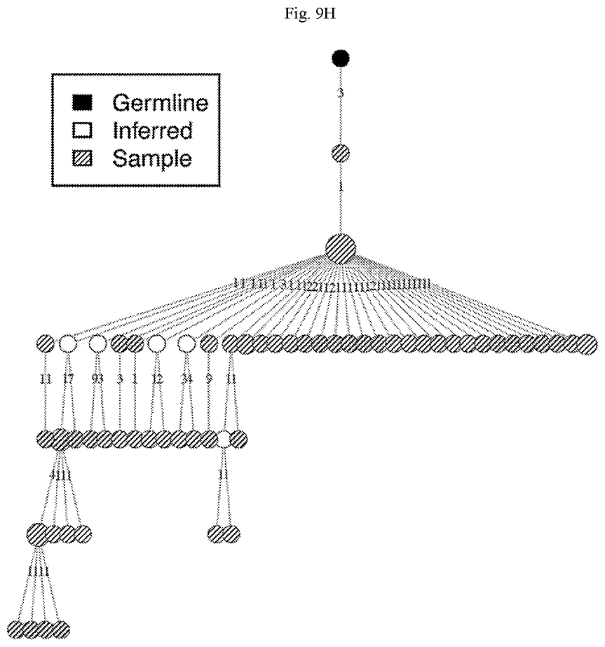

FIG. 10. Logo plots unpacking the sequence diversity present for the convergent clonotypes that clustered with CoV-AbDab SARS-CoV-1 or SARS-CoV-2 binding antibodies. The CoV-AbDab reference CDRH3 (corresponding to SEQ ID NOS 2015-2020, respectively, in order of appearance) and IGHV/IGHJ gene segment is displayed above each Logo plot. Gene transcript matches are annotated with “*,” while mismatches are annotated with “**.” The full sequence for 31B9 is not yet publicly available, so its genetic origins are not determined (ND).

### DETAILED DESCRIPTION

The complementarity determining regions (CDRs) and framework regions (FWRs) of an antibody or fragment thereof may be numbered from N- to C-terminus, i.e. FWR1, CDR1, FWR2, CDR2, FWR3, CDR3 and FWR4. In the context of a heavy chain variable domain, these regions may be denoted with an ‘H’, i.e. FWRH1, CDRH1, FWRH2, CDRH2, FWRH3, CDRH3 and FWRH4.

Table 1 below provides the polypeptide sequences of immunoglobulin heavy chain variable domains of the invention (VHs) with complementarity determining regions (CDRH1-3) and frameworks (FWRH1-4) of the invention annotated according to the IMGT system (Lefranc et al. “IMGT unique numbering for immunoglobulin and T cell receptor variable domains and Ig superfamily V-like domains” Dev. Comp. Immunol. 27(1):55-77 (2003)). The full length polypeptide sequence of any VH given in Table 1 is the combination of, from N- to C-terminus, FWRH1, CDRH1, FWRH2, CDRH2, FWRH3, CDRH3 and FWRH4 on a single row. For example, the polypeptide sequence of set1_1 is QVQLVESGGGVVQPGRSLRLSCAASGFTFSSYAMHWVRQAPGKGLEWVAVISYDG SNKYYADSVKGRFTISRDNSKNTLYLQMNSLRAEDAVYYCARDGGGYMDVWGQG TTVTVSS (SEQ ID NO: 1). “v_call” and “j_call” refer to the germline V and J gene segments from which the sequence originated, according to the IMGT system.

Table 2 below also provides the polypeptide sequences of immunoglobulin heavy chain variable domains (VHs) of the invention.

Based on the experimental work provided herein, it is expected that components of these VHs, such as the complementarity determining regions, frameworks, or combinations of these (such as full length VH sequences) may be utilised in therapeutic or prophylactic agents for treating or preventing SARS-CoV-2 infection, or for performing diagnostic or prognostic analysis of subjects infected, or suspected of being infected, with SARS-CoV-2.

It is envisaged that the proposed heavy chains be paired with suitable light chains to enable production of monoclonal antibodies, for example in IgG1 format. Cognate light chains can be identified by various methods, including computational prediction (eg Mason et al bioRxiv 617860 (2019)), the use of promiscuous or ‘common light chains’ (eg Xue et al. Biochem Biophys Res Commun. 515(3):481-486, (2019)), high-throughput paired heavy and light chain sequencing to identify native pairings (eg Wang et al Nat Biotechnol. 36(2):152-155 (2018)) and antibody display-based methods to find and optimise heavy and light chain pairings (eg Guo-Qiang et al. Methods Mol Biol. 562:133-142 2009).

TABLE 1
Polypeptide sequences of immunoglobulin heavy chain variable domains
(VHs), from N-to C-terminus, with frameworks and complementarity determining
regions annotated according to the IMGT system

| | | | FWRH1 | CDRH1 | FWRH2 | CDRH2 | FWRH3 | CDRH3 | FWRH4 |
|---|---|---|---|---|---|---|---|---|---|
| VH | | | (SEQ ID | (SEQ ID | (SEQ ID | (SEQ ID | (SEQ ID | (SEQ ID | (SEQ ID |
| name | v_call | j_call | NO) | NO) | NO) | NO) | NO) | NO) | NO) |
| set1_1 | IGHV3- | IGHJ6 | 2 | 38 | 135 | 216 | 304 | 416 | 1196 |
| | 30-3 | | | | | | | | |
| set1_2 | IGHV3- | IGHJ4 | 2 | 39 | 135 | 216 | 304 | 417 | 1197 |
| | 30 | | | | | | | | |
| set1_3 | IGHV4- | IGHJ5 | 3 | 40 | 136 | 217 | 305 | 418 | 1197 |
| | 59 | | | | | | | | |
| set1_4 | IGHV3- | IGHJ4 | 2 | 39 | 135 | 218 | 304 | 419 | 1197 |
| | 33 | | | | | | | | |
| set1_5 | IGHV1- | IGHJ3 | 4 | 41 | 137 | 219 | 306 | 420 | 1197 |
| | 69 | | | | | | | | |
| set2_1 | IGHV3- | IGHJ6 | 5 | 42 | 138 | 220 | 307 | 421 | 1196 |
| | 73 | | | | | | | | |
| set2_2 | IGHV3- | IGHJ4 | 6 | 43 | 139 | 221 | 308 | 422 | 1197 |
| | 74 | | | | | | | | |
| set2_3 | IGHV3- | IGHJ4 | 5 | 42 | 138 | 220 | 307 | 423 | 1197 |
| | 73 | | | | | | | | |
| set2_4 | IGHV3- | IGHJ3 | 5 | 42 | 138 | 220 | 307 | 424 | 1198 |
| | 73 | | | | | | | | |
| set2_5 | IGHV3- | IGHJ4 | 7 | 44 | 140 | 222 | 309 | 425 | 1197 |
| | 15 | | | | | | | | |
| set2_6 | IGHV3- | IGHJ4 | 2 | 45 | 141 | 223 | 310 | 426 | 1197 |
| | 30 | | | | | | | | |
| set2_7 | IGHV4- | IGHJ4 | 8 | 46 | 142 | 224 | 311 | 427 | 1199 |
| | 61 | | | | | | | | |
| set2_8 | IGHV4- | IGHJ4 | 9 | 47 | 143 | 225 | 312 | 428 | 1197 |
| | 59 | | | | | | | | |
| set2_9 | IGHV3- | IGHJ4 | 7 | 48 | 140 | 226 | 313 | 429 | 1197 |
| | 15 | | | | | | | | |
| set2_10 | IGHV4- | IGHJ4 | 10 | 47 | 144 | 225 | 314 | 430 | 1197 |
| | 59 | | | | | | | | |
| set2_11 | IGHV4- | IGHJ4 | 11 | 49 | 145 | 227 | 315 | 431 | 1197 |
| | 39 | | | | | | | | |
| set2_12 | IGHV3- | IGHJ4 | 12 | 50 | 146 | 228 | 304 | 432 | 1197 |
| | 23 | | | | | | | | |
| set2_13 | IGHV5- | IGHJ4 | 13 | 51 | 147 | 229 | 316 | 433 | 1197 |
| | 51 | | | | | | | | |
| set2_14 | IGHV3- | IGHJ6 | 7 | 44 | 140 | 222 | 309 | 434 | 1200 |
| | 15 | | | | | | | | |
| set2_15 | IGHV1- | IGHJ4 | 14 | 52 | 148 | 230 | 317 | 435 | 1197 |
| | 2 | | | | | | | | |
| set2_16 | IGHV4- | IGHJ5 | 15 | 53 | 149 | 231 | 318 | 436 | 1197 |
| | 34 | | | | | | | | |
| set2_17 | IGHV4- | IGHJ4 | 15 | 54 | 150 | 231 | 319 | 437 | 1201 |
| | 34 | | | | | | | | |
| set2_18 | IGHV4- | IGHJ4 | 9 | 47 | 143 | 225 | 320 | 438 | 1197 |
| | 59 | | | | | | | | |
| set2_19 | IGHV4- | IGHJ4 | 9 | 47 | 143 | 225 | 312 | 439 | 1197 |
| | 59 | | | | | | | | |
| set2_20 | IGHV4- | IGHJ4 | 16 | 55 | 151 | 232 | 321 | 440 | 1197 |
| | 4 | | | | | | | | |
| set2_21 | IGHV1- | IGHJ4 | 14 | 56 | 152 | 233 | 322 | 441 | 1202 |
| | 18 | | | | | | | | |
| set2_22 | IGHV1- | IGHJ5 | 14 | 57 | 153 | 234 | 323 | 442 | 1197 |
| | 8 | | | | | | | | |
| set2_23 | IGHV3- | IGHJ4 | 5 | 42 | 138 | 220 | 307 | 443 | 1197 |
| | 73 | | | | | | | | |
| set2_24 | IGHV4- | IGHJ5 | 16 | 55 | 151 | 232 | 321 | 444 | 1197 |
| | 4 | | | | | | | | |
| set2_25 | IGHV3- | IGHJ4 | 2 | 39 | 135 | 218 | 304 | 445 | 1197 |
| | 33 | | | | | | | | |
| set2_26 | IGHV3- | IGHJ4 | 6 | 58 | 154 | 235 | 324 | 446 | 1197 |
| | 48 | | | | | | | | |
| set2_27 | IGHV3- | IGHJ4 | 6 | 59 | 155 | 236 | 304 | 447 | 1197 |
| | 66 | | | | | | | | |
| set2_28 | IGHV3- | IGHJ5 | 6 | 60 | 156 | 237 | 325 | 448 | 1197 |
| | 74 | | | | | | | | |
| set2_29 | IGHV5- | IGHJ4 | 17 | 61 | 157 | 229 | 326 | 449 | 1197 |
| | 51 | | | | | | | | |
| set2_30 | IGHV3- | IGHJ6 | 2 | 39 | 135 | 218 | 304 | 450 | 1196 |
| | 33 | | | | | | | | |
| set2_31 | IGHV3- | IGHJ4 | 18 | 62 | 158 | 238 | 327 | 451 | 1197 |
| | 9 | | | | | | | | |
| set2_32 | IGHV5- | IGHJ3 | 17 | 63 | 157 | 239 | 328 | 452 | 1198 |
| | 51 | | | | | | | | |
| set2_33 | IGHV3- | IGHJ4 | 2 | 38 | 135 | 216 | 304 | 453 | 1197 |
| | 30 | | | | | | | | |
| set2_34 | IGHV3- | IGHJ4 | 19 | 64 | 159 | 240 | 329 | 454 | 1197 |
| | 74 | | | | | | | | |
| set2_35 | IGHV3- | IGHJ4 | 20 | 65 | 160 | 241 | 330 | 455 | 1197 |
| | 11 | | | | | | | | |
| set2_36 | IGHV3- | IGHJ4 | 6 | 59 | 155 | 236 | 304 | 456 | 1197 |
| | 66 | | | | | | | | |
| set2_37 | IGHV5- | IGHJ3 | 17 | 61 | 157 | 229 | 326 | 457 | 1198 |
| | 51 | | | | | | | | |
| set2_38 | IGHV3- | IGHJ4 | 2 | 39 | 135 | 218 | 304 | 458 | 1197 |
| | 33 | | | | | | | | |
| set2_39 | IGHV3- | IGHJ4 | 2 | 38 | 135 | 216 | 304 | 459 | 1197 |
| | 30 | | | | | | | | |
| set2_40 | IGHV3- | IGHJ4 | 2 | 38 | 135 | 216 | 304 | 460 | 1197 |
| | 30 | | | | | | | | |
| set2_41 | IGHV3- | IGHJ4 | 2 | 38 | 135 | 216 | 304 | 461 | 1197 |
| | 30 | | | | | | | | |
| set2_42 | IGHV3- | IGHJ5 | 2 | 38 | 135 | 216 | 304 | 462 | 1197 |
| | 30 | | | | | | | | |
| set2_43 | IGHV3- | IGHJ2 | 2 | 39 | 135 | 218 | 304 | 463 | 1203 |
| | 33 | | | | | | | | |
| set2_44 | IGHV3- | IGHJ4 | 2 | 38 | 135 | 216 | 304 | 464 | 1197 |
| | 30 | | | | | | | | |
| set2_45 | IGHV3- | IGHJ4 | 2 | 39 | 135 | 218 | 304 | 465 | 1197 |
| | 33 | | | | | | | | |
| set2_46 | IGHV3- | IGHJ6 | 2 | 38 | 135 | 216 | 304 | 466 | 1196 |
| | 30-3 | | | | | | | | |
| set2_47 | IGHV3- | IGHJ3 | 2 | 38 | 135 | 216 | 304 | 467 | 1198 |
| | 30-3 | | | | | | | | |
| set2_48 | IGHV3- | IGHJ4 | 21 | 59 | 155 | 236 | 304 | 468 | 1197 |
| | 66 | | | | | | | | |
| set2_49 | IGHV3- | IGHJ4 | 2 | 39 | 135 | 218 | 304 | 469 | 1197 |
| | 33 | | | | | | | | |
| set2_50 | IGHV3- | IGHJ4 | 2 | 38 | 135 | 216 | 304 | 470 | 1197 |
| | 30 | | | | | | | | |
| set2_51 | IGHV4- | IGHJ4 | 15 | 66 | 161 | 242 | 312 | 471 | 1197 |
| | 34 | | | | | | | | |
| set2_52 | IGHV3- | IGHJ3 | 2 | 38 | 135 | 216 | 304 | 472 | 1198 |
| | 30 | | | | | | | | |
| set2_53 | IGHV3- | IGHJ5 | 2 | 38 | 135 | 216 | 304 | 473 | 1197 |
| | 30 | | | | | | | | |
| set2_54 | IGHV4- | IGHJ4 | 22 | 67 | 143 | 232 | 331 | 474 | 1197 |
| | 30-2 | | | | | | | | |
| set2_55 | IGHV3- | IGHJ4 | 2 | 38 | 135 | 216 | 304 | 475 | 1197 |
| | 30 | | | | | | | | |
| set2_56 | IGHV3- | IGHJ4 | 2 | 39 | 135 | 218 | 304 | 476 | 1197 |
| | 33 | | | | | | | | |
| set2_57 | IGHV3- | IGHJ4 | 2 | 39 | 135 | 218 | 304 | 477 | 1197 |
| | 33 | | | | | | | | |
| set2_58 | IGHV3- | IGHJ6 | 2 | 68 | 162 | 243 | 332 | 478 | 1204 |
| | 30 | | | | | | | | |
| set2_59 | IGHV3- | IGHJ6 | 2 | 39 | 135 | 216 | 304 | 479 | 1196 |
| | 30 | | | | | | | | |
| set2_60 | IGHV3- | IGHJ4 | 21 | 59 | 155 | 236 | 304 | 480 | 1197 |
| | 53 | | | | | | | | |
| set2_61 | IGHV3- | IGHJ2 | 2 | 38 | 135 | 216 | 304 | 481 | 1203 |
| | 30 | | | | | | | | |
| set2_62 | IGHV3- | IGHJ4 | 2 | 39 | 135 | 216 | 304 | 482 | 1197 |
| | 30 | | | | | | | | |
| set2_63 | IGHV1- | IGHJ3 | 23 | 69 | 163 | 244 | 333 | 483 | 1205 |
| | 18 | | | | | | | | |
| set2_64 | IGHV3- | IGHJ4 | 6 | 58 | 154 | 235 | 324 | 484 | 1197 |
| | 48 | | | | | | | | |
| set2_65 | IGHV1- | IGHJ4 | 14 | 70 | 164 | 245 | 334 | 485 | 1197 |
| | 18 | | | | | | | | |
| set2_66 | IGHV3- | IGHJ4 | 2 | 38 | 135 | 216 | 304 | 486 | 1197 |
| | 30 | | | | | | | | |
| set2_67 | IGHV3- | IGHJ5 | 2 | 38 | 135 | 216 | 304 | 487 | 1197 |
| | 30 | | | | | | | | |
| set2_68 | IGHV3- | IGHJ4 | 2 | 38 | 135 | 216 | 304 | 488 | 1197 |
| | 30 | | | | | | | | |
| set2_69 | IGHV1- | IGHJ4 | 4 | 71 | 165 | 246 | 335 | 489 | 1197 |
| | 69 | | | | | | | | |
| set2_70 | IGHV3- | IGHJ4 | 2 | 39 | 135 | 216 | 304 | 490 | 1197 |
| | 30 | | | | | | | | |
| set2_71 | IGHV3- | IGHJ4 | 2 | 38 | 135 | 216 | 304 | 491 | 1197 |
| | 30 | | | | | | | | |
| set2_72 | IGHV3- | IGHJ4 | 2 | 38 | 135 | 216 | 304 | 492 | 1197 |
| | 30 | | | | | | | | |
| set2_73 | IGHV3- | IGHJ3 | 2 | 38 | 135 | 216 | 304 | 493 | 1198 |
| | 30-3 | | | | | | | | |
| set2_74 | IGHV3- | IGHJ4 | 2 | 38 | 135 | 216 | 304 | 494 | 1197 |
| | 30 | | | | | | | | |
| set2_75 | IGHV3- | IGHJ4 | 6 | 58 | 154 | 235 | 324 | 495 | 1197 |
| | 48 | | | | | | | | |
| set2_76 | IGHV3- | IGHJ4 | 2 | 39 | 135 | 216 | 304 | 496 | 1197 |
| | 30 | | | | | | | | |
| set2_77 | IGHV3- | IGHJ4 | 2 | 39 | 135 | 216 | 304 | 497 | 1197 |
| | 30 | | | | | | | | |
| set2_78 | IGHV3- | IGHJ4 | 2 | 39 | 135 | 216 | 304 | 498 | 1197 |
| | 30 | | | | | | | | |
| set2_79 | IGHV3- | IGHJ4 | 2 | 38 | 135 | 216 | 304 | 499 | 1197 |
| | 30-3 | | | | | | | | |
| set2_80 | IGHV3- | IGHJ6 | 2 | 38 | 135 | 216 | 304 | 500 | 1196 |
| | 30 | | | | | | | | |
| set2_81 | IGHV3- | IGHJ6 | 2 | 39 | 135 | 218 | 304 | 501 | 1206 |
| | 33 | | | | | | | | |
| set2_82 | IGHV3- | IGHJ4 | 2 | 38 | 135 | 216 | 304 | 502 | 1197 |
| | 30-3 | | | | | | | | |
| set2_83 | IGHV3- | IGHJ3 | 2 | 38 | 135 | 216 | 304 | 503 | 1198 |
| | 30 | | | | | | | | |
| set2_84 | IGHV2- | IGHJ4 | 24 | 72 | 166 | 247 | 336 | 504 | 1197 |
| | 5 | | | | | | | | |
| set2_85 | IGHV3- | IGHJ4 | 2 | 73 | 135 | 248 | 337 | 505 | 1207 |
| | 30 | | | | | | | | |
| set2_86 | IGHV3- | IGHJ5 | 6 | 59 | 155 | 236 | 338 | 506 | 1197 |
| | 53 | | | | | | | | |
| set2_87 | IGHV3- | IGHJ6 | 2 | 38 | 135 | 216 | 304 | 507 | 1196 |
| | 30-3 | | | | | | | | |
| set2_88 | IGHV2- | IGHJ4 | 25 | 74 | 167 | 249 | 339 | 508 | 1197 |
| | 70 | | | | | | | | |
| set2_89 | IGHV2- | IGHJ4 | 24 | 72 | 166 | 247 | 336 | 509 | 1197 |
| | 5 | | | | | | | | |
| set2_90 | IGHV3- | IGHJ6 | 2 | 39 | 135 | 218 | 304 | 510 | 1196 |
| | 33 | | | | | | | | |
| set2_91 | IGHV3- | IGHJ4 | 2 | 75 | 168 | 250 | 340 | 511 | 1197 |
| | 33 | | | | | | | | |
| set2_92 | IGHV3- | IGHJ4 | 7 | 58 | 169 | 251 | 341 | 512 | 1197 |
| | 21 | | | | | | | | |
| set2_93 | IGHV3- | IGHJ6 | 2 | 39 | 135 | 216 | 304 | 513 | 1196 |
| | 30 | | | | | | | | |
| set2_94 | IGHV1- | IGHJ4 | 14 | 76 | 170 | 252 | 342 | 514 | 1197 |
| | 46 | | | | | | | | |
| set2_95 | IGHV4- | IGHJ6 | 15 | 66 | 161 | 242 | 312 | 515 | 1196 |
| | 34 | | | | | | | | |
| set2_96 | IGHV2- | IGHJ4 | 25 | 74 | 167 | 249 | 339 | 516 | 1197 |
| | 70 | | | | | | | | |
| set2_97 | IGHV3- | IGHJ4 | 6 | 43 | 171 | 253 | 343 | 517 | 1197 |
| | 7 | | | | | | | | |
| set2_98 | IGHV1- | IGHJ5 | 14 | 70 | 164 | 245 | 334 | 518 | 1197 |
| | 18 | | | | | | | | |
| set2_99 | IGHV3- | IGHJ4 | 26 | 77 | 172 | 216 | 344 | 519 | 1197 |
| | 30-3 | | | | | | | | |
| set2_100 | IGHV3- | IGHJ6 | 21 | 59 | 173 | 236 | 304 | 520 | 1208 |
| | 53 | | | | | | | | |
| set2_101 | IGHV4- | IGHJ6 | 11 | 78 | 174 | 225 | 345 | 521 | 1196 |
| | 39 | | | | | | | | |
| set2_102 | IGHV3- | IGHJ3 | 21 | 59 | 155 | 236 | 304 | 522 | 1198 |
| | 53 | | | | | | | | |
| set2_103 | IGHV4- | IGHJ6 | 15 | 66 | 161 | 242 | 312 | 523 | 1196 |
| | 34 | | | | | | | | |
| set2_104 | IGHV3- | IGHJ4 | 12 | 38 | 146 | 254 | 304 | 524 | 1197 |
| | 23 | | | | | | | | |
| set2_105 | IGHV3- | IGHJ4 | 2 | 38 | 135 | 216 | 304 | 525 | 1197 |
| | 30 | | | | | | | | |
| set2_106 | IGHV1- | IGHJ3 | 14 | 70 | 164 | 245 | 334 | 526 | 1198 |
| | 18 | | | | | | | | |
| set2_107 | IGHV4- | IGHJ6 | 16 | 79 | 151 | 255 | 346 | 527 | 1209 |
| | 4 | | | | | | | | |
| set2_108 | IGHV4- | IGHJ4 | 15 | 66 | 161 | 242 | 312 | 528 | 1197 |
| | 34 | | | | | | | | |
| set2_109 | IGHV4- | IGHJ4 | 9 | 47 | 175 | 256 | 347 | 529 | 1197 |
| | 4 | | | | | | | | |
| set2_110 | IGHV3- | IGHJ5 | 6 | 80 | 171 | 253 | 348 | 530 | 1197 |
| | 7 | | | | | | | | |
| set2_111 | IGHV2- | IGHJ4 | 25 | 74 | 167 | 249 | 339 | 531 | 1197 |
| | 70 | | | | | | | | |
| set2_112 | IGHV2- | IGHJ5 | 24 | 72 | 166 | 247 | 336 | 532 | 1197 |
| | 5 | | | | | | | | |
| set2_113 | IGHV2- | IGHJ5 | 27 | 81 | 166 | 247 | 349 | 533 | 1197 |
| | 5 | | | | | | | | |
| set2_114 | IGHV3- | IGHJ6 | 2 | 39 | 135 | 218 | 304 | 534 | 1196 |
| | 33 | | | | | | | | |
| set2_115 | IGHV5- | IGHJ6 | 17 | 61 | 157 | 229 | 326 | 535 | 1196 |
| | 51 | | | | | | | | |
| set2_116 | IGHV3- | IGHJ4 | 2 | 38 | 135 | 216 | 304 | 536 | 1197 |
| | 30-3 | | | | | | | | |
| set2_117 | IGHV3- | IGHJ4 | 2 | 82 | 176 | 257 | 350 | 537 | 1202 |
| | 30 | | | | | | | | |
| set2_118 | IGHV3- | IGHJ4 | 2 | 38 | 135 | 216 | 304 | 538 | 1197 |
| | 30 | | | | | | | | |
| set2_119 | IGHV3- | IGHJ3 | 2 | 38 | 135 | 216 | 304 | 539 | 1198 |
| | 30 | | | | | | | | |
| set2_120 | IGHV3- | IGHJ4 | 2 | 83 | 135 | 216 | 304 | 540 | 1197 |
| | 30 | | | | | | | | |
| set2_121 | IGHV3- | IGHJ6 | 2 | 39 | 135 | 218 | 304 | 541 | 1196 |
| | 33 | | | | | | | | |
| set2_122 | IGHV1- | IGHJ4 | 14 | 84 | 148 | 230 | 351 | 542 | 1197 |
| | 2 | | | | | | | | |
| set2_123 | IGHV5- | IGHJ4 | 17 | 61 | 157 | 229 | 326 | 543 | 1197 |
| | 51 | | | | | | | | |
| set2_124 | IGHV3- | IGHJ4 | 7 | 85 | 169 | 251 | 341 | 544 | 1197 |
| | 21 | | | | | | | | |
| set2_125 | IGHV3- | IGHJ4 | 2 | 38 | 135 | 216 | 304 | 545 | 1197 |
| | 30 | | | | | | | | |
| set2_126 | IGHV2- | IGHJ4 | 24 | 72 | 166 | 247 | 336 | 546 | 1197 |
| | 5 | | | | | | | | |
| set2_127 | IGHV4- | IGHJ4 | 9 | 47 | 143 | 225 | 312 | 547 | 1197 |
| | 59 | | | | | | | | |
| set2_128 | IGHV3- | IGHJ4 | 2 | 86 | 177 | 216 | 352 | 548 | 1197 |
| | 30-3 | | | | | | | | |
| set2_129 | IGHV3- | IGHJ3 | 2 | 38 | 135 | 216 | 304 | 549 | 1198 |
| | 30 | | | | | | | | |
| set2_130 | IGHV2- | IGHJ3 | 24 | 72 | 166 | 247 | 336 | 550 | 1198 |
| | 5 | | | | | | | | |
| set2_131 | IGHV3- | IGHJ4 | 2 | 87 | 135 | 258 | 353 | 551 | 1197 |
| | 33 | | | | | | | | |
| set2_132 | IGHV3- | IGHJ4 | 2 | 39 | 135 | 216 | 304 | 552 | 1197 |
| | 30 | | | | | | | | |
| set2_133 | IGHV3- | IGHJ4 | 2 | 39 | 135 | 216 | 304 | 553 | 1197 |
| | 30 | | | | | | | | |
| set2_134 | IGHV3- | IGHJ3 | 2 | 39 | 135 | 218 | 304 | 554 | 1198 |
| | 33 | | | | | | | | |
| set2_135 | IGHV3- | IGHJ4 | 2 | 39 | 135 | 218 | 304 | 555 | 1197 |
| | 33 | | | | | | | | |
| set2_136 | IGHV3- | IGHJ3 | 2 | 38 | 135 | 216 | 304 | 556 | 1198 |
| | 30 | | | | | | | | |
| set2_137 | IGHV2- | IGHJ4 | 25 | 74 | 167 | 249 | 339 | 557 | 1197 |
| | 70 | | | | | | | | |
| set2_138 | IGHV3- | IGHJ4 | 2 | 39 | 135 | 218 | 354 | 558 | 1197 |
| | 33 | | | | | | | | |
| set2_139 | IGHV3- | IGHJ6 | 2 | 39 | 135 | 259 | 304 | 559 | 1196 |
| | 30 | | | | | | | | |
| set2_140 | IGHV4- | IGHJ4 | 11 | 88 | 145 | 225 | 355 | 560 | 1197 |
| | 39 | | | | | | | | |
| set2_141 | IGHV4- | IGHJ4 | 15 | 66 | 161 | 242 | 312 | 561 | 1197 |
| | 34 | | | | | | | | |
| set2_142 | IGHV3- | IGHJ6 | 2 | 39 | 135 | 218 | 304 | 562 | 1196 |
| | 33 | | | | | | | | |
| set2_143 | IGHV3- | IGHJ4 | 2 | 38 | 135 | 216 | 304 | 563 | 1197 |
| | 30 | | | | | | | | |
| set2_144 | IGHV3- | IGHJ4 | 2 | 38 | 135 | 216 | 304 | 564 | 1197 |
| | 30-3 | | | | | | | | |
| set2_145 | IGHV3- | IGHJ4 | 2 | 39 | 135 | 218 | 304 | 565 | 1197 |
| | 33 | | | | | | | | |
| set2_146 | IGHV3- | IGHJ6 | 2 | 38 | 135 | 216 | 304 | 566 | 1196 |
| | 30-3 | | | | | | | | |
| set2_147 | IGHV2- | IGHJ4 | 24 | 72 | 166 | 247 | 336 | 567 | 1197 |
| | 5 | | | | | | | | |
| set2_148 | IGHV3- | IGHJ4 | 2 | 38 | 135 | 216 | 304 | 568 | 1197 |
| | 30-3 | | | | | | | | |
| set2_149 | IGHV3- | IGHJ6 | 2 | 39 | 135 | 218 | 304 | 569 | 1196 |
| | 33 | | | | | | | | |
| set2_150 | IGHV4- | IGHJ4 | 9 | 47 | 143 | 225 | 312 | 570 | 1197 |
| | 59 | | | | | | | | |
| set2_151 | IGHV3- | IGHJ6 | 2 | 38 | 135 | 216 | 304 | 571 | 1200 |
| | 30 | | | | | | | | |
| set2_152 | IGHV3- | IGHJ6 | 2 | 38 | 135 | 216 | 304 | 572 | 1196 |
| | 30-3 | | | | | | | | |
| set2_153 | IGHV3- | IGHJ4 | 2 | 39 | 135 | 216 | 304 | 573 | 1197 |
| | 30 | | | | | | | | |
| set2_154 | IGHV3- | IGHJ4 | 28 | 89 | 178 | 260 | 356 | 574 | 1197 |
| | 49 | | | | | | | | |
| set2_155 | IGHV3- | IGHJ6 | 2 | 39 | 135 | 218 | 304 | 575 | 1196 |
| | 33 | | | | | | | | |
| set2_156 | IGHV3- | IGHJ6 | 2 | 38 | 135 | 216 | 304 | 576 | 1196 |
| | 30 | | | | | | | | |
| set2_157 | IGHV3- | IGHJ4 | 2 | 39 | 135 | 218 | 304 | 577 | 1197 |
| | 33 | | | | | | | | |
| set2_158 | IGHV3- | IGHJ4 | 12 | 38 | 146 | 254 | 304 | 578 | 1197 |
| | 23 | | | | | | | | |
| set2_159 | IGHV3- | IGHJ4 | 6 | 43 | 171 | 253 | 343 | 579 | 1197 |
| | 7 | | | | | | | | |
| set2_160 | IGHV1- | IGHJ4 | 4 | 90 | 179 | 261 | 357 | 580 | 1197 |
| | 69 | | | | | | | | |
| set2_161 | IGHV3- | IGHJ3 | 2 | 91 | 135 | 216 | 304 | 581 | 1198 |
| | 30 | | | | | | | | |
| set2_162 | IGHV4- | IGHJ4 | 15 | 66 | 161 | 242 | 312 | 582 | 1197 |
| | 34 | | | | | | | | |
| set2_163 | IGHV2- | IGHJ4 | 25 | 74 | 167 | 249 | 339 | 583 | 1197 |
| | 70 | | | | | | | | |
| set2_164 | IGHV3- | IGHJ4 | 2 | 39 | 135 | 218 | 304 | 584 | 1197 |
| | 33 | | | | | | | | |
| set2_165 | IGHV4- | IGHJ6 | 15 | 66 | 161 | 242 | 312 | 585 | 1196 |
| | 34 | | | | | | | | |
| set2_166 | IGHV3- | IGHJ4 | 2 | 39 | 135 | 216 | 304 | 586 | 1197 |
| | 30 | | | | | | | | |
| set2_167 | IGHV3- | IGHJ3 | 2 | 39 | 135 | 218 | 304 | 587 | 1198 |
| | 33 | | | | | | | | |
| set2_168 | IGHV3- | IGHJ4 | 2 | 39 | 135 | 218 | 304 | 588 | 1197 |
| | 33 | | | | | | | | |
| set2_169 | IGHV1- | IGHJ4 | 14 | 84 | 148 | 230 | 351 | 589 | 1197 |
| | 2 | | | | | | | | |
| set2_170 | IGHV3- | IGHJ6 | 2 | 39 | 135 | 218 | 304 | 590 | 1196 |
| | 33 | | | | | | | | |
| set2_171 | IGHV3- | IGHJ4 | 2 | 39 | 135 | 216 | 304 | 591 | 1197 |
| | 30 | | | | | | | | |
| set2_172 | IGHV3- | IGHJ6 | 2 | 38 | 135 | 216 | 304 | 592 | 1196 |
| | 30 | | | | | | | | |
| set2_173 | IGHV3- | IGHJ3 | 2 | 39 | 135 | 218 | 304 | 593 | 1198 |
| | 33 | | | | | | | | |
| set2_174 | IGHV3- | IGHJ4 | 18 | 92 | 180 | 262 | 358 | 594 | 1210 |
| | 9 | | | | | | | | |
| set2_175 | IGHV3- | IGHJ4 | 2 | 39 | 135 | 218 | 304 | 595 | 1197 |
| | 33 | | | | | | | | |
| set2_176 | IGHV3- | IGHJ4 | 2 | 38 | 135 | 216 | 304 | 596 | 1197 |
| | 30 | | | | | | | | |
| set2_177 | IGHV3- | IGHJ6 | 2 | 39 | 135 | 218 | 304 | 597 | 1196 |
| | 33 | | | | | | | | |
| set2_178 | IGHV3- | IGHJ3 | 7 | 58 | 169 | 251 | 341 | 598 | 1198 |
| | 21 | | | | | | | | |
| set2_179 | IGHV3- | IGHJ3 | 2 | 93 | 181 | 263 | 359 | 599 | 1211 |
| | 30-3 | | | | | | | | |
| set2_180 | IGHV2- | IGHJ3 | 25 | 74 | 167 | 249 | 339 | 600 | 1198 |
| | 70 | | | | | | | | |
| set2_181 | IGHV3- | IGHJ4 | 2 | 39 | 135 | 216 | 304 | 601 | 1197 |
| | 30 | | | | | | | | |
| set2_182 | IGHV3- | IGHJ4 | 2 | 38 | 135 | 216 | 304 | 602 | 1197 |
| | 30 | | | | | | | | |
| set2_183 | IGHV3- | IGHJ4 | 2 | 38 | 135 | 216 | 304 | 603 | 1197 |
| | 30-3 | | | | | | | | |
| set2_184 | IGHV3- | IGHJ4 | 2 | 39 | 135 | 218 | 304 | 604 | 1197 |
| | 33 | | | | | | | | |
| set2_185 | IGHV1- | IGHJ4 | 14 | 70 | 164 | 245 | 334 | 605 | 1197 |
| | 18 | | | | | | | | |
| set2_186 | IGHV3- | IGHJ3 | 2 | 38 | 135 | 216 | 304 | 606 | 1198 |
| | 30-3 | | | | | | | | |
| set2_187 | IGHV4- | IGHJ4 | 9 | 47 | 143 | 225 | 312 | 607 | 1197 |
| | 59 | | | | | | | | |
| set2_188 | IGHV2- | IGHJ2 | 29 | 94 | 182 | 264 | 360 | 608 | 1203 |
| | 26 | | | | | | | | |
| set2_189 | IGHV3- | IGHJ4 | 18 | 92 | 158 | 238 | 361 | 609 | 1197 |
| | 9 | | | | | | | | |
| set2_190 | IGHV3- | IGHJ6 | 2 | 39 | 135 | 216 | 304 | 610 | 1196 |
| | 30 | | | | | | | | |
| set2_191 | IGHV5- | IGHJ6 | 17 | 61 | 157 | 229 | 326 | 611 | 1196 |
| | 51 | | | | | | | | |
| set2_192 | IGHV3- | IGHJ4 | 2 | 39 | 135 | 216 | 304 | 612 | 1197 |
| | 30 | | | | | | | | |
| set2_193 | IGHV3- | IGHJ4 | 12 | 38 | 146 | 254 | 304 | 613 | 1197 |
| | 23 | | | | | | | | |
| set2_194 | IGHV1- | IGHJ4 | 14 | 70 | 164 | 245 | 334 | 614 | 1197 |
| | 18 | | | | | | | | |
| set2_195 | IGHV1- | IGHJ6 | 14 | 70 | 164 | 245 | 334 | 615 | 1196 |
| | 18 | | | | | | | | |
| set2_196 | IGHV3- | IGHJ6 | 2 | 39 | 135 | 218 | 304 | 616 | 1196 |
| | 33 | | | | | | | | |
| set2_197 | IGHV3- | IGHJ4 | 2 | 95 | 135 | 265 | 304 | 617 | 1197 |
| | 30-3 | | | | | | | | |
| set2_198 | IGHV3- | IGHJ6 | 2 | 39 | 135 | 216 | 304 | 618 | 1196 |
| | 30 | | | | | | | | |
| set2_199 | IGHV2- | IGHJ4 | 25 | 74 | 167 | 249 | 339 | 619 | 1197 |
| | 70 | | | | | | | | |
| set2_200 | IGHV3- | IGHJ6 | 2 | 39 | 135 | 218 | 304 | 620 | 1196 |
| | 33 | | | | | | | | |
| set2_201 | IGHV3- | IGHJ4 | 2 | 38 | 135 | 216 | 304 | 621 | 1197 |
| | 30 | | | | | | | | |
| set2_202 | IGHV3- | IGHJ4 | 2 | 39 | 135 | 218 | 304 | 622 | 1197 |
| | 33 | | | | | | | | |
| set2_203 | IGHV3- | IGHJ6 | 2 | 39 | 135 | 216 | 304 | 623 | 1196 |
| | 30 | | | | | | | | |
| set2_204 | IGHV3- | IGHJ6 | 12 | 96 | 183 | 266 | 362 | 569 | 1196 |
| | 23 | | | | | | | | |
| set2_205 | IGHV3- | IGHJ6 | 2 | 39 | 135 | 218 | 304 | 624 | 1196 |
| | 33 | | | | | | | | |
| set2_206 | IGHV3- | IGHJ6 | 2 | 39 | 135 | 216 | 304 | 625 | 1196 |
| | 30 | | | | | | | | |
| set2_207 | IGHV3- | IGHJ3 | 2 | 39 | 135 | 218 | 304 | 626 | 1198 |
| | 33 | | | | | | | | |
| set2_208 | IGHV3- | IGHJ4 | 2 | 97 | 135 | 216 | 304 | 627 | 1197 |
| | 30-3 | | | | | | | | |
| set2_209 | IGHV5- | IGHJ4 | 17 | 61 | 157 | 229 | 326 | 628 | 1197 |
| | 51 | | | | | | | | |
| set2_210 | IGHV2- | IGHJ4 | 25 | 74 | 167 | 249 | 339 | 629 | 1197 |
| | 70 | | | | | | | | |
| set2_211 | IGHV3- | IGHJ4 | 2 | 38 | 135 | 216 | 304 | 630 | 1197 |
| | 30 | | | | | | | | |
| set2_212 | IGHV3- | IGHJ4 | 2 | 39 | 135 | 218 | 304 | 631 | 1197 |
| | 33 | | | | | | | | |
| set2_213 | IGHV3- | IGHJ4 | 2 | 39 | 135 | 218 | 304 | 632 | 1197 |
| | 33 | | | | | | | | |
| set2_214 | IGHV3- | IGHJ6 | 2 | 39 | 135 | 216 | 304 | 633 | 1196 |
| | 30 | | | | | | | | |
| set2_215 | IGHV3- | IGHJ4 | 2 | 39 | 135 | 218 | 304 | 634 | 1197 |
| | 33 | | | | | | | | |
| set2_216 | IGHV3- | IGHJ4 | 2 | 39 | 135 | 218 | 304 | 635 | 1197 |
| | 33 | | | | | | | | |
| set2_217 | IGHV3- | IGHJ4 | 2 | 39 | 135 | 218 | 304 | 636 | 1197 |
| | 33 | | | | | | | | |
| set2_218 | IGHV5- | IGHJ4 | 17 | 61 | 157 | 229 | 326 | 637 | 1197 |
| | 51 | | | | | | | | |
| set2_219 | IGHV2- | IGHJ6 | 24 | 72 | 166 | 247 | 363 | 638 | 1196 |
| | 5 | | | | | | | | |
| set2_220 | IGHV1- | IGHJ5 | 30 | 98 | 184 | 267 | 364 | 639 | 1197 |
| | 24 | | | | | | | | |
| set2_221 | IGHV3- | IGHJ6 | 2 | 39 | 135 | 218 | 304 | 640 | 1200 |
| | 33 | | | | | | | | |
| set2_222 | IGHV4- | IGHJ6 | 15 | 66 | 161 | 242 | 312 | 641 | 1200 |
| | 34 | | | | | | | | |
| set2_223 | IGHV3- | IGHJ4 | 2 | 38 | 135 | 216 | 304 | 642 | 1197 |
| | 30 | | | | | | | | |
| set2_224 | IGHV3- | IGHJ6 | 2 | 39 | 135 | 218 | 304 | 643 | 1196 |
| | 33 | | | | | | | | |
| set2_225 | IGHV3- | IGHJ6 | 2 | 99 | 135 | 268 | 365 | 644 | 1212 |
| | 33 | | | | | | | | |
| set2_226 | IGHV3- | IGHJ4 | 6 | 43 | 171 | 253 | 343 | 645 | 1197 |
| | 7 | | | | | | | | |
| set2_227 | IGHV4- | IGHJ4 | 9 | 47 | 143 | 269 | 366 | 646 | 1213 |
| | 59 | | | | | | | | |
| set2_228 | IGHV3- | IGHJ4 | 18 | 92 | 158 | 238 | 361 | 647 | 1197 |
| | 9 | | | | | | | | |
| set2_229 | IGHV3- | IGHJ4 | 2 | 38 | 135 | 216 | 304 | 648 | 1197 |
| | 30 | | | | | | | | |
| set2_230 | IGHV2- | IGHJ6 | 24 | 72 | 166 | 247 | 336 | 649 | 1196 |
| | 5 | | | | | | | | |
| set2_231 | IGHV3- | IGHJ4 | 2 | 39 | 135 | 218 | 304 | 650 | 1197 |
| | 33 | | | | | | | | |
| set2_232 | IGHV3- | IGHJ6 | 2 | 39 | 135 | 218 | 304 | 651 | 1200 |
| | 33 | | | | | | | | |
| set2_233 | IGHV3- | IGHJ4 | 2 | 38 | 135 | 216 | 304 | 652 | 1197 |
| | 30 | | | | | | | | |
| set2_234 | IGHV3- | IGHJ3 | 2 | 38 | 135 | 216 | 304 | 653 | 1198 |
| | 30 | | | | | | | | |
| set2_235 | IGHV3- | IGHJ6 | 2 | 38 | 135 | 216 | 304 | 654 | 1196 |
| | 30 | | | | | | | | |
| set2_236 | IGHV1- | IGHJ3 | 31 | 100 | 185 | 270 | 367 | 655 | 1198 |
| | 58 | | | | | | | | |
| set2_237 | IGHV3- | IGHJ4 | 2 | 38 | 135 | 216 | 304 | 656 | 1197 |
| | 30 | | | | | | | | |
| set2_238 | IGHV3- | IGHJ4 | 7 | 58 | 169 | 251 | 368 | 657 | 1197 |
| | 21 | | | | | | | | |
| set2_239 | IGHV3- | IGHJ4 | 6 | 43 | 171 | 253 | 343 | 658 | 1213 |
| | 7 | | | | | | | | |
| set2_240 | IGHV3- | IGHJ6 | 2 | 38 | 135 | 216 | 304 | 659 | 1196 |
| | 30 | | | | | | | | |
| set2_241 | IGHV3- | IGHJ4 | 2 | 38 | 135 | 216 | 304 | 660 | 1197 |
| | 30 | | | | | | | | |
| set2_242 | IGHV3- | IGHJ6 | 2 | 38 | 135 | 216 | 304 | 661 | 1196 |
| | 30 | | | | | | | | |
| set2_243 | IGHV3- | IGHJ4 | 2 | 39 | 135 | 218 | 304 | 662 | 1197 |
| | 33 | | | | | | | | |
| set2_244 | IGHV3- | IGHJ6 | 2 | 38 | 135 | 216 | 304 | 663 | 1200 |
| | 30 | | | | | | | | |
| set2_245 | IGHV3- | IGHJ6 | 2 | 38 | 135 | 216 | 304 | 664 | 1196 |
| | 30 | | | | | | | | |
| set2_246 | IGHV3- | IGHJ4 | 12 | 38 | 146 | 254 | 304 | 665 | 1197 |
| | 23 | | | | | | | | |
| set2_247 | IGHV1- | IGHJ4 | 14 | 70 | 164 | 245 | 334 | 666 | 1197 |
| | 18 | | | | | | | | |
| set2_248 | IGHV3- | IGHJ4 | 2 | 38 | 135 | 216 | 304 | 667 | 1197 |
| | 30 | | | | | | | | |
| set2_249 | IGHV3- | IGHJ3 | 2 | 101 | 135 | 216 | 369 | 668 | 1198 |
| | 30 | | | | | | | | |
| set2_250 | IGHV3- | IGHJ6 | 2 | 102 | 135 | 271 | 370 | 669 | 1196 |
| | 33 | | | | | | | | |
| set2_251 | IGHV3- | IGHJ4 | 6 | 43 | 171 | 253 | 343 | 670 | 1197 |
| | 7 | | | | | | | | |
| set2_252 | IGHV3- | IGHJ5 | 2 | 38 | 135 | 216 | 304 | 671 | 1197 |
| | 30-3 | | | | | | | | |
| set2_253 | IGHV3- | IGHJ4 | 2 | 39 | 135 | 216 | 304 | 672 | 1197 |
| | 30 | | | | | | | | |
| set2_254 | IGHV3- | IGHJ4 | 2 | 39 | 135 | 218 | 304 | 673 | 1197 |
| | 33 | | | | | | | | |
| set2_255 | IGHV3- | IGHJ4 | 6 | 43 | 139 | 221 | 308 | 674 | 1197 |
| | 74 | | | | | | | | |
| set2_256 | IGHV3- | IGHJ4 | 2 | 39 | 135 | 218 | 304 | 675 | 1197 |
| | 33 | | | | | | | | |
| set2_257 | IGHV3- | IGHJ4 | 2 | 39 | 135 | 218 | 304 | 676 | 1197 |
| | 33 | | | | | | | | |
| set2_258 | IGHV3- | IGHJ6 | 20 | 65 | 186 | 272 | 341 | 677 | 1200 |
| | 11 | | | | | | | | |
| set2_259 | IGHV3- | IGHJ4 | 2 | 39 | 135 | 218 | 304 | 678 | 1197 |
| | 33 | | | | | | | | |
| set2_260 | IGHV3- | IGHJ5 | 2 | 39 | 135 | 218 | 304 | 679 | 1197 |
| | 33 | | | | | | | | |
| set2_261 | IGHV3- | IGHJ4 | 2 | 39 | 135 | 218 | 304 | 680 | 1197 |
| | 33 | | | | | | | | |
| set2_262 | IGHV3- | IGHJ5 | 2 | 38 | 135 | 216 | 304 | 681 | 1197 |
| | 30 | | | | | | | | |
| set2_263 | IGHV3- | IGHJ6 | 2 | 38 | 135 | 216 | 304 | 682 | 1196 |
| | 30-3 | | | | | | | | |
| set2_264 | IGHV2- | IGHJ4 | 25 | 74 | 167 | 249 | 339 | 683 | 1197 |
| | 70 | | | | | | | | |
| set2_265 | IGHV3- | IGHJ4 | 6 | 43 | 139 | 221 | 308 | 684 | 1197 |
| | 74 | | | | | | | | |
| set2_266 | IGHV2- | IGHJ4 | 24 | 72 | 166 | 247 | 336 | 685 | 1197 |
| | 5 | | | | | | | | |
| set2_267 | IGHV3- | IGHJ4 | 2 | 38 | 135 | 216 | 304 | 686 | 1197 |
| | 30 | | | | | | | | |
| set2_268 | IGHV3- | IGHJ4 | 2 | 39 | 135 | 216 | 304 | 687 | 1197 |
| | 30 | | | | | | | | |
| set2_269 | IGHV3- | IGHJ4 | 2 | 39 | 135 | 216 | 304 | 688 | 1197 |
| | 30 | | | | | | | | |
| set2_270 | IGHV3- | IGHJ4 | 6 | 43 | 139 | 221 | 308 | 689 | 1197 |
| | 74 | | | | | | | | |
| set2_271 | IGHV5- | IGHJ4 | 17 | 61 | 157 | 229 | 326 | 690 | 1197 |
| | 51 | | | | | | | | |
| set2_272 | IGHV3- | IGHJ5 | 2 | 38 | 135 | 216 | 304 | 671 | 1197 |
| | 30 | | | | | | | | |
| set2_273 | IGHV3- | IGHJ4 | 2 | 39 | 135 | 218 | 304 | 691 | 1197 |
| | 33 | | | | | | | | |
| set2_274 | IGHV3- | IGHJ4 | 2 | 38 | 135 | 216 | 304 | 692 | 1197 |
| | 30-3 | | | | | | | | |
| set2_275 | IGHV3- | IGHJ4 | 2 | 39 | 135 | 216 | 304 | 693 | 1197 |
| | 30 | | | | | | | | |
| set2_276 | IGHV4- | IGHJ4 | 15 | 66 | 161 | 242 | 312 | 694 | 1197 |
| | 34 | | | | | | | | |
| set2_277 | IGHV3- | IGHJ4 | 2 | 38 | 135 | 216 | 304 | 695 | 1197 |
| | 30 | | | | | | | | |
| set2_278 | IGHV3- | IGHJ5 | 2 | 38 | 135 | 216 | 304 | 696 | 1214 |
| | 30 | | | | | | | | |
| set2_279 | IGHV3- | IGHJ3 | 2 | 38 | 135 | 216 | 304 | 697 | 1198 |
| | 30 | | | | | | | | |
| set2_280 | IGHV3- | IGHJ4 | 2 | 39 | 135 | 218 | 304 | 698 | 1197 |
| | 33 | | | | | | | | |
| set2_281 | IGHV3- | IGHJ4 | 2 | 39 | 135 | 218 | 304 | 699 | 1197 |
| | 33 | | | | | | | | |
| set2_282 | IGHV3- | IGHJ4 | 2 | 38 | 135 | 216 | 304 | 700 | 1197 |
| | 30 | | | | | | | | |
| set2_283 | IGHV3- | IGHJ4 | 12 | 38 | 146 | 254 | 304 | 701 | 1197 |
| | 23 | | | | | | | | |
| set2_284 | IGHV3- | IGHJ4 | 2 | 38 | 135 | 216 | 304 | 702 | 1197 |
| | 30 | | | | | | | | |
| set2_285 | IGHV3- | IGHJ4 | 2 | 39 | 135 | 218 | 304 | 703 | 1197 |
| | 33 | | | | | | | | |
| set2_286 | IGHV3- | IGHJ4 | 2 | 38 | 135 | 216 | 304 | 704 | 1197 |
| | 30 | | | | | | | | |
| set2_287 | IGHV3- | IGHJ6 | 2 | 39 | 135 | 218 | 304 | 705 | 1200 |
| | 33 | | | | | | | | |
| set2_288 | IGHV3- | IGHJ4 | 6 | 43 | 139 | 221 | 308 | 706 | 1197 |
| | 74 | | | | | | | | |
| set2_289 | IGHV3- | IGHJ6 | 2 | 39 | 135 | 218 | 304 | 707 | 1196 |
| | 33 | | | | | | | | |
| set2_290 | IGHV3- | IGHJ6 | 2 | 39 | 135 | 218 | 304 | 708 | 1196 |
| | 33 | | | | | | | | |
| set2_291 | IGHV3- | IGHJ4 | 2 | 38 | 187 | 216 | 304 | 709 | 1197 |
| | 30-3 | | | | | | | | |
| set2_292 | IGHV2- | IGHJ4 | 25 | 74 | 167 | 249 | 339 | 710 | 1197 |
| | 70 | | | | | | | | |
| set2_293 | IGHV3- | IGHJ6 | 7 | 58 | 169 | 251 | 341 | 711 | 1196 |
| | 21 | | | | | | | | |
| set2_294 | IGHV3- | IGHJ4 | 2 | 39 | 135 | 273 | 371 | 712 | 1197 |
| | 33 | | | | | | | | |
| set2_295 | IGHV3- | IGHJ4 | 28 | 103 | 178 | 260 | 356 | 713 | 1215 |
| | 49 | | | | | | | | |
| set2_296 | IGHV3- | IGHJ6 | 2 | 38 | 135 | 216 | 372 | 714 | 1196 |
| | 30-3 | | | | | | | | |
| set2_297 | IGHV5- | IGHJ4 | 17 | 61 | 157 | 229 | 326 | 715 | 1197 |
| | 51 | | | | | | | | |
| set2_298 | IGHV3- | IGHJ4 | 2 | 38 | 135 | 216 | 304 | 716 | 1197 |
| | 30 | | | | | | | | |
| set2_299 | IGHV3- | IGHJ5 | 2 | 101 | 135 | 274 | 373 | 717 | 1203 |
| | 30 | | | | | | | | |
| set2_300 | IGHV3- | IGHJ4 | 2 | 38 | 135 | 216 | 304 | 718 | 1197 |
| | 30 | | | | | | | | |
| set2_301 | IGHV3- | IGHJ4 | 2 | 38 | 135 | 216 | 304 | 719 | 1197 |
| | 30 | | | | | | | | |
| set2_302 | IGHV3- | IGHJ3 | 2 | 38 | 135 | 216 | 304 | 720 | 1198 |
| | 30 | | | | | | | | |
| set2_303 | IGHV3- | IGHJ4 | 2 | 38 | 135 | 216 | 304 | 721 | 1197 |
| | 30 | | | | | | | | |
| set2_304 | IGHV3- | IGHJ3 | 2 | 38 | 135 | 216 | 304 | 722 | 1198 |
| | 30-3 | | | | | | | | |
| set2_305 | IGHV3- | IGHJ6 | 2 | 38 | 135 | 216 | 304 | 723 | 1196 |
| | 30 | | | | | | | | |
| set2_306 | IGHV3- | IGHJ6 | 2 | 38 | 188 | 275 | 374 | 724 | 1196 |
| | 30 | | | | | | | | |
| set2_307 | IGHV3- | IGHJ3 | 2 | 38 | 135 | 216 | 304 | 725 | 1198 |
| | 30 | | | | | | | | |
| set2_308 | IGHV3- | IGHJ4 | 2 | 38 | 135 | 216 | 304 | 726 | 1197 |
| | 30 | | | | | | | | |
| set2_309 | IGHV1- | IGHJ5 | 30 | 98 | 184 | 267 | 364 | 727 | 1197 |
| | 24 | | | | | | | | |
| set2_310 | IGHV3- | IGHJ5 | 2 | 38 | 135 | 216 | 304 | 728 | 1197 |
| | 30 | | | | | | | | |
| set2_311 | IGHV1- | IGHJ5 | 30 | 98 | 184 | 267 | 364 | 729 | 1197 |
| | 24 | | | | | | | | |
| set2_312 | IGHV3- | IGHJ4 | 2 | 38 | 135 | 216 | 304 | 730 | 1197 |
| | 30 | | | | | | | | |
| set2_313 | IGHV3- | IGHJ6 | 2 | 104 | 141 | 276 | 375 | 731 | 1196 |
| | 30-3 | | | | | | | | |
| set2_314 | IGHV2- | IGHJ4 | 24 | 72 | 166 | 247 | 336 | 732 | 1197 |
| | 5 | | | | | | | | |
| set2_315 | IGHV3- | IGHJ3 | 2 | 38 | 135 | 216 | 304 | 733 | 1198 |
| | 30-3 | | | | | | | | |
| set2_316 | IGHV3- | IGHJ6 | 7 | 58 | 169 | 251 | 341 | 734 | 1196 |
| | 21 | | | | | | | | |
| set2_317 | IGHV3- | IGHJ3 | 2 | 38 | 135 | 216 | 304 | 735 | 1198 |
| | 30-3 | | | | | | | | |
| set2_318 | IGHV3- | IGHJ3 | 2 | 38 | 135 | 216 | 304 | 736 | 1198 |
| | 30 | | | | | | | | |
| set2_319 | IGHV3- | IGHJ4 | 2 | 38 | 135 | 216 | 304 | 737 | 1197 |
| | 30 | | | | | | | | |
| set2_320 | IGHV1- | IGHJ5 | 30 | 98 | 184 | 267 | 364 | 738 | 1197 |
| | 24 | | | | | | | | |
| set2_321 | IGHV3- | IGHJ5 | 2 | 38 | 135 | 216 | 304 | 739 | 1197 |
| | 30 | | | | | | | | |
| set2_322 | IGHV3- | IGHJ4 | 2 | 38 | 135 | 216 | 304 | 740 | 1197 |
| | 30-3 | | | | | | | | |
| set2_323 | IGHV3- | IGHJ3 | 2 | 38 | 135 | 216 | 304 | 741 | 1198 |
| | 30 | | | | | | | | |
| set2_324 | IGHV3- | IGHJ5 | 2 | 38 | 135 | 216 | 304 | 742 | 1197 |
| | 30-3 | | | | | | | | |
| set2_325 | IGHV3- | IGHJ6 | 2 | 38 | 135 | 216 | 304 | 743 | 1196 |
| | 30-3 | | | | | | | | |
| set2_326 | IGHV3- | IGHJ6 | 2 | 38 | 135 | 216 | 304 | 744 | 1196 |
| | 30 | | | | | | | | |
| set2_327 | IGHV3- | IGHJ4 | 2 | 38 | 135 | 216 | 304 | 745 | 1197 |
| | 30 | | | | | | | | |
| set2_328 | IGHV1- | IGHJ5 | 30 | 98 | 184 | 267 | 364 | 746 | 1197 |
| | 24 | | | | | | | | |
| set2_329 | IGHV3- | IGHJ4 | 2 | 39 | 135 | 216 | 304 | 747 | 1197 |
| | 30 | | | | | | | | |
| set2_330 | IGHV3- | IGHJ4 | 2 | 38 | 135 | 216 | 304 | 748 | 1197 |
| | 30 | | | | | | | | |
| set2_331 | IGHV3- | IGHJ4 | 6 | 43 | 171 | 253 | 343 | 749 | 1197 |
| | 7 | | | | | | | | |
| set2_332 | IGHV3- | IGHJ4 | 2 | 38 | 135 | 216 | 304 | 750 | 1197 |
| | 30-3 | | | | | | | | |
| set2_333 | IGHV3- | IGHJ4 | 2 | 38 | 135 | 216 | 304 | 751 | 1197 |
| | 30 | | | | | | | | |
| set2_334 | IGHV3- | IGHJ4 | 2 | 38 | 135 | 216 | 304 | 752 | 1197 |
| | 30 | | | | | | | | |
| set2_335 | IGHV3- | IGHJ6 | 2 | 38 | 135 | 216 | 304 | 753 | 1196 |
| | 30-3 | | | | | | | | |
| set2_336 | IGHV1- | IGHJ5 | 30 | 98 | 184 | 267 | 364 | 754 | 1197 |
| | 24 | | | | | | | | |
| set2_337 | IGHV3- | IGHJ5 | 2 | 38 | 135 | 216 | 304 | 755 | 1197 |
| | 30 | | | | | | | | |
| set2_338 | IGHV3- | IGHJ4 | 2 | 38 | 135 | 216 | 304 | 756 | 1197 |
| | 30 | | | | | | | | |
| set2_339 | IGHV3- | IGHJ4 | 6 | 43 | 171 | 253 | 343 | 757 | 1197 |
| | 7 | | | | | | | | |
| set2_340 | IGHV3- | IGHJ4 | 2 | 38 | 189 | 216 | 376 | 758 | 1197 |
| | 30 | | | | | | | | |
| set2_341 | IGHV3- | IGHJ4 | 2 | 38 | 135 | 216 | 304 | 759 | 1197 |
| | 30 | | | | | | | | |
| set2_342 | IGHV3- | IGHJ4 | 7 | 58 | 169 | 251 | 341 | 760 | 1197 |
| | 21 | | | | | | | | |
| set2_343 | IGHV3- | IGHJ6 | 2 | 39 | 135 | 218 | 304 | 761 | 1196 |
| | 33 | | | | | | | | |
| set2_344 | IGHV3- | IGHJ4 | 2 | 38 | 135 | 216 | 304 | 762 | 1197 |
| | 30-3 | | | | | | | | |
| set2_345 | IGHV3- | IGHJ5 | 2 | 38 | 135 | 216 | 304 | 763 | 1197 |
| | 30-3 | | | | | | | | |
| set2_346 | IGHV4- | IGHJ6 | 9 | 47 | 143 | 225 | 312 | 764 | 1200 |
| | 59 | | | | | | | | |
| set2_347 | IGHV1- | IGHJ5 | 30 | 98 | 184 | 267 | 364 | 765 | 1197 |
| | 24 | | | | | | | | |
| set2_348 | IGHV3- | IGHJ4 | 2 | 38 | 135 | 216 | 304 | 766 | 1197 |
| | 30 | | | | | | | | |
| set2_349 | IGHV3- | IGHJ6 | 2 | 101 | 135 | 216 | 304 | 767 | 1196 |
| | 30-3 | | | | | | | | |
| set2_350 | IGHV1- | IGHJ5 | 30 | 98 | 184 | 267 | 364 | 768 | 1197 |
| | 24 | | | | | | | | |
| set2_351 | IGHV3- | IGHJ3 | 2 | 38 | 135 | 216 | 304 | 769 | 1198 |
| | 30 | | | | | | | | |
| set2_352 | IGHV3- | IGHJ6 | 2 | 105 | 141 | 216 | 377 | 770 | 1196 |
| | 30 | | | | | | | | |
| set2_353 | IGHV3- | IGHJ4 | 2 | 39 | 135 | 218 | 304 | 771 | 1197 |
| | 33 | | | | | | | | |
| set2_354 | IGHV1- | IGHJ5 | 30 | 98 | 184 | 267 | 364 | 772 | 1197 |
| | 24 | | | | | | | | |
| set2_355 | IGHV1- | IGHJ5 | 30 | 98 | 184 | 267 | 364 | 773 | 1197 |
| | 24 | | | | | | | | |
| set2_356 | IGHV3- | IGHJ4 | 6 | 106 | 190 | 277 | 378 | 774 | 1197 |
| | 13 | | | | | | | | |
| set2_357 | IGHV3- | IGHJ4 | 2 | 38 | 135 | 278 | 379 | 775 | 1197 |
| | 30 | | | | | | | | |
| set2_358 | IGHV3- | IGHJ4 | 32 | 107 | 191 | 279 | 380 | 776 | 1197 |
| | 30 | | | | | | | | |
| set2_359 | IGHV3- | IGHJ3 | 2 | 38 | 135 | 216 | 304 | 777 | 1198 |
| | 30-3 | | | | | | | | |
| set2_360 | IGHV3- | IGHJ3 | 2 | 39 | 135 | 216 | 304 | 778 | 1198 |
| | 30 | | | | | | | | |
| set2_361 | IGHV3- | IGHJ4 | 2 | 38 | 135 | 216 | 304 | 779 | 1197 |
| | 30 | | | | | | | | |
| set2_362 | IGHV1- | IGHJ5 | 30 | 98 | 184 | 267 | 364 | 780 | 1197 |
| | 24 | | | | | | | | |
| set2_363 | IGHV1- | IGHJ5 | 30 | 98 | 184 | 267 | 364 | 781 | 1197 |
| | 24 | | | | | | | | |
| set2_364 | IGHV1- | IGHJ5 | 30 | 98 | 184 | 267 | 364 | 782 | 1197 |
| | 24 | | | | | | | | |
| set2_365 | IGHV1- | IGHJ5 | 30 | 98 | 184 | 267 | 364 | 783 | 1197 |
| | 24 | | | | | | | | |
| set2_366 | IGHV3- | IGHJ4 | 2 | 38 | 135 | 216 | 304 | 784 | 1197 |
| | 30-3 | | | | | | | | |
| set2_367 | IGHV3- | IGHJ4 | 2 | 38 | 135 | 216 | 304 | 785 | 1197 |
| | 30-3 | | | | | | | | |
| set2_368 | IGHV3- | IGHJ4 | 2 | 39 | 135 | 216 | 304 | 786 | 1197 |
| | 30 | | | | | | | | |
| set2_369 | IGHV2- | IGHJ4 | 25 | 74 | 167 | 249 | 339 | 787 | 1197 |
| | 70 | | | | | | | | |
| set2_370 | IGHV3- | IGHJ3 | 2 | 39 | 135 | 216 | 304 | 788 | 1198 |
| | 30 | | | | | | | | |
| set2_371 | IGHV3- | IGHJ4 | 2 | 38 | 135 | 216 | 304 | 789 | 1197 |
| | 30 | | | | | | | | |
| set2_372 | IGHV3- | IGHJ5 | 2 | 38 | 135 | 216 | 304 | 790 | 1197 |
| | 30-3 | | | | | | | | |
| set2_373 | IGHV3- | IGHJ4 | 2 | 38 | 135 | 216 | 304 | 791 | 1197 |
| | 30 | | | | | | | | |
| set2_374 | IGHV3- | IGHJ3 | 2 | 101 | 135 | 216 | 381 | 792 | 1198 |
| | 30 | | | | | | | | |
| set2_375 | IGHV3- | IGHJ2 | 6 | 106 | 190 | 280 | 378 | 793 | 1203 |
| | 13 | | | | | | | | |
| set2_376 | IGHV3- | IGHJ6 | 2 | 39 | 135 | 218 | 304 | 794 | 1196 |
| | 33 | | | | | | | | |
| set2_377 | IGHV3- | IGHJ4 | 2 | 39 | 135 | 216 | 304 | 795 | 1197 |
| | 30 | | | | | | | | |
| set2_378 | IGHV3- | IGHJ4 | 2 | 38 | 135 | 216 | 304 | 796 | 1197 |
| | 30 | | | | | | | | |
| set2_379 | IGHV3- | IGHJ6 | 6 | 43 | 171 | 253 | 343 | 797 | 1196 |
| | 7 | | | | | | | | |
| set2_380 | IGHV3- | IGHJ3 | 2 | 38 | 135 | 216 | 304 | 798 | 1198 |
| | 30 | | | | | | | | |
| set2_381 | IGHV3- | IGHJ4 | 2 | 38 | 135 | 216 | 304 | 799 | 1197 |
| | 30 | | | | | | | | |
| set2_382 | IGHV3- | IGHJ4 | 2 | 38 | 135 | 216 | 304 | 800 | 1197 |
| | 30 | | | | | | | | |
| set2_383 | IGHV3- | IGHJ4 | 2 | 38 | 135 | 216 | 304 | 801 | 1197 |
| | 30-3 | | | | | | | | |
| set2_384 | IGHV3- | IGHJ4 | 2 | 38 | 135 | 216 | 304 | 802 | 1197 |
| | 30 | | | | | | | | |
| set2_385 | IGHV3- | IGHJ4 | 2 | 38 | 135 | 216 | 304 | 803 | 1197 |
| | 30 | | | | | | | | |
| set2_386 | IGHV1- | IGHJ4 | 30 | 98 | 184 | 267 | 364 | 804 | 1197 |
| | 24 | | | | | | | | |
| set2_387 | IGHV3- | IGHJ6 | 2 | 38 | 135 | 216 | 304 | 805 | 1196 |
| | 30 | | | | | | | | |
| set2_388 | IGHV1- | IGHJ4 | 30 | 98 | 184 | 267 | 364 | 806 | 1197 |
| | 24 | | | | | | | | |
| set2_389 | IGHV3- | IGHJ4 | 2 | 38 | 135 | 216 | 304 | 807 | 1197 |
| | 30 | | | | | | | | |
| set2_390 | IGHV3- | IGHJ5 | 2 | 38 | 135 | 216 | 304 | 808 | 1197 |
| | 30-3 | | | | | | | | |
| set2_391 | IGHV3- | IGHJ4 | 2 | 38 | 135 | 216 | 304 | 809 | 1197 |
| | 30 | | | | | | | | |
| set2_392 | IGHV3- | IGHJ3 | 2 | 38 | 135 | 216 | 304 | 810 | 1198 |
| | 30 | | | | | | | | |
| set2_393 | IGHV2- | IGHJ4 | 33 | 74 | 192 | 249 | 339 | 811 | 1197 |
| | 70 | | | | | | | | |
| set2_394 | IGHV3- | IGHJ4 | 2 | 38 | 135 | 216 | 304 | 812 | 1197 |
| | 30 | | | | | | | | |
| set2_395 | IGHV3- | IGHJ6 | 2 | 39 | 135 | 218 | 304 | 813 | 1196 |
| | 33 | | | | | | | | |
| set2_396 | IGHV3- | IGHJ3 | 2 | 38 | 135 | 216 | 304 | 814 | 1198 |
| | 30-3 | | | | | | | | |
| set2_397 | IGHV3- | IGHJ3 | 2 | 38 | 135 | 216 | 304 | 815 | 1198 |
| | 30 | | | | | | | | |
| set2_398 | IGHV3- | IGHJ3 | 2 | 38 | 135 | 216 | 304 | 816 | 1198 |
| | 30 | | | | | | | | |
| set2_399 | IGHV3- | IGHJ4 | 2 | 38 | 135 | 216 | 304 | 817 | 1197 |
| | 30 | | | | | | | | |
| set2_400 | IGHV3- | IGHJ4 | 2 | 38 | 135 | 216 | 304 | 818 | 1197 |
| | 30 | | | | | | | | |
| set2_401 | IGHV1- | IGHJ5 | 30 | 98 | 184 | 267 | 364 | 819 | 1197 |
| | 24 | | | | | | | | |
| set2_402 | IGHV3- | IGHJ6 | 2 | 39 | 135 | 216 | 304 | 820 | 1196 |
| | 30 | | | | | | | | |
| set2_403 | IGHV3- | IGHJ4 | 7 | 58 | 169 | 251 | 341 | 821 | 1197 |
| | 21 | | | | | | | | |
| set2_404 | IGHV3- | IGHJ4 | 6 | 106 | 190 | 277 | 378 | 822 | 1197 |
| | 13 | | | | | | | | |
| set2_405 | IGHV3- | IGHJ5 | 2 | 38 | 135 | 216 | 304 | 823 | 1197 |
| | 30 | | | | | | | | |
| set2_406 | IGHV3- | IGHJ4 | 6 | 43 | 171 | 253 | 343 | 824 | 1197 |
| | 7 | | | | | | | | |
| set2_407 | IGHV3- | IGHJ4 | 2 | 38 | 135 | 216 | 304 | 825 | 1197 |
| | 30-3 | | | | | | | | |
| set2_408 | IGHV2- | IGHJ4 | 25 | 74 | 167 | 249 | 339 | 826 | 1197 |
| | 70 | | | | | | | | |
| set2_409 | IGHV3- | IGHJ4 | 2 | 38 | 135 | 216 | 304 | 827 | 1197 |
| | 30 | | | | | | | | |
| set2_410 | IGHV3- | IGHJ4 | 6 | 108 | 154 | 272 | 341 | 828 | 1197 |
| | 48 | | | | | | | | |
| set2_411 | IGHV3- | IGHJ5 | 2 | 38 | 135 | 216 | 304 | 829 | 1197 |
| | 30 | | | | | | | | |
| set2_412 | IGHV3- | IGHJ4 | 2 | 91 | 135 | 281 | 304 | 830 | 1197 |
| | 30 | | | | | | | | |
| set2_413 | IGHV1- | IGHJ5 | 30 | 98 | 184 | 267 | 364 | 831 | 1197 |
| | 24 | | | | | | | | |
| set2_414 | IGHV3- | IGHJ6 | 2 | 39 | 135 | 218 | 304 | 832 | 1196 |
| | 33 | | | | | | | | |
| set2_415 | IGHV3- | IGHJ4 | 2 | 38 | 135 | 216 | 382 | 833 | 1197 |
| | 30 | | | | | | | | |
| set2_416 | IGHV3- | IGHJ3 | 2 | 38 | 193 | 216 | 383 | 834 | 1198 |
| | 30-3 | | | | | | | | |
| set2_417 | IGHV3- | IGHJ5 | 2 | 38 | 135 | 216 | 304 | 835 | 1197 |
| | 30 | | | | | | | | |
| set2_418 | IGHV3- | IGHJ3 | 2 | 39 | 135 | 216 | 304 | 836 | 1198 |
| | 30 | | | | | | | | |
| set2_419 | IGHV3- | IGHJ4 | 12 | 38 | 146 | 254 | 304 | 837 | 1197 |
| | 23 | | | | | | | | |
| set2_420 | IGHV3- | IGHJ4 | 2 | 39 | 135 | 216 | 304 | 838 | 1197 |
| | 30 | | | | | | | | |
| set2_421 | IGHV3- | IGHJ5 | 2 | 38 | 135 | 216 | 304 | 839 | 1197 |
| | 30 | | | | | | | | |
| set2_422 | IGHV3- | IGHJI | 2 | 38 | 181 | 216 | 304 | 840 | 1197 |
| | 30 | | | | | | | | |
| set2_423 | IGHV1- | IGHJ4 | 14 | 70 | 164 | 245 | 334 | 841 | 1197 |
| | 18 | | | | | | | | |
| set2_424 | IGHV3- | IGHJ4 | 2 | 38 | 135 | 216 | 304 | 842 | 1197 |
| | 30-3 | | | | | | | | |
| set2_425 | IGHV4- | IGHJ3 | 8 | 109 | 194 | 225 | 355 | 843 | 1198 |
| | 31 | | | | | | | | |
| set2_426 | IGHV3- | IGHJ4 | 2 | 38 | 135 | 216 | 304 | 844 | 1197 |
| | 30 | | | | | | | | |
| set2_427 | IGHV3- | IGHJ4 | 7 | 110 | 169 | 251 | 341 | 845 | 1197 |
| | 21 | | | | | | | | |
| set2_428 | IGHV3- | IGHJ4 | 2 | 38 | 135 | 216 | 304 | 846 | 1197 |
| | 30-3 | | | | | | | | |
| set2_429 | IGHV3- | IGHJ6 | 2 | 39 | 135 | 216 | 304 | 847 | 1196 |
| | 30 | | | | | | | | |
| set2_430 | IGHV3- | IGHJ4 | 2 | 111 | 135 | 216 | 384 | 848 | 1197 |
| | 30-3 | | | | | | | | |
| set2_431 | IGHV3- | IGHJ4 | 2 | 38 | 135 | 216 | 304 | 849 | 1197 |
| | 30 | | | | | | | | |
| set2_432 | IGHV3- | IGHJ4 | 2 | 38 | 135 | 216 | 304 | 850 | 1197 |
| | 30 | | | | | | | | |
| set2_433 | IGHV3- | IGHJ4 | 12 | 38 | 146 | 254 | 304 | 851 | 1197 |
| | 23 | | | | | | | | |
| set2_434 | IGHV1- | IGHJ6 | 30 | 98 | 184 | 267 | 364 | 852 | 1200 |
| | 24 | | | | | | | | |
| set2_435 | IGHV3- | IGHJ5 | 2 | 38 | 135 | 216 | 304 | 853 | 1197 |
| | 30 | | | | | | | | |
| set2_436 | IGHV3- | IGHJ3 | 2 | 38 | 135 | 216 | 304 | 854 | 1198 |
| | 30 | | | | | | | | |
| set2_437 | IGHV3- | IGHJ6 | 2 | 38 | 135 | 216 | 385 | 855 | 1196 |
| | 30-3 | | | | | | | | |
| set2_438 | IGHV3- | IGHJ4 | 2 | 39 | 135 | 216 | 304 | 856 | 1197 |
| | 30 | | | | | | | | |
| set2_439 | IGHV3- | IGHJ5 | 2 | 38 | 135 | 216 | 304 | 857 | 1197 |
| | 30-3 | | | | | | | | |
| set2_440 | IGHV1- | IGHJ5 | 30 | 98 | 184 | 267 | 364 | 858 | 1197 |
| | 24 | | | | | | | | |
| set2_441 | IGHV3- | IGHJ4 | 2 | 38 | 135 | 216 | 304 | 859 | 1197 |
| | 30 | | | | | | | | |
| set2_442 | IGHV3- | IGHJ4 | 2 | 38 | 135 | 216 | 304 | 860 | 1197 |
| | 30-3 | | | | | | | | |
| set2_443 | IGHV3- | IGHJ6 | 2 | 38 | 135 | 216 | 304 | 861 | 1196 |
| | 30 | | | | | | | | |
| set2_444 | IGHV3- | IGHJ4 | 2 | 38 | 135 | 216 | 304 | 862 | 1197 |
| | 30-3 | | | | | | | | |
| set2_445 | IGHV3- | IGHJI | 2 | 112 | 195 | 216 | 386 | 863 | 1197 |
| | 30-3 | | | | | | | | |
| set2_446 | IGHV3- | IGHJ6 | 6 | 58 | 154 | 235 | 341 | 864 | 1196 |
| | 48 | | | | | | | | |
| set2_447 | IGHV3- | IGHJ4 | 18 | 92 | 158 | 238 | 361 | 865 | 1197 |
| | 9 | | | | | | | | |
| set2_448 | IGHV3- | IGHJ4 | 2 | 39 | 135 | 218 | 304 | 866 | 1197 |
| | 33 | | | | | | | | |
| set2_449 | IGHV3- | IGHJ6 | 2 | 39 | 135 | 216 | 304 | 867 | 1196 |
| | 30 | | | | | | | | |
| set2_450 | IGHV1- | IGHJ5 | 30 | 98 | 184 | 267 | 387 | 868 | 1197 |
| | 24 | | | | | | | | |
| set2_451 | IGHV1- | IGHJ5 | 30 | 98 | 184 | 267 | 364 | 869 | 1197 |
| | 24 | | | | | | | | |
| set2_452 | IGHV3- | IGHJ5 | 21 | 59 | 155 | 236 | 304 | 870 | 1197 |
| | 53 | | | | | | | | |
| set2_453 | IGHV3- | IGHJ4 | 2 | 39 | 135 | 216 | 304 | 871 | 1197 |
| | 30 | | | | | | | | |
| set2_454 | IGHV3- | IGHJ3 | 2 | 39 | 196 | 282 | 388 | 872 | 1198 |
| | 30 | | | | | | | | |
| set2_455 | IGHV3- | IGHJ4 | 2 | 38 | 135 | 216 | 304 | 873 | 1197 |
| | 30-3 | | | | | | | | |
| set2_456 | IGHV3- | IGHJ4 | 2 | 39 | 135 | 283 | 304 | 874 | 1197 |
| | 33 | | | | | | | | |
| set2_457 | IGHV3- | IGHJ4 | 2 | 38 | 135 | 216 | 304 | 875 | 1197 |
| | 30 | | | | | | | | |
| set2_458 | IGHV3- | IGHJ6 | 2 | 39 | 135 | 218 | 304 | 876 | 1196 |
| | 33 | | | | | | | | |
| set2_459 | IGHV3- | IGHJ6 | 2 | 38 | 135 | 216 | 304 | 877 | 1196 |
| | 30 | | | | | | | | |
| set2_460 | IGHV3- | IGHJ3 | 2 | 38 | 135 | 216 | 389 | 878 | 1197 |
| | 30 | | | | | | | | |
| set2_461 | IGHV3- | IGHJ3 | 2 | 38 | 135 | 216 | 304 | 879 | 1198 |
| | 30 | | | | | | | | |
| set2_462 | IGHV3- | IGHJ4 | 2 | 38 | 135 | 216 | 304 | 880 | 1197 |
| | 30 | | | | | | | | |
| set2_463 | IGHV3- | IGHJ4 | 2 | 38 | 135 | 216 | 304 | 881 | 1197 |
| | 30-3 | | | | | | | | |
| set2_464 | IGHV3- | IGHJ4 | 2 | 113 | 181 | 284 | 390 | 882 | 1197 |
| | 30-3 | | | | | | | | |
| set2_465 | IGHV3- | IGHJ6 | 2 | 38 | 135 | 216 | 304 | 883 | 1196 |
| | 30 | | | | | | | | |
| set2_466 | IGHV3- | IGHJ4 | 2 | 38 | 135 | 216 | 304 | 884 | 1197 |
| | 30 | | | | | | | | |
| set2_467 | IGHV3- | IGHJ4 | 34 | 38 | 197 | 285 | 391 | 885 | 1197 |
| | 30-3 | | | | | | | | |
| set2_468 | IGHV3- | IGHJ6 | 7 | 58 | 169 | 251 | 341 | 886 | 1196 |
| | 21 | | | | | | | | |
| set2_469 | IGHV3- | IGHJ3 | 2 | 111 | 193 | 216 | 304 | 887 | 1198 |
| | 30-3 | | | | | | | | |
| set2_470 | IGHV3- | IGHJ4 | 2 | 38 | 135 | 216 | 392 | 888 | 1197 |
| | 30 | | | | | | | | |
| set2_471 | IGHV3- | IGHJ4 | 2 | 39 | 135 | 216 | 304 | 889 | 1197 |
| | 30 | | | | | | | | |
| set2_472 | IGHV3- | IGHJ3 | 2 | 38 | 135 | 216 | 304 | 890 | 1198 |
| | 30 | | | | | | | | |
| set2_473 | IGHV1- | IGHJ4 | 30 | 98 | 184 | 267 | 364 | 891 | 1197 |
| | 24 | | | | | | | | |
| set2_474 | IGHV3- | IGHJ3 | 2 | 39 | 135 | 218 | 304 | 892 | 1198 |
| | 33 | | | | | | | | |
| set2_475 | IGHV3- | IGHJ6 | 2 | 39 | 135 | 216 | 304 | 893 | 1196 |
| | 30 | | | | | | | | |
| set2_476 | IGHV3- | IGHJ4 | 7 | 58 | 169 | 251 | 341 | 894 | 1197 |
| | 21 | | | | | | | | |
| set2_477 | IGHV3- | IGHJ4 | 6 | 114 | 139 | 286 | 393 | 895 | 1197 |
| | 74 | | | | | | | | |
| set2_478 | IGHV3- | IGHJ4 | 2 | 38 | 135 | 216 | 304 | 896 | 1197 |
| | 30 | | | | | | | | |
| set2_479 | IGHV3- | IGHJ4 | 21 | 59 | 155 | 236 | 304 | 897 | 1210 |
| | 53 | | | | | | | | |
| set2_480 | IGHV3- | IGHJ3 | 2 | 39 | 135 | 216 | 304 | 898 | 1198 |
| | 30 | | | | | | | | |
| set2_481 | IGHV3- | IGHJ6 | 21 | 115 | 183 | 236 | 304 | 899 | 1196 |
| | 66 | | | | | | | | |
| set2_482 | IGHV2- | IGHJ4 | 25 | 74 | 167 | 249 | 339 | 900 | 1202 |
| | 70 | | | | | | | | |
| set2_483 | IGHV3- | IGHJ2 | 6 | 106 | 190 | 280 | 378 | 901 | 1203 |
| | 13 | | | | | | | | |
| set2_484 | IGHV4- | IGHJ4 | 15 | 66 | 161 | 242 | 394 | 902 | 1197 |
| | 34 | | | | | | | | |
| set2_485 | IGHV3- | IGHJ4 | 7 | 58 | 169 | 251 | 341 | 903 | 1197 |
| | 21 | | | | | | | | |
| set2_486 | IGHV1- | IGHJ5 | 30 | 98 | 184 | 267 | 364 | 904 | 1197 |
| | 24 | | | | | | | | |
| set2_487 | IGHV3- | IGHJ4 | 7 | 58 | 169 | 251 | 341 | 905 | 1197 |
| | 21 | | | | | | | | |
| set2_488 | IGHV5- | IGHJ4 | 17 | 116 | 198 | 287 | 395 | 906 | 1197 |
| | 51 | | | | | | | | |
| set2_489 | IGHV1- | IGHJ5 | 30 | 98 | 184 | 267 | 364 | 907 | 1197 |
| | 24 | | | | | | | | |
| set2_490 | IGHV1- | IGHJ4 | 4 | 90 | 199 | 288 | 396 | 908 | 1197 |
| | 69 | | | | | | | | |
| set2_491 | IGHV3- | IGHJ4 | 2 | 39 | 135 | 216 | 304 | 909 | 1197 |
| | 30 | | | | | | | | |
| set2_492 | IGHV3- | IGHJ4 | 12 | 38 | 146 | 254 | 304 | 910 | 1197 |
| | 23 | | | | | | | | |
| set2_493 | IGHV3- | IGHJ4 | 7 | 58 | 169 | 251 | 341 | 911 | 1197 |
| | 21 | | | | | | | | |
| set2_494 | IGHV3- | IGHJ4 | 2 | 39 | 135 | 216 | 304 | 912 | 1197 |
| | 30 | | | | | | | | |
| set2_495 | IGHV3- | IGHJ6 | 2 | 39 | 135 | 218 | 304 | 913 | 1196 |
| | 33 | | | | | | | | |
| set2_496 | IGHV1- | IGHJ4 | 4 | 90 | 199 | 288 | 396 | 914 | 1197 |
| | 69 | | | | | | | | |
| set2_497 | IGHV3- | IGHJ4 | 7 | 58 | 169 | 251 | 341 | 915 | 1197 |
| | 21 | | | | | | | | |
| set2_498 | IGHV4- | IGHJ4 | 9 | 47 | 143 | 225 | 312 | 916 | 1197 |
| | 59 | | | | | | | | |
| set2_499 | IGHV3- | IGHJ5 | 2 | 39 | 135 | 218 | 304 | 917 | 1197 |
| | 33 | | | | | | | | |
| set2_500 | IGHV3- | IGHJ6 | 35 | 117 | 200 | 289 | 397 | 918 | 1196 |
| | 43 | | | | | | | | |
| set2_501 | IGHV3- | IGHJ5 | 7 | 58 | 169 | 251 | 341 | 919 | 1197 |
| | 21 | | | | | | | | |
| set2_502 | IGHV3- | IGHJ4 | 12 | 38 | 146 | 254 | 304 | 920 | 1197 |
| | 23 | | | | | | | | |
| set2_503 | IGHV3- | IGHJ4 | 12 | 38 | 146 | 254 | 304 | 921 | 1197 |
| | 23 | | | | | | | | |
| set2_504 | IGHV3- | IGHJ4 | 7 | 58 | 169 | 251 | 341 | 922 | 1197 |
| | 21 | | | | | | | | |
| set2_505 | IGHV3- | IGHJ4 | 2 | 39 | 135 | 216 | 304 | 923 | 1197 |
| | 30 | | | | | | | | |
| set2_506 | IGHV3- | IGHJ4 | 7 | 58 | 169 | 251 | 341 | 924 | 1197 |
| | 21 | | | | | | | | |
| set2_507 | IGHV1- | IGHJ4 | 14 | 84 | 148 | 230 | 351 | 925 | 1197 |
| | 2 | | | | | | | | |
| set2_508 | IGHV4- | IGHJ3 | 9 | 47 | 143 | 225 | 312 | 926 | 1198 |
| | 59 | | | | | | | | |
| set2_509 | IGHV3- | IGHJ3 | 2 | 39 | 135 | 218 | 304 | 927 | 1198 |
| | 33 | | | | | | | | |
| set2_510 | IGHV3- | IGHJ6 | 2 | 39 | 135 | 216 | 304 | 928 | 1196 |
| | 30 | | | | | | | | |
| set2_511 | IGHV1- | IGHJ6 | 14 | 76 | 170 | 252 | 342 | 929 | 1196 |
| | 46 | | | | | | | | |
| set2_512 | IGHV4- | IGHJ5 | 9 | 47 | 143 | 225 | 312 | 930 | 1197 |
| | 59 | | | | | | | | |
| set2_513 | IGHV4- | IGHJ6 | 15 | 66 | 161 | 242 | 312 | 931 | 1196 |
| | 34 | | | | | | | | |
| set2_514 | IGHV1- | IGHJ4 | 14 | 76 | 170 | 252 | 342 | 932 | 1197 |
| | 46 | | | | | | | | |
| set2_515 | IGHV2- | IGHJ4 | 24 | 72 | 166 | 290 | 336 | 933 | 1197 |
| | 5 | | | | | | | | |
| set2_516 | IGHV4- | IGHJ6 | 9 | 118 | 143 | 225 | 312 | 934 | 1200 |
| | 59 | | | | | | | | |
| set2_517 | IGHV3- | IGHJ6 | 18 | 92 | 158 | 238 | 361 | 935 | 1196 |
| | 9 | | | | | | | | |
| set2_518 | IGHV3- | IGHJ4 | 2 | 39 | 201 | 216 | 304 | 936 | 1197 |
| | 30 | | | | | | | | |
| set2_519 | IGHV3- | IGHJ4 | 2 | 39 | 135 | 218 | 304 | 937 | 1197 |
| | 33 | | | | | | | | |
| set2_520 | IGHV3- | IGHJ6 | 6 | 43 | 171 | 253 | 343 | 938 | 1196 |
| | 7 | | | | | | | | |
| set2_521 | IGHV3- | IGHJ5 | 20 | 65 | 186 | 272 | 341 | 939 | 1197 |
| | 11 | | | | | | | | |
| set2_522 | IGHV1- | IGHJ4 | 14 | 70 | 164 | 245 | 334 | 940 | 1197 |
| | 18 | | | | | | | | |
| set2_523 | IGHV3- | IGHJ5 | 12 | 38 | 146 | 254 | 304 | 941 | 1197 |
| | 23 | | | | | | | | |
| set2_524 | IGHV3- | IGHJ4 | 2 | 38 | 135 | 216 | 304 | 942 | 1197 |
| | 30 | | | | | | | | |
| set2_525 | IGHV3- | IGHJ5 | 2 | 39 | 135 | 216 | 304 | 943 | 1197 |
| | 30 | | | | | | | | |
| set2_526 | IGHV3- | IGHJ5 | 2 | 38 | 202 | 216 | 304 | 944 | 1197 |
| | 30-3 | | | | | | | | |
| set2_527 | IGHV1- | IGHJ6 | 4 | 119 | 179 | 261 | 357 | 945 | 1196 |
| | 69 | | | | | | | | |
| set2_528 | IGHV3- | IGHJ4 | 12 | 38 | 146 | 254 | 304 | 946 | 1197 |
| | 23 | | | | | | | | |
| set2_529 | IGHV3- | IGHJ4 | 7 | 58 | 169 | 251 | 341 | 947 | 1197 |
| | 21 | | | | | | | | |
| set2_530 | IGHV1- | IGHJ6 | 30 | 98 | 184 | 267 | 364 | 948 | 1196 |
| | 24 | | | | | | | | |
| set2_531 | IGHV3- | IGHJ5 | 18 | 92 | 158 | 238 | 361 | 949 | 1197 |
| | 9 | | | | | | | | |
| set2_532 | IGHV4- | IGHJ6 | 9 | 47 | 143 | 225 | 312 | 950 | 1196 |
| | 59 | | | | | | | | |
| set2_533 | IGHV3- | IGHJ4 | 2 | 39 | 135 | 218 | 304 | 951 | 1197 |
| | 33 | | | | | | | | |
| set2_534 | IGHV3- | IGHJ4 | 2 | 39 | 135 | 218 | 304 | 952 | 1197 |
| | 33 | | | | | | | | |
| set2_535 | IGHV1- | IGHJ5 | 30 | 98 | 184 | 267 | 364 | 953 | 1197 |
| | 24 | | | | | | | | |
| set2_536 | IGHV3- | IGHJ4 | 2 | 39 | 135 | 216 | 304 | 954 | 1197 |
| | 30 | | | | | | | | |
| set2_537 | IGHV3- | IGHJ6 | 7 | 58 | 169 | 251 | 341 | 955 | 1196 |
| | 21 | | | | | | | | |
| set2_538 | IGHV3- | IGHJ6 | 2 | 38 | 135 | 216 | 304 | 956 | 1196 |
| | 30-3 | | | | | | | | |
| set2_539 | IGHV3- | IGHJ4 | 2 | 39 | 135 | 218 | 304 | 957 | 1197 |
| | 33 | | | | | | | | |
| set2_540 | IGHV3- | IGHJ6 | 2 | 39 | 135 | 216 | 304 | 958 | 1200 |
| | 30 | | | | | | | | |
| set2_541 | IGHV1- | IGHJ6 | 14 | 84 | 148 | 230 | 351 | 959 | 1196 |
| | 2 | | | | | | | | |
| set2_542 | IGHV3- | IGHJ4 | 36 | 39 | 203 | 291 | 304 | 960 | 1197 |
| | 30 | | | | | | | | |
| set2_543 | IGHV3- | IGHJ4 | 2 | 39 | 135 | 216 | 304 | 961 | 1197 |
| | 30 | | | | | | | | |
| set2_544 | IGHV3- | IGHJ4 | 2 | 39 | 135 | 218 | 304 | 962 | 1197 |
| | 33 | | | | | | | | |
| set2_545 | IGHV3- | IGHJ5 | 2 | 39 | 135 | 218 | 304 | 963 | 1197 |
| | 33 | | | | | | | | |
| set2_546 | IGHV1- | IGHJ5 | 30 | 98 | 184 | 267 | 364 | 964 | 1197 |
| | 24 | | | | | | | | |
| set2_547 | IGHV3- | IGHJ4 | 2 | 38 | 135 | 216 | 304 | 965 | 1197 |
| | 30 | | | | | | | | |
| set2_548 | IGHV1- | IGHJ6 | 4 | 90 | 199 | 288 | 396 | 966 | 1196 |
| | 69 | | | | | | | | |
| set2_549 | IGHV3- | IGHJ3 | 21 | 59 | 155 | 236 | 304 | 967 | 1198 |
| | 53 | | | | | | | | |
| set2_550 | IGHV1- | IGHJ4 | 30 | 98 | 184 | 267 | 364 | 968 | 1197 |
| | 24 | | | | | | | | |
| set2_551 | IGHV1- | IGHJ4 | 4 | 90 | 179 | 261 | 357 | 969 | 1197 |
| | 69 | | | | | | | | |
| set2_552 | IGHV3- | IGHJ6 | 2 | 39 | 135 | 216 | 304 | 970 | 1196 |
| | 30 | | | | | | | | |
| set2_553 | IGHV4- | IGHJ4 | 9 | 47 | 143 | 225 | 312 | 971 | 1197 |
| | 59 | | | | | | | | |
| set2_554 | IGHV3- | IGHJ3 | 12 | 38 | 146 | 254 | 304 | 972 | 1198 |
| | 23 | | | | | | | | |
| set2_555 | IGHV3- | IGHJ4 | 2 | 39 | 135 | 218 | 304 | 973 | 1197 |
| | 33 | | | | | | | | |
| set2_556 | IGHV4- | IGHJ4 | 11 | 88 | 145 | 225 | 355 | 974 | 1197 |
| | 39 | | | | | | | | |
| set2_557 | IGHV3- | IGHJ4 | 6 | 106 | 190 | 277 | 378 | 975 | 1197 |
| | 13 | | | | | | | | |
| set2_558 | IGHV3- | IGHJ3 | 12 | 38 | 146 | 254 | 304 | 976 | 1198 |
| | 23 | | | | | | | | |
| set2_559 | IGHV1- | IGHJ4 | 14 | 76 | 170 | 252 | 342 | 977 | 1197 |
| | 46 | | | | | | | | |
| set2_560 | IGHV3- | IGHJ4 | 2 | 39 | 135 | 216 | 304 | 978 | 1197 |
| | 30 | | | | | | | | |
| set2_561 | IGHV1- | IGHJ3 | 31 | 100 | 185 | 270 | 367 | 979 | 1198 |
| | 58 | | | | | | | | |
| set2_562 | IGHV3- | IGHJ6 | 6 | 58 | 154 | 235 | 324 | 980 | 1196 |
| | 48 | | | | | | | | |
| set2_563 | IGHV3- | IGHJ6 | 2 | 39 | 135 | 218 | 304 | 981 | 1196 |
| | 33 | | | | | | | | |
| set2_564 | IGHV4- | IGHJ4 | 9 | 47 | 143 | 225 | 312 | 982 | 1197 |
| | 59 | | | | | | | | |
| set2_565 | IGHV4- | IGHJ4 | 8 | 109 | 194 | 225 | 355 | 983 | 1197 |
| | 31 | | | | | | | | |
| set2_566 | IGHV3- | IGHJ4 | 7 | 120 | 204 | 292 | 398 | 984 | 1216 |
| | 21 | | | | | | | | |
| set2_567 | IGHV1- | IGHJ5 | 30 | 98 | 184 | 267 | 364 | 985 | 1197 |
| | 24 | | | | | | | | |
| set2_568 | IGHV4- | IGHJ3 | 11 | 88 | 145 | 225 | 355 | 986 | 1198 |
| | 39 | | | | | | | | |
| set2_569 | IGHV3- | IGHJ6 | 6 | 106 | 190 | 277 | 378 | 987 | 1196 |
| | 13 | | | | | | | | |
| set2_570 | IGHV2- | IGHJ3 | 33 | 74 | 192 | 249 | 339 | 988 | 1198 |
| | 70 | | | | | | | | |
| set2_571 | IGHV2- | IGHJ3 | 24 | 72 | 166 | 247 | 336 | 989 | 1198 |
| | 5 | | | | | | | | |
| set2_572 | IGHV3- | IGHJ6 | 6 | 58 | 154 | 235 | 341 | 990 | 1196 |
| | 48 | | | | | | | | |
| set2_573 | IGHV4- | IGHJ3 | 8 | 109 | 194 | 225 | 355 | 991 | 1198 |
| | 31 | | | | | | | | |
| set2_574 | IGHV4- | IGHJ4 | 8 | 109 | 194 | 225 | 355 | 992 | 1197 |
| | 31 | | | | | | | | |
| set2_575 | IGHV4- | IGHJ6 | 11 | 88 | 145 | 225 | 355 | 993 | 1196 |
| | 39 | | | | | | | | |
| set2_576 | IGHV1- | IGHJ6 | 4 | 90 | 199 | 288 | 396 | 994 | 1196 |
| | 69 | | | | | | | | |
| set2_577 | IGHV1- | IGHJ6 | 4 | 90 | 199 | 288 | 396 | 995 | 1196 |
| | 69 | | | | | | | | |
| set2_578 | IGHV3- | IGHJ4 | 2 | 39 | 135 | 218 | 304 | 996 | 1197 |
| | 33 | | | | | | | | |
| set2_579 | IGHV1- | IGHJ4 | 30 | 98 | 184 | 267 | 364 | 997 | 1197 |
| | 24 | | | | | | | | |
| set2_580 | IGHV1- | IGHJ6 | 4 | 90 | 199 | 288 | 396 | 998 | 1200 |
| | 69 | | | | | | | | |
| set2_581 | IGHV2- | IGHJ5 | 24 | 72 | 166 | 247 | 336 | 999 | 1197 |
| | 5 | | | | | | | | |
| set2_582 | IGHV1- | IGHJ5 | 14 | 84 | 148 | 230 | 351 | 1000 | 1197 |
| | 2 | | | | | | | | |
| set2_583 | IGHV3- | IGHJ6 | 6 | 43 | 171 | 253 | 343 | 1001 | 1196 |
| | 7 | | | | | | | | |
| set2_584 | IGHV3- | IGHJ3 | 6 | 59 | 155 | 236 | 338 | 1002 | 1198 |
| | 53 | | | | | | | | |
| set2_585 | IGHV2- | IGHJ3 | 25 | 74 | 167 | 249 | 339 | 1003 | 1198 |
| | 70 | | | | | | | | |
| set2_586 | IGHV1- | IGHJ4 | 30 | 98 | 184 | 267 | 364 | 1004 | 1197 |
| | 24 | | | | | | | | |
| set2_587 | IGHV1- | IGHJ4 | 30 | 98 | 184 | 267 | 364 | 1005 | 1197 |
| | 24 | | | | | | | | |
| set2_588 | IGHV4- | IGHJ4 | 9 | 47 | 143 | 225 | 312 | 1006 | 1197 |
| | 59 | | | | | | | | |
| set2_589 | IGHV4- | IGHJ3 | 11 | 88 | 145 | 225 | 355 | 1007 | 1198 |
| | 39 | | | | | | | | |
| set2_590 | IGHV1- | IGHJ5 | 30 | 98 | 184 | 267 | 364 | 1008 | 1197 |
| | 24 | | | | | | | | |
| set2_591 | IGHV3- | IGHJ6 | 2 | 39 | 135 | 293 | 304 | 1009 | 1196 |
| | 30 | | | | | | | | |
| set2_592 | IGHV1- | IGHJ6 | 4 | 90 | 199 | 288 | 396 | 1010 | 1196 |
| | 69 | | | | | | | | |
| set2_593 | IGHV3- | IGHJ4 | 20 | 65 | 186 | 272 | 341 | 1011 | 1197 |
| | 11 | | | | | | | | |
| set2_594 | IGHV2- | IGHJ4 | 33 | 74 | 192 | 249 | 339 | 1012 | 1197 |
| | 70 | | | | | | | | |
| set2_595 | IGHV2- | IGHJ4 | 25 | 74 | 167 | 249 | 339 | 1013 | 1197 |
| | 70 | | | | | | | | |
| set2_596 | IGHV3- | IGHJ6 | 12 | 38 | 146 | 254 | 304 | 1014 | 1196 |
| | 23 | | | | | | | | |
| set2_597 | IGHV1- | IGHJ4 | 4 | 90 | 205 | 294 | 399 | 1015 | 1197 |
| | 69 | | | | | | | | |
| set2_598 | IGHV3- | IGHJ6 | 6 | 43 | 171 | 253 | 343 | 1016 | 1196 |
| | 7 | | | | | | | | |
| set2_599 | IGHV1- | IGHJ6 | 14 | 76 | 170 | 252 | 342 | 1017 | 1196 |
| | 46 | | | | | | | | |
| set2_600 | IGHV3- | IGHJ6 | 20 | 65 | 186 | 272 | 341 | 1018 | 1196 |
| | 11 | | | | | | | | |
| set2_601 | IGHV4- | IGHJI | 15 | 66 | 161 | 242 | 312 | 1019 | 1197 |
| | 34 | | | | | | | | |
| set2_602 | IGHV3- | IGHJ6 | 7 | 58 | 169 | 251 | 341 | 1020 | 1196 |
| | 21 | | | | | | | | |
| set2_603 | IGHV3- | IGHJ6 | 6 | 43 | 171 | 253 | 343 | 1021 | 1196 |
| | 7 | | | | | | | | |
| set2_604 | IGHV3- | IGHJ6 | 6 | 43 | 171 | 253 | 343 | 1022 | 1196 |
| | 7 | | | | | | | | |
| set2_605 | IGHV4- | IGHJ5 | 15 | 66 | 161 | 242 | 312 | 1023 | 1197 |
| | 34 | | | | | | | | |
| set2_606 | IGHV4- | IGHJ6 | 15 | 66 | 161 | 242 | 312 | 1024 | 1196 |
| | 34 | | | | | | | | |
| set2_607 | IGHV2- | IGHJ4 | 29 | 94 | 182 | 264 | 360 | 1025 | 1197 |
| | 26 | | | | | | | | |
| set2_608 | IGHV4- | IGHJ5 | 9 | 121 | 143 | 225 | 312 | 1026 | 1197 |
| | 59 | | | | | | | | |
| set2_609 | IGHV1- | IGHJ6 | 4 | 71 | 199 | 288 | 357 | 1027 | 1196 |
| | 69 | | | | | | | | |
| set2_610 | IGHV3- | IGHJ6 | 12 | 38 | 146 | 254 | 304 | 1028 | 1196 |
| | 23 | | | | | | | | |
| set2_611 | IGHV3- | IGHJ6 | 6 | 43 | 171 | 253 | 343 | 1029 | 1196 |
| | 7 | | | | | | | | |
| set2_612 | IGHV3- | IGHJ4 | 2 | 38 | 135 | 216 | 304 | 1030 | 1197 |
| | 30 | | | | | | | | |
| set2_613 | IGHV4- | IGHJ5 | 15 | 122 | 161 | 242 | 400 | 1031 | 1197 |
| | 34 | | | | | | | | |
| set2_614 | IGHV3- | IGHJ6 | 20 | 65 | 186 | 272 | 341 | 1032 | 1196 |
| | 11 | | | | | | | | |
| set2_615 | IGHV4- | IGHJ3 | 15 | 66 | 161 | 242 | 312 | 1033 | 1198 |
| | 34 | | | | | | | | |
| set2_616 | IGHV4- | IGHJ5 | 15 | 66 | 161 | 242 | 312 | 1034 | 1197 |
| | 34 | | | | | | | | |
| set2_617 | IGHV1- | IGHJ5 | 30 | 98 | 184 | 267 | 364 | 1035 | 1197 |
| | 24 | | | | | | | | |
| set2_618 | IGHV1- | IGHJ6 | 14 | 76 | 170 | 252 | 342 | 1036 | 1196 |
| | 46 | | | | | | | | |
| set2_619 | IGHV1- | IGHJ6 | 14 | 76 | 170 | 252 | 342 | 1037 | 1196 |
| | 46 | | | | | | | | |
| set2_620 | IGHV1- | IGHJ3 | 14 | 84 | 148 | 230 | 351 | 1038 | 1198 |
| | 2 | | | | | | | | |
| set2_621 | IGHV4- | IGHJ5 | 15 | 66 | 161 | 242 | 312 | 1039 | 1197 |
| | 34 | | | | | | | | |
| set2_622 | IGHV1- | IGHJ6 | 4 | 123 | 206 | 295 | 357 | 1040 | 1196 |
| | 69 | | | | | | | | |
| set2_623 | IGHV3- | IGHJ6 | 6 | 43 | 171 | 253 | 343 | 1041 | 1196 |
| | 7 | | | | | | | | |
| set2_624 | IGHV4- | IGHJ6 | 15 | 66 | 161 | 242 | 312 | 1042 | 1196 |
| | 34 | | | | | | | | |
| set2_625 | IGHV3- | IGHJ6 | 12 | 38 | 146 | 254 | 304 | 1043 | 1196 |
| | 23 | | | | | | | | |
| set2_626 | IGHV3- | IGHJ6 | 6 | 43 | 171 | 253 | 343 | 1044 | 1196 |
| | 7 | | | | | | | | |
| set2_627 | IGHV3- | IGHJ6 | 2 | 39 | 135 | 218 | 304 | 1045 | 1196 |
| | 33 | | | | | | | | |
| set2_628 | IGHV5- | IGHJ4 | 37 | 61 | 207 | 296 | 401 | 1046 | 1197 |
| | 10-1 | | | | | | | | |
| set2_629 | IGHV3- | IGHJ6 | 20 | 65 | 186 | 272 | 341 | 1047 | 1196 |
| | 11 | | | | | | | | |
| set2_630 | IGHV4- | IGHJ5 | 11 | 124 | 208 | 297 | 402 | 1048 | 1197 |
| | 39 | | | | | | | | |
| set2_631 | IGHV5- | IGHJ4 | 37 | 61 | 207 | 296 | 401 | 1049 | 1197 |
| | 10-1 | | | | | | | | |
| set2_632 | IGHV3- | IGHJ6 | 6 | 43 | 171 | 253 | 343 | 1050 | 1196 |
| | 7 | | | | | | | | |
| set2_633 | IGHV3- | IGHJ4 | 7 | 44 | 140 | 222 | 309 | 1051 | 1197 |
| | 15 | | | | | | | | |
| set2_634 | IGHV3- | IGHJ4 | 6 | 106 | 190 | 277 | 378 | 1052 | 1197 |
| | 13 | | | | | | | | |
| set2_635 | IGHV3- | IGHJ6 | 6 | 125 | 171 | 253 | 403 | 1053 | 1217 |
| | 7 | | | | | | | | |
| set2_636 | IGHV2- | IGHJ4 | 29 | 94 | 182 | 264 | 360 | 1054 | 1197 |
| | 26 | | | | | | | | |
| set2_637 | IGHV3- | IGHJ6 | 12 | 38 | 146 | 254 | 304 | 1055 | 1196 |
| | 23 | | | | | | | | |
| set2_638 | IGHV3- | IGHJ6 | 20 | 65 | 186 | 272 | 341 | 1056 | 1196 |
| | 11 | | | | | | | | |
| set2_639 | IGHV3- | IGHJ6 | 20 | 65 | 186 | 272 | 341 | 1057 | 1196 |
| | 11 | | | | | | | | |
| set2_640 | IGHV4- | IGHJ5 | 15 | 66 | 161 | 242 | 312 | 1058 | 1197 |
| | 34 | | | | | | | | |
| set2_641 | IGHV1- | IGHJ4 | 14 | 70 | 164 | 245 | 334 | 1059 | 1197 |
| | 18 | | | | | | | | |
| set2_642 | IGHV4- | IGHJ4 | 11 | 88 | 145 | 225 | 355 | 1060 | 1197 |
| | 39 | | | | | | | | |
| set2_643 | IGHV1- | IGHJ6 | 14 | 84 | 148 | 230 | 351 | 1061 | 1196 |
| | 2 | | | | | | | | |
| set2_644 | IGHV4- | IGHJ5 | 15 | 66 | 161 | 242 | 312 | 1062 | 1197 |
| | 34 | | | | | | | | |
| set2_645 | IGHV4- | IGHJ4 | 15 | 66 | 161 | 242 | 312 | 1063 | 1197 |
| | 34 | | | | | | | | |
| set2_646 | IGHV1- | IGHJ6 | 4 | 90 | 199 | 288 | 404 | 1064 | 1200 |
| | 69 | | | | | | | | |
| set2_647 | IGHV1- | IGHJ6 | 4 | 90 | 199 | 288 | 396 | 1065 | 1196 |
| | 69 | | | | | | | | |
| set2_648 | IGHV3- | IGHJ6 | 7 | 58 | 169 | 251 | 341 | 1066 | 1196 |
| | 21 | | | | | | | | |
| set2_649 | IGHV4- | IGHJ6 | 15 | 66 | 161 | 242 | 405 | 1067 | 1196 |
| | 34 | | | | | | | | |
| set2_650 | IGHV3- | IGHJ6 | 2 | 39 | 135 | 218 | 304 | 1068 | 1196 |
| | 33 | | | | | | | | |
| set2_651 | IGHV3- | IGHJ4 | 7 | 58 | 169 | 251 | 341 | 1069 | 1197 |
| | 21 | | | | | | | | |
| set2_652 | IGHV5- | IGHJ5 | 37 | 61 | 207 | 296 | 401 | 1070 | 1197 |
| | 10-1 | | | | | | | | |
| set2_653 | IGHV3- | IGHJ6 | 6 | 43 | 171 | 253 | 343 | 1071 | 1196 |
| | 7 | | | | | | | | |
| set2_654 | IGHV1- | IGHJ6 | 4 | 90 | 199 | 261 | 357 | 1072 | 1196 |
| | 69 | | | | | | | | |
| set2_655 | IGHV3- | IGHJ4 | 2 | 39 | 135 | 216 | 304 | 1073 | 1197 |
| | 30 | | | | | | | | |
| set2_656 | IGHV3- | IGHJ4 | 7 | 58 | 169 | 251 | 341 | 1074 | 1197 |
| | 21 | | | | | | | | |
| set2_657 | IGHV3- | IGHJ6 | 6 | 43 | 171 | 253 | 343 | 1075 | 1196 |
| | 7 | | | | | | | | |
| set2_658 | IGHV4- | IGHJ6 | 15 | 66 | 209 | 298 | 406 | 1076 | 1196 |
| | 34 | | | | | | | | |
| set2_659 | IGHV3- | IGHJ4 | 2 | 39 | 135 | 216 | 304 | 1077 | 1197 |
| | 30 | | | | | | | | |
| set2_660 | IGHV1- | IGHJ6 | 4 | 90 | 199 | 288 | 396 | 1078 | 1200 |
| | 69 | | | | | | | | |
| set2_661 | IGHV4- | IGHJ3 | 15 | 66 | 161 | 242 | 312 | 1079 | 1198 |
| | 34 | | | | | | | | |
| set2_662 | IGHV3- | IGHJ6 | 12 | 38 | 146 | 254 | 304 | 1080 | 1196 |
| | 23 | | | | | | | | |
| set2_663 | IGHV1- | IGHJ6 | 4 | 90 | 199 | 288 | 396 | 1081 | 1196 |
| | 69 | | | | | | | | |
| set2_664 | IGHV3- | IGHJ4 | 2 | 39 | 135 | 216 | 304 | 1082 | 1197 |
| | 30 | | | | | | | | |
| set2_665 | IGHV3- | IGHJ6 | 12 | 38 | 146 | 254 | 304 | 1083 | 1196 |
| | 23 | | | | | | | | |
| set2_666 | IGHV1- | IGHJ5 | 30 | 98 | 184 | 267 | 364 | 1084 | 1197 |
| | 24 | | | | | | | | |
| set2_667 | IGHV3- | IGHJ3 | 7 | 44 | 140 | 222 | 309 | 1085 | 1198 |
| | 15 | | | | | | | | |
| set2_668 | IGHV4- | IGHJ5 | 11 | 88 | 145 | 225 | 355 | 1086 | 1197 |
| | 39 | | | | | | | | |
| set2_669 | IGHV4- | IGHJ3 | 9 | 126 | 210 | 225 | 407 | 1087 | 1198 |
| | 59 | | | | | | | | |
| set2_670 | IGHV4- | IGHJ6 | 15 | 66 | 161 | 242 | 312 | 1088 | 1196 |
| | 34 | | | | | | | | |
| set2_671 | IGHV4- | IGHJ5 | 9 | 47 | 143 | 225 | 312 | 1089 | 1197 |
| | 59 | | | | | | | | |
| set2_672 | IGHV4- | IGHJ5 | 15 | 66 | 161 | 242 | 312 | 1090 | 1197 |
| | 34 | | | | | | | | |
| set2_673 | IGHV1- | IGHJ5 | 30 | 98 | 184 | 267 | 364 | 1091 | 1197 |
| | 24 | | | | | | | | |
| set2_674 | IGHV4- | IGHJ4 | 11 | 88 | 145 | 225 | 355 | 1092 | 1197 |
| | 39 | | | | | | | | |
| set2_675 | IGHV4- | IGHJ3 | 9 | 47 | 143 | 299 | 312 | 1093 | 1198 |
| | 59 | | | | | | | | |
| set2_676 | IGHV4- | IGHJ6 | 9 | 47 | 211 | 225 | 408 | 1094 | 1218 |
| | 59 | | | | | | | | |
| set2_677 | IGHV1- | IGHJ6 | 14 | 70 | 164 | 245 | 334 | 1095 | 1196 |
| | 18 | | | | | | | | |
| set2_678 | IGHV2- | IGHJ4 | 29 | 94 | 182 | 264 | 360 | 1096 | 1197 |
| | 26 | | | | | | | | |
| set2_679 | IGHV3- | IGHJ6 | 12 | 38 | 146 | 254 | 304 | 1097 | 1196 |
| | 23 | | | | | | | | |
| set2_680 | IGHV1- | IGHJ6 | 4 | 90 | 199 | 288 | 396 | 1098 | 1200 |
| | 69 | | | | | | | | |
| set2_681 | IGHV4- | IGHJ3 | 9 | 47 | 175 | 256 | 347 | 1099 | 1198 |
| | 4 | | | | | | | | |
| set2_682 | IGHV3- | IGHJ3 | 7 | 44 | 140 | 222 | 309 | 1100 | 1198 |
| | 15 | | | | | | | | |
| set2_683 | IGHV1- | IGHJ6 | 14 | 84 | 148 | 230 | 351 | 1101 | 1196 |
| | 2 | | | | | | | | |
| set2_684 | IGHV3- | IGHJ4 | 2 | 39 | 135 | 216 | 304 | 1102 | 1197 |
| | 30 | | | | | | | | |
| set2_685 | IGHV1- | IGHJ6 | 4 | 90 | 199 | 288 | 396 | 1103 | 1200 |
| | 69 | | | | | | | | |
| set2_686 | IGHV4- | IGHJ5 | 8 | 109 | 194 | 225 | 355 | 1104 | 1197 |
| | 31 | | | | | | | | |
| set2_687 | IGHV3- | IGHJ5 | 6 | 127 | 139 | 300 | 308 | 1105 | 1197 |
| | 74 | | | | | | | | |
| set2_688 | IGHV1- | IGHJ5 | 30 | 98 | 184 | 267 | 364 | 1106 | 1197 |
| | 24 | | | | | | | | |
| set2_689 | IGHV1- | IGHJ5 | 4 | 128 | 199 | 288 | 396 | 1107 | 1197 |
| | 69 | | | | | | | | |
| set2_690 | IGHV5- | IGHJ4 | 17 | 61 | 157 | 229 | 326 | 1108 | 1197 |
| | 51 | | | | | | | | |
| set2_691 | IGHV1- | IGHJ6 | 14 | 70 | 164 | 245 | 334 | 1109 | 1196 |
| | 18 | | | | | | | | |
| set2_692 | IGHV1- | IGHJ5 | 14 | 70 | 164 | 245 | 334 | 1110 | 1197 |
| | 18 | | | | | | | | |
| set2_693 | IGHV3- | IGHJ6 | 20 | 65 | 186 | 272 | 341 | 1111 | 1196 |
| | 11 | | | | | | | | |
| set2_694 | IGHV3- | IGHJ6 | 12 | 38 | 146 | 254 | 304 | 1112 | 1196 |
| | 23 | | | | | | | | |
| set2_695 | IGHV3- | IGHJ6 | 6 | 43 | 171 | 253 | 343 | 1113 | 1196 |
| | 7 | | | | | | | | |
| set2_696 | IGHV3- | IGHJ6 | 12 | 38 | 146 | 254 | 304 | 1114 | 1196 |
| | 23 | | | | | | | | |
| set2_697 | IGHV5- | IGHJ5 | 17 | 61 | 157 | 229 | 326 | 1115 | 1197 |
| | 51 | | | | | | | | |
| set2_698 | IGHV3- | IGHJ6 | 2 | 39 | 141 | 301 | 409 | 1116 | 1196 |
| | 30 | | | | | | | | |
| set2_699 | IGHV4- | IGHJ3 | 11 | 88 | 145 | 225 | 410 | 1117 | 1198 |
| | 39 | | | | | | | | |
| set2_700 | IGHV3- | IGHJ6 | 6 | 43 | 171 | 253 | 343 | 1118 | 1196 |
| | 7 | | | | | | | | |
| set2_701 | IGHV4- | IGHJ4 | 15 | 66 | 161 | 242 | 312 | 1119 | 1197 |
| | 34 | | | | | | | | |
| set2_702 | IGHV1- | IGHJ6 | 4 | 90 | 199 | 288 | 396 | 1120 | 1196 |
| | 69 | | | | | | | | |
| set2_703 | IGHV4- | IGHJ5 | 11 | 88 | 145 | 225 | 355 | 1121 | 1197 |
| | 39 | | | | | | | | |
| set2_704 | IGHV3- | IGHJ6 | 6 | 43 | 171 | 253 | 343 | 1122 | 1196 |
| | 7 | | | | | | | | |
| set2_705 | IGHV4- | IGHJ6 | 9 | 47 | 143 | 225 | 312 | 1123 | 1196 |
| | 59 | | | | | | | | |
| set2_706 | IGHV3- | IGHJ6 | 20 | 65 | 186 | 272 | 341 | 1124 | 1196 |
| | 11 | | | | | | | | |
| set2_707 | IGHV4- | IGHJ4 | 15 | 66 | 161 | 242 | 312 | 1125 | 1197 |
| | 34 | | | | | | | | |
| set2_708 | IGHV1- | IGHJ6 | 4 | 90 | 199 | 288 | 396 | 1126 | 1200 |
| | 69 | | | | | | | | |
| set2_709 | IGHV1- | IGHJ6 | 4 | 90 | 199 | 288 | 396 | 1127 | 1196 |
| | 69 | | | | | | | | |
| set2_710 | IGHV3- | IGHJ3 | 6 | 129 | 212 | 302 | 411 | 1128 | 1198 |
| | 7 | | | | | | | | |
| set2_711 | IGHV4- | IGHJ6 | 11 | 88 | 145 | 225 | 355 | 1129 | 1196 |
| | 39 | | | | | | | | |
| set2_712 | IGHV4- | IGHJ6 | 15 | 66 | 161 | 242 | 312 | 1130 | 1196 |
| | 34 | | | | | | | | |
| set2_713 | IGHV1- | IGHJ3 | 14 | 84 | 148 | 230 | 351 | 1131 | 1198 |
| | 2 | | | | | | | | |
| set2_714 | IGHV3- | IGHJ4 | 2 | 39 | 135 | 216 | 304 | 1132 | 1197 |
| | 30 | | | | | | | | |
| set2_715 | IGHV4- | IGHJ6 | 8 | 109 | 194 | 225 | 355 | 1133 | 1196 |
| | 31 | | | | | | | | |
| set2_716 | IGHV4- | IGHJ6 | 11 | 88 | 145 | 225 | 355 | 1134 | 1196 |
| | 39 | | | | | | | | |
| set2_717 | IGHV3- | IGHJ6 | 7 | 58 | 169 | 251 | 341 | 1135 | 1200 |
| | 21 | | | | | | | | |
| set2_718 | IGHV3- | IGHJ3 | 12 | 130 | 213 | 303 | 412 | 1136 | 1196 |
| | 23 | | | | | | | | |
| set2_719 | IGHV3- | IGHJ6 | 6 | 106 | 190 | 280 | 378 | 1137 | 1200 |
| | 13 | | | | | | | | |
| set2_720 | IGHV4- | IGHJ3 | 9 | 131 | 143 | 225 | 312 | 1138 | 1198 |
| | 61 | | | | | | | | |
| set2_721 | IGHV3- | IGHJ4 | 7 | 44 | 140 | 222 | 309 | 1139 | 1197 |
| | 15 | | | | | | | | |
| set2_722 | IGHV2- | IGHJ6 | 33 | 74 | 192 | 249 | 339 | 1140 | 1196 |
| | 70 | | | | | | | | |
| set2_723 | IGHV4- | IGHJ6 | 15 | 66 | 161 | 242 | 312 | 1141 | 1196 |
| | 34 | | | | | | | | |
| set2_724 | IGHV3- | IGHJ6 | 20 | 65 | 186 | 272 | 341 | 1142 | 1196 |
| | 11 | | | | | | | | |
| set2_725 | IGHV4- | IGHJ6 | 9 | 47 | 143 | 225 | 312 | 1143 | 1196 |
| | 59 | | | | | | | | |
| set2_726 | IGHV3- | IGHJ4 | 2 | 39 | 135 | 218 | 304 | 1144 | 1197 |
| | 33 | | | | | | | | |
| set2_727 | IGHV1- | IGHJ6 | 30 | 98 | 184 | 267 | 364 | 1145 | 1196 |
| | 24 | | | | | | | | |
| set2_728 | IGHV3- | IGHJ5 | 20 | 65 | 186 | 272 | 341 | 1146 | 1197 |
| | 11 | | | | | | | | |
| set2_729 | IGHV3- | IGHJ2 | 6 | 106 | 190 | 277 | 378 | 1147 | 1203 |
| | 13 | | | | | | | | |
| set2_730 | IGHV4- | IGHJ6 | 15 | 66 | 161 | 242 | 312 | 1148 | 1196 |
| | 34 | | | | | | | | |
| set2_731 | IGHV1- | IGHJ6 | 14 | 70 | 164 | 245 | 334 | 1149 | 1196 |
| | 18 | | | | | | | | |
| set2_732 | IGHV3- | IGHJ6 | 2 | 39 | 181 | 216 | 304 | 1150 | 1196 |
| | 30 | | | | | | | | |
| set2_733 | IGHV4- | IGHJ6 | 15 | 66 | 161 | 242 | 312 | 1151 | 1196 |
| | 34 | | | | | | | | |
| set2_734 | IGHV3- | IGHJ6 | 6 | 43 | 171 | 253 | 343 | 1152 | 1196 |
| | 7 | | | | | | | | |
| set2_735 | IGHV4- | IGHJ5 | 11 | 88 | 145 | 225 | 355 | 1153 | 1197 |
| | 39 | | | | | | | | |
| set2_736 | IGHV1- | IGHJ4 | 14 | 70 | 164 | 245 | 334 | 1154 | 1197 |
| | 18 | | | | | | | | |
| set2_737 | IGHV3- | IGHJ3 | 6 | 132 | 214 | 221 | 413 | 1155 | 1198 |
| | 74 | | | | | | | | |
| set2_738 | IGHV4- | IGHJ5 | 9 | 47 | 143 | 225 | 312 | 1156 | 1197 |
| | 59 | | | | | | | | |
| set2_739 | IGHV4- | IGHJ6 | 11 | 88 | 145 | 225 | 355 | 1157 | 1196 |
| | 39 | | | | | | | | |
| set2_740 | IGHV3- | IGHJ4 | 2 | 39 | 135 | 216 | 304 | 1158 | 1197 |
| | 30 | | | | | | | | |
| set2_741 | IGHV1- | IGHJ6 | 14 | 70 | 164 | 245 | 334 | 1159 | 1196 |
| | 18 | | | | | | | | |
| set2_742 | IGHV3- | IGHJ6 | 20 | 65 | 186 | 272 | 341 | 1160 | 1196 |
| | 11 | | | | | | | | |
| set2_743 | IGHV3- | IGHJ6 | 6 | 43 | 171 | 253 | 343 | 1161 | 1196 |
| | 7 | | | | | | | | |
| set2_744 | IGHV3- | IGHJ6 | 6 | 43 | 171 | 253 | 343 | 1162 | 1196 |
| | 7 | | | | | | | | |
| set2_745 | IGHV1- | IGHJ6 | 14 | 70 | 164 | 245 | 334 | 1163 | 1196 |
| | 18 | | | | | | | | |
| set2_746 | IGHV1- | IGHJ6 | 14 | 70 | 164 | 245 | 334 | 1164 | 1196 |
| | 18 | | | | | | | | |
| set2_747 | IGHV1- | IGHJ6 | 30 | 98 | 184 | 267 | 364 | 1165 | 1196 |
| | 24 | | | | | | | | |
| set2_748 | IGHV4- | IGHJ6 | 15 | 66 | 161 | 242 | 312 | 1166 | 1196 |
| | 34 | | | | | | | | |
| set2_749 | IGHV1- | IGHJ6 | 14 | 70 | 164 | 245 | 334 | 1167 | 1196 |
| | 18 | | | | | | | | |
| set2_750 | IGHV1- | IGHJ6 | 4 | 90 | 199 | 288 | 396 | 1168 | 1196 |
| | 69 | | | | | | | | |
| set2_751 | IGHV3- | IGHJ6 | 6 | 43 | 171 | 253 | 343 | 1169 | 1196 |
| | 7 | | | | | | | | |
| set2_752 | IGHV1- | IGHJ6 | 4 | 90 | 199 | 288 | 396 | 1170 | 1196 |
| | 69 | | | | | | | | |
| set2_753 | IGHV3- | IGHJ4 | 2 | 133 | 135 | 216 | 414 | 1171 | 1197 |
| | 30 | | | | | | | | |
| set2_754 | IGHV3- | IGHJ3 | 7 | 58 | 169 | 251 | 341 | 1172 | 1198 |
| | 21 | | | | | | | | |
| set2_755 | IGHV3- | IGHJ6 | 2 | 39 | 135 | 218 | 304 | 1173 | 1196 |
| | 33 | | | | | | | | |
| set2_756 | IGHV3- | IGHJ6 | 6 | 43 | 171 | 253 | 343 | 1174 | 1196 |
| | 7 | | | | | | | | |
| set2_757 | IGHV1- | IGHJ6 | 14 | 70 | 164 | 245 | 334 | 1175 | 1196 |
| | 18 | | | | | | | | |
| set2_758 | IGHV3- | IGHJ4 | 6 | 43 | 171 | 253 | 343 | 1176 | 1197 |
| | 7 | | | | | | | | |
| set2_759 | IGHV1- | IGHJ6 | 14 | 84 | 148 | 230 | 351 | 1177 | 1196 |
| | 2 | | | | | | | | |
| set2_760 | IGHV3- | IGHJ6 | 7 | 58 | 169 | 251 | 341 | 1178 | 1196 |
| | 21 | | | | | | | | |
| set2_761 | IGHV4- | IGHJ6 | 15 | 66 | 161 | 242 | 312 | 1179 | 1196 |
| | 34 | | | | | | | | |
| set2_762 | IGHV1- | IGHJ6 | 14 | 70 | 164 | 245 | 334 | 1180 | 1196 |
| | 18 | | | | | | | | |
| set2_763 | IGHV3- | IGHJ6 | 20 | 65 | 186 | 272 | 341 | 1181 | 1196 |
| | 11 | | | | | | | | |
| set2_764 | IGHV4- | IGHJ6 | 9 | 47 | 175 | 256 | 347 | 1182 | 1196 |
| | 4 | | | | | | | | |
| set2_765 | IGHV4- | IGHJ6 | 15 | 66 | 161 | 242 | 312 | 1183 | 1196 |
| | 34 | | | | | | | | |
| set2_766 | IGHV1- | IGHJ6 | 14 | 70 | 164 | 245 | 334 | 1184 | 1196 |
| | 18 | | | | | | | | |
| set2_767 | IGHV1- | IGHJ6 | 14 | 70 | 164 | 245 | 334 | 1185 | 1196 |
| | 18 | | | | | | | | |
| set2_768 | IGHV4- | IGHJ6 | 15 | 134 | 161 | 242 | 312 | 1186 | 1196 |
| | 34 | | | | | | | | |
| set2_769 | IGHV1- | IGHJ6 | 14 | 70 | 164 | 245 | 334 | 1187 | 1196 |
| | 18 | | | | | | | | |
| set2_770 | IGHV1- | IGHJ6 | 14 | 70 | 215 | 245 | 415 | 1188 | 1196 |
| | 18 | | | | | | | | |
| set2_771 | IGHV3- | IGHJ6 | 2 | 39 | 135 | 218 | 304 | 1189 | 1196 |
| | 33 | | | | | | | | |
| set2_772 | IGHV3- | IGHJ6 | 2 | 38 | 135 | 216 | 304 | 1190 | 1200 |
| | 30 | | | | | | | | |
| set2_773 | IGHV3- | IGHJ6 | 2 | 39 | 135 | 218 | 304 | 1191 | 1196 |
| | 33 | | | | | | | | |
| set2_774 | IGHV3- | IGHJ6 | 2 | 38 | 135 | 216 | 304 | 1192 | 1196 |
| | 30 | | | | | | | | |
| set2_775 | IGHV3- | IGHJ6 | 2 | 39 | 135 | 218 | 304 | 1193 | 1196 |
| | 33 | | | | | | | | |
| set2_776 | IGHV3- | IGHJ6 | 2 | 39 | 135 | 218 | 304 | 1194 | 1196 |
| | 33 | | | | | | | | |
| set2_777 | IGHV3- | IGHJ6 | 2 | 39 | 135 | 218 | 304 | 1195 | 1196 |
| | 33 | | | | | | | | |

TABLE 2
Polypeptide sequences of immunoglobulin
heavy chain variable domains (VHs)

| | Polypeptide sequence |
|---|---|
| VH name | (SEQ ID NO) |
| set1_1 | 1 |
| set1_2 | 1219 |
| set1_3 | 1220 |
| set1_4 | 1221 |
| set1_5 | 1222 |
| set2_1 | 1223 |
| set2_2 | 1224 |
| set2_3 | 1225 |
| set2_4 | 1226 |
| set2_5 | 1227 |
| set2_6 | 1228 |
| set2_7 | 1229 |
| set2_8 | 1230 |
| set2_9 | 1231 |
| set2_10 | 1232 |
| set2_11 | 1233 |
| set2_12 | 1234 |
| set2_13 | 1235 |
| set2_14 | 1236 |
| set2_15 | 1237 |
| set2_16 | 1238 |
| set2_17 | 1239 |
| set2_18 | 1240 |
| set2_19 | 1241 |
| set2_20 | 1242 |
| set2_21 | 1243 |
| set2_22 | 1244 |
| set2_23 | 1245 |
| set2_24 | 1246 |
| set2_25 | 1247 |
| set2_26 | 1248 |
| set2_27 | 1249 |
| set2_28 | 1250 |
| set2_29 | 1251 |
| set2_30 | 1252 |
| set2_31 | 1253 |
| set2_32 | 1254 |
| set2_33 | 1255 |
| set2_34 | 1256 |
| set2_35 | 1257 |
| set2_36 | 1258 |
| set2_37 | 1259 |
| set2_38 | 1260 |
| set2_39 | 1261 |
| set2_40 | 1262 |
| set2_41 | 1263 |
| set2_42 | 1264 |
| set2_43 | 1265 |
| set2_44 | 1266 |
| set2_45 | 1267 |
| set2_46 | 1268 |
| set2_47 | 1269 |
| set2_48 | 1270 |
| set2_49 | 1271 |
| set2_50 | 1272 |
| set2_51 | 1273 |
| set2_52 | 1274 |
| set2_53 | 1275 |
| set2_54 | 1276 |
| set2_55 | 1277 |
| set2_56 | 1278 |
| set2_57 | 1279 |
| set2_58 | 1280 |
| set2_59 | 1281 |
| set2_60 | 1282 |
| set2_61 | 1283 |
| set2_62 | 1284 |
| set2_63 | 1285 |
| set2_64 | 1286 |
| set2_65 | 1287 |
| set2_66 | 1288 |
| set2_67 | 1289 |
| set2_68 | 1290 |
| set2_69 | 1291 |
| set2_70 | 1292 |
| set2_71 | 1293 |
| set2_72 | 1294 |
| set2_73 | 1295 |
| set2_74 | 1296 |
| set2_75 | 1297 |
| set2_76 | 1298 |
| set2_77 | 1299 |
| set2_78 | 1300 |
| set2_79 | 1301 |
| set2_80 | 1302 |
| set2_81 | 1303 |
| set2_82 | 1304 |
| set2_83 | 1305 |
| set2_84 | 1306 |
| set2_85 | 1307 |
| set2_86 | 1308 |
| set2_87 | 1309 |
| set2_88 | 1310 |
| set2_89 | 1311 |
| set2_90 | 1312 |
| set2_91 | 1313 |
| set2_92 | 1314 |
| set2_93 | 1315 |
| set2_94 | 1316 |
| set2_95 | 1317 |
| set2_96 | 1318 |
| set2_97 | 1319 |
| set2_98 | 1320 |
| set2_99 | 1321 |
| set2_100 | 1322 |
| set2_101 | 1323 |
| set2_102 | 1324 |
| set2_103 | 1325 |
| set2_104 | 1326 |
| set2_105 | 1327 |
| set2_106 | 1328 |
| set2_107 | 1329 |
| set2_108 | 1330 |
| set2_109 | 1331 |
| set2_110 | 1332 |
| set2_111 | 1333 |
| set2_112 | 1334 |
| set2_113 | 1335 |
| set2_114 | 1336 |
| set2_115 | 1337 |
| set2_116 | 1338 |
| set2_117 | 1339 |
| set2_118 | 1340 |
| set2_119 | 1341 |
| set2_120 | 1342 |
| set2_121 | 1343 |
| set2_122 | 1344 |
| set2_123 | 1345 |
| set2_124 | 1346 |
| set2_125 | 1347 |
| set2_126 | 1348 |
| set2_127 | 1349 |
| set2_128 | 1350 |
| set2_129 | 1351 |
| set2_130 | 1352 |
| set2_131 | 1353 |
| set2_132 | 1354 |
| set2_133 | 1355 |
| set2_134 | 1356 |
| set2_135 | 1357 |
| set2_136 | 1358 |
| set2_137 | 1359 |
| set2_138 | 1360 |
| set2_139 | 1361 |
| set2_140 | 1362 |
| set2_141 | 1363 |
| set2_142 | 1364 |
| set2_143 | 1365 |
| set2_144 | 1366 |
| set2_145 | 1367 |
| set2_146 | 1368 |
| set2_147 | 1369 |
| set2_148 | 1370 |
| set2_149 | 1371 |
| set2_150 | 1372 |
| set2_151 | 1373 |
| set2_152 | 1374 |
| set2_153 | 1375 |
| set2_154 | 1376 |
| set2_155 | 1377 |
| set2_156 | 1378 |
| set2_157 | 1379 |
| set2_158 | 1380 |
| set2_159 | 1381 |
| set2_160 | 1382 |
| set2_161 | 1383 |
| set2_162 | 1384 |
| set2_163 | 1385 |
| set2_164 | 1386 |
| set2_165 | 1387 |
| set2_166 | 1388 |
| set2_167 | 1389 |
| set2_168 | 1390 |
| set2_169 | 1391 |
| set2_170 | 1392 |
| set2_171 | 1393 |
| set2_172 | 1394 |
| set2_173 | 1395 |
| set2_174 | 1396 |
| set2_175 | 1397 |
| set2_176 | 1398 |
| set2_177 | 1399 |
| set2_178 | 1400 |
| set2_179 | 1401 |
| set2_180 | 1402 |
| set2_181 | 1403 |
| set2_182 | 1404 |
| set2_183 | 1405 |
| set2_184 | 1406 |
| set2_185 | 1407 |
| set2_186 | 1408 |
| set2_187 | 1409 |
| set2_188 | 1410 |
| set2_189 | 1411 |
| set2_190 | 1412 |
| set2_191 | 1413 |
| set2_192 | 1414 |
| set2_193 | 1415 |
| set2_194 | 1416 |
| set2_195 | 1417 |
| set2_196 | 1418 |
| set2_197 | 1419 |
| set2_198 | 1420 |
| set2_199 | 1421 |
| set2_200 | 1422 |
| set2_201 | 1423 |
| set2_202 | 1424 |
| set2_203 | 1425 |
| set2_204 | 1426 |
| set2_205 | 1427 |
| set2_206 | 1428 |
| set2_207 | 1429 |
| set2_208 | 1430 |
| set2_209 | 1431 |
| set2_210 | 1432 |
| set2_211 | 1433 |
| set2_212 | 1434 |
| set2_213 | 1435 |
| set2_214 | 1436 |
| set2_215 | 1437 |
| set2_216 | 1438 |
| set2_217 | 1439 |
| set2_218 | 1440 |
| set2_219 | 1441 |
| set2_220 | 1442 |
| set2_221 | 1443 |
| set2_222 | 1444 |
| set2_223 | 1445 |
| set2_224 | 1446 |
| set2_225 | 1447 |
| set2_226 | 1448 |
| set2_227 | 1449 |
| set2_228 | 1450 |
| set2_229 | 1451 |
| set2_230 | 1452 |
| set2_231 | 1453 |
| set2_232 | 1454 |
| set2_233 | 1455 |
| set2_234 | 1456 |
| set2_235 | 1457 |
| set2_236 | 1458 |
| set2_237 | 1459 |
| set2_238 | 1460 |
| set2_239 | 1461 |
| set2_240 | 1462 |
| set2_241 | 1463 |
| set2_242 | 1464 |
| set2_243 | 1465 |
| set2_244 | 1466 |
| set2_245 | 1467 |
| set2_246 | 1468 |
| set2_247 | 1469 |
| set2_248 | 1470 |
| set2_249 | 1471 |
| set2_250 | 1472 |
| set2_251 | 1473 |
| set2_252 | 1474 |
| set2_253 | 1475 |
| set2_254 | 1476 |
| set2_255 | 1477 |
| set2_256 | 1478 |
| set2_257 | 1479 |
| set2_258 | 1480 |
| set2_259 | 1481 |
| set2_260 | 1482 |
| set2_261 | 1483 |
| set2_262 | 1484 |
| set2_263 | 1485 |
| set2_264 | 1486 |
| set2_265 | 1487 |
| set2_266 | 1488 |
| set2_267 | 1489 |
| set2_268 | 1490 |
| set2_269 | 1491 |
| set2_270 | 1492 |
| set2_271 | 1493 |
| set2_272 | 1474 |
| set2_273 | 1494 |
| set2_274 | 1495 |
| set2_275 | 1496 |
| set2_276 | 1497 |
| set2_277 | 1498 |
| set2_278 | 1499 |
| set2_279 | 1500 |
| set2_280 | 1501 |
| set2_281 | 1502 |
| set2_282 | 1503 |
| set2_283 | 1504 |
| set2_284 | 1505 |
| set2_285 | 1506 |
| set2_286 | 1507 |
| set2_287 | 1508 |
| set2_288 | 1509 |
| set2_289 | 1510 |
| set2_290 | 1511 |
| set2_291 | 1512 |
| set2_292 | 1513 |
| set2_293 | 1514 |
| set2_294 | 1515 |
| set2_295 | 1516 |
| set2_296 | 1517 |
| set2_297 | 1518 |
| set2_298 | 1519 |
| set2_299 | 1520 |
| set2_300 | 1521 |
| set2_301 | 1522 |
| set2_302 | 1523 |
| set2_303 | 1524 |
| set2_304 | 1525 |
| set2_305 | 1526 |
| set2_306 | 1527 |
| set2_307 | 1528 |
| set2_308 | 1529 |
| set2_309 | 1530 |
| set2_310 | 1531 |
| set2_311 | 1532 |
| set2_312 | 1533 |
| set2_313 | 1534 |
| set2_314 | 1535 |
| set2_315 | 1536 |
| set2_316 | 1537 |
| set2_317 | 1538 |
| set2_318 | 1539 |
| set2_319 | 1540 |
| set2_320 | 1541 |
| set2_321 | 1542 |
| set2_322 | 1543 |
| set2_323 | 1544 |
| set2_324 | 1545 |
| set2_325 | 1546 |
| set2_326 | 1547 |
| set2_327 | 1548 |
| set2_328 | 1549 |
| set2_329 | 1550 |
| set2_330 | 1551 |
| set2_331 | 1552 |
| set2_332 | 1553 |
| set2_333 | 1554 |
| set2_334 | 1555 |
| set2_335 | 1556 |
| set2_336 | 1557 |
| set2_337 | 1558 |
| set2_338 | 1559 |
| set2_339 | 1560 |
| set2_340 | 1561 |
| set2_341 | 1562 |
| set2_342 | 1563 |
| set2_343 | 1564 |
| set2_344 | 1565 |
| set2_345 | 1566 |
| set2_346 | 1567 |
| set2_347 | 1568 |
| set2_348 | 1569 |
| set2_349 | 1570 |
| set2_350 | 1571 |
| set2_351 | 1572 |
| set2_352 | 1573 |
| set2_353 | 1574 |
| set2_354 | 1575 |
| set2_355 | 1576 |
| set2_356 | 1577 |
| set2_357 | 1578 |
| set2_358 | 1579 |
| set2_359 | 1580 |
| set2_360 | 1581 |
| set2_361 | 1582 |
| set2_362 | 1583 |
| set2_363 | 1584 |
| set2_364 | 1585 |
| set2_365 | 1586 |
| set2_366 | 1587 |
| set2_367 | 1588 |
| set2_368 | 1589 |
| set2_369 | 1590 |
| set2_370 | 1591 |
| set2_371 | 1592 |
| set2_372 | 1593 |
| set2_373 | 1594 |
| set2_374 | 1595 |
| set2_375 | 1596 |
| set2_376 | 1597 |
| set2_377 | 1598 |
| set2_378 | 1599 |
| set2_379 | 1600 |
| set2_380 | 1601 |
| set2_381 | 1602 |
| set2_382 | 1603 |
| set2_383 | 1604 |
| set2_384 | 1605 |
| set2_385 | 1606 |
| set2_386 | 1607 |
| set2_387 | 1608 |
| set2_388 | 1609 |
| set2_389 | 1610 |
| set2_390 | 1611 |
| set2_391 | 1612 |
| set2_392 | 1613 |
| set2_393 | 1614 |
| set2_394 | 1615 |
| set2_395 | 1616 |
| set2_396 | 1617 |
| set2_397 | 1618 |
| set2_398 | 1619 |
| set2_399 | 1620 |
| set2_400 | 1621 |
| set2_401 | 1622 |
| set2_402 | 1623 |
| set2_403 | 1624 |
| set2_404 | 1625 |
| set2_405 | 1626 |
| set2_406 | 1627 |
| set2_407 | 1628 |
| set2_408 | 1629 |
| set2_409 | 1630 |
| set2_410 | 1631 |
| set2_411 | 1632 |
| set2_412 | 1633 |
| set2_413 | 1634 |
| set2_414 | 1635 |
| set2_415 | 1636 |
| set2_416 | 1637 |
| set2_417 | 1638 |
| set2_418 | 1639 |
| set2_419 | 1640 |
| set2_420 | 1641 |
| set2_421 | 1642 |
| set2_422 | 1643 |
| set2_423 | 1644 |
| set2_424 | 1645 |
| set2_425 | 1646 |
| set2_426 | 1647 |
| set2_427 | 1648 |
| set2_428 | 1649 |
| set2_429 | 1650 |
| set2_430 | 1651 |
| set2_431 | 1652 |
| set2_432 | 1653 |
| set2_433 | 1654 |
| set2_434 | 1655 |
| set2_435 | 1656 |
| set2_436 | 1657 |
| set2_437 | 1658 |
| set2_438 | 1659 |
| set2_439 | 1660 |
| set2_440 | 1661 |
| set2_441 | 1662 |
| set2_442 | 1663 |
| set2_443 | 1664 |
| set2_444 | 1665 |
| set2_445 | 1666 |
| set2_446 | 1667 |
| set2_447 | 1668 |
| set2_448 | 1669 |
| set2_449 | 1670 |
| set2_450 | 1671 |
| set2_451 | 1672 |
| set2_452 | 1673 |
| set2_453 | 1674 |
| set2_454 | 1675 |
| set2_455 | 1676 |
| set2_456 | 1677 |
| set2_457 | 1678 |
| set2_458 | 1679 |
| set2_459 | 1680 |
| set2_460 | 1681 |
| set2_461 | 1682 |
| set2_462 | 1683 |
| set2_463 | 1684 |
| set2_464 | 1685 |
| set2_465 | 1686 |
| set2_466 | 1687 |
| set2_467 | 1688 |
| set2_468 | 1689 |
| set2_469 | 1690 |
| set2_470 | 1691 |
| set2_471 | 1692 |
| set2_472 | 1693 |
| set2_473 | 1694 |
| set2_474 | 1695 |
| set2_475 | 1696 |
| set2_476 | 1697 |
| set2_477 | 1698 |
| set2_478 | 1699 |
| set2_479 | 1700 |
| set2_480 | 1701 |
| set2_481 | 1702 |
| set2_482 | 1703 |
| set2_483 | 1704 |
| set2_484 | 1705 |
| set2_485 | 1706 |
| set2_486 | 1707 |
| set2_487 | 1708 |
| set2_488 | 1709 |
| set2_489 | 1710 |
| set2_490 | 1711 |
| set2_491 | 1712 |
| set2_492 | 1713 |
| set2_493 | 1714 |
| set2_494 | 1715 |
| set2_495 | 1716 |
| set2_496 | 1717 |
| set2_497 | 1718 |
| set2_498 | 1719 |
| set2_499 | 1720 |
| set2_500 | 1721 |
| set2_501 | 1722 |
| set2_502 | 1723 |
| set2_503 | 1724 |
| set2_504 | 1725 |
| set2_505 | 1726 |
| set2_506 | 1727 |
| set2_507 | 1728 |
| set2_508 | 1729 |
| set2_509 | 1730 |
| set2_510 | 1731 |
| set2_511 | 1732 |
| set2_512 | 1733 |
| set2_513 | 1734 |
| set2_514 | 1735 |
| set2_515 | 1736 |
| set2_516 | 1737 |
| set2_517 | 1738 |
| set2_518 | 1739 |
| set2_519 | 1740 |
| set2_520 | 1741 |
| set2_521 | 1742 |
| set2_522 | 1743 |
| set2_523 | 1744 |
| set2_524 | 1745 |
| set2_525 | 1746 |
| set2_526 | 1747 |
| set2_527 | 1748 |
| set2_528 | 1749 |
| set2_529 | 1750 |
| set2_530 | 1751 |
| set2_531 | 1752 |
| set2_532 | 1753 |
| set2_533 | 1754 |
| set2_534 | 1755 |
| set2_535 | 1756 |
| set2_536 | 1757 |
| set2_537 | 1758 |
| set2_538 | 1759 |
| set2_539 | 1760 |
| set2_540 | 1761 |
| set2_541 | 1762 |
| set2_542 | 1763 |
| set2_543 | 1764 |
| set2_544 | 1765 |
| set2_545 | 1766 |
| set2_546 | 1767 |
| set2_547 | 1768 |
| set2_548 | 1769 |
| set2_549 | 1770 |
| set2_550 | 1771 |
| set2_551 | 1772 |
| set2_552 | 1773 |
| set2_553 | 1774 |
| set2_554 | 1775 |
| set2_555 | 1776 |
| set2_556 | 1777 |
| set2_557 | 1778 |
| set2_558 | 1779 |
| set2_559 | 1780 |
| set2_560 | 1781 |
| set2_561 | 1782 |
| set2_562 | 1783 |
| set2_563 | 1784 |
| set2_564 | 1785 |
| set2_565 | 1786 |
| set2_566 | 1787 |
| set2_567 | 1788 |
| set2_568 | 1789 |
| set2_569 | 1790 |
| set2_570 | 1791 |
| set2_571 | 1792 |
| set2_572 | 1793 |
| set2_573 | 1794 |
| set2_574 | 1795 |
| set2_575 | 1796 |
| set2_576 | 1797 |
| set2_577 | 1798 |
| set2_578 | 1799 |
| set2_579 | 1800 |
| set2_580 | 1801 |
| set2_581 | 1802 |
| set2_582 | 1803 |
| set2_583 | 1804 |
| set2_584 | 1805 |
| set2_585 | 1806 |
| set2_586 | 1807 |
| set2_587 | 1808 |
| set2_588 | 1809 |
| set2_589 | 1810 |
| set2_590 | 1811 |
| set2_591 | 1812 |
| set2_592 | 1813 |
| set2_593 | 1814 |
| set2_594 | 1815 |
| set2_595 | 1816 |
| set2_596 | 1817 |
| set2_597 | 1818 |
| set2_598 | 1819 |
| set2_599 | 1820 |
| set2_600 | 1821 |
| set2_601 | 1822 |
| set2_602 | 1823 |
| set2_603 | 1824 |
| set2_604 | 1825 |
| set2_605 | 1826 |
| set2_606 | 1827 |
| set2_607 | 1828 |
| set2_608 | 1829 |
| set2_609 | 1830 |
| set2_610 | 1831 |
| set2_611 | 1832 |
| set2_612 | 1833 |
| set2_613 | 1834 |
| set2_614 | 1835 |
| set2_615 | 1836 |
| set2_616 | 1837 |
| set2_617 | 1838 |
| set2_618 | 1839 |
| set2_619 | 1840 |
| set2_620 | 1841 |
| set2_621 | 1842 |
| set2_622 | 1843 |
| set2_623 | 1844 |
| set2_624 | 1845 |
| set2_625 | 1846 |
| set2_626 | 1847 |
| set2_627 | 1848 |
| set2_628 | 1849 |
| set2_629 | 1850 |
| set2_630 | 1851 |
| set2_631 | 1852 |
| set2_632 | 1853 |
| set2_633 | 1854 |
| set2_634 | 1855 |
| set2_635 | 1856 |
| set2_636 | 1857 |
| set2_637 | 1858 |
| set2_638 | 1859 |
| set2_639 | 1860 |
| set2_640 | 1861 |
| set2_641 | 1862 |
| set2_642 | 1863 |
| set2_643 | 1864 |
| set2_644 | 1865 |
| set2_645 | 1866 |
| set2_646 | 1867 |
| set2_647 | 1868 |
| set2_648 | 1869 |
| set2_649 | 1870 |
| set2_650 | 1871 |
| set2_651 | 1872 |
| set2_652 | 1873 |
| set2_653 | 1874 |
| set2_654 | 1875 |
| set2_655 | 1876 |
| set2_656 | 1877 |
| set2_657 | 1878 |
| set2_658 | 1879 |
| set2_659 | 1880 |
| set2_660 | 1881 |
| set2_661 | 1882 |
| set2_662 | 1883 |
| set2_663 | 1884 |
| set2_664 | 1885 |
| set2_665 | 1886 |
| set2_666 | 1887 |
| set2_667 | 1888 |
| set2_668 | 1889 |
| set2_669 | 1890 |
| set2_670 | 1891 |
| set2_671 | 1892 |
| set2_672 | 1893 |
| set2_673 | 1894 |
| set2_674 | 1895 |
| set2_675 | 1896 |
| set2_676 | 1897 |
| set2_677 | 1898 |
| set2_678 | 1899 |
| set2_679 | 1900 |
| set2_680 | 1901 |
| set2_681 | 1902 |
| set2_682 | 1903 |
| set2_683 | 1904 |
| set2_684 | 1905 |
| set2_685 | 1906 |
| set2_686 | 1907 |
| set2_687 | 1908 |
| set2_688 | 1909 |
| set2_689 | 1910 |
| set2_690 | 1911 |
| set2_691 | 1912 |
| set2_692 | 1913 |
| set2_693 | 1914 |
| set2_694 | 1915 |
| set2_695 | 1916 |
| set2_696 | 1917 |
| set2_697 | 1918 |
| set2_698 | 1919 |
| set2_699 | 1920 |
| set2_700 | 1921 |
| set2_701 | 1922 |
| set2_702 | 1923 |
| set2_703 | 1924 |
| set2_704 | 1925 |
| set2_705 | 1926 |
| set2_706 | 1927 |
| set2_707 | 1928 |
| set2_708 | 1929 |
| set2_709 | 1930 |
| set2_710 | 1931 |
| set2_711 | 1932 |
| set2_712 | 1933 |
| set2_713 | 1934 |
| set2_714 | 1935 |
| set2_715 | 1936 |
| set2_716 | 1937 |
| set2_717 | 1938 |
| set2_718 | 1939 |
| set2_719 | 1940 |
| set2_720 | 1941 |
| set2_721 | 1942 |
| set2_722 | 1943 |
| set2_723 | 1944 |
| set2_724 | 1945 |
| set2_725 | 1946 |
| set2_726 | 1947 |
| set2_727 | 1948 |
| set2_728 | 1949 |
| set2_729 | 1950 |
| set2_730 | 1951 |
| set2_731 | 1952 |
| set2_732 | 1953 |
| set2_733 | 1954 |
| set2_734 | 1955 |
| set2_735 | 1956 |
| set2_736 | 1957 |
| set2_737 | 1958 |
| set2_738 | 1959 |
| set2_739 | 1960 |
| set2_740 | 1961 |
| set2_741 | 1962 |
| set2_742 | 1963 |
| set2_743 | 1964 |
| set2_744 | 1965 |
| set2_745 | 1966 |
| set2_746 | 1967 |
| set2_747 | 1968 |
| set2_748 | 1969 |
| set2_749 | 1970 |
| set2_750 | 1971 |
| set2_751 | 1972 |
| set2_752 | 1973 |
| set2_753 | 1974 |
| set2_754 | 1975 |
| set2_755 | 1976 |
| set2_756 | 1977 |
| set2_757 | 1978 |
| set2_758 | 1979 |
| set2_759 | 1980 |
| set2_760 | 1981 |
| set2_761 | 1982 |
| set2_762 | 1983 |
| set2_763 | 1984 |
| set2_764 | 1985 |
| set2_765 | 1986 |
| set2_766 | 1987 |
| set2_767 | 1988 |
| set2_768 | 1989 |
| set2_769 | 1990 |
| set2_770 | 1991 |
| set2_771 | 1992 |
| set2_772 | 1993 |
| set2_773 | 1994 |
| set2_774 | 1995 |
| set2_775 | 1996 |
| set2_776 | 1997 |
| set2_777 | 1998 |

In one embodiment there is provided a polypeptide comprising:

a sequence (such as a CDRH1 sequence) comprising or consisting of a sequence sharing 80% or greater sequence identity with a CDRH1 sequence as shown in Table 1 and/or

a sequence (such as a CDRH2 sequence) comprising or consisting of a sequence sharing 80% or greater sequence identity with a CDRH2 sequence as shown in Table 1 and/or

a sequence (such as a CDRH3 sequence) comprising or consisting of a sequence sharing 80% or greater sequence identity with a CDRH3 sequence as shown in Table 1.

Suitably the polypeptide comprises

a sequence (such as a CDRH1 sequence) comprising or consisting of a sequence sharing 90% or greater sequence identity with a CDRH1 sequence as shown in Table 1 and/or

a sequence (such as a CDRH2 sequence) comprising or consisting of a sequence sharing 90% or greater sequence identity with a CDRH2 sequence as shown in Table 1 and/or

a sequence (such as a CDRH3 sequence) comprising or consisting of a sequence sharing 90% or greater sequence identity with a CDRH3 sequence as shown in Table 1.

More suitably the polypeptide comprises

a sequence (such as a CDRH1 sequence) comprising or consisting of a CDRH1 sequence as shown in Table 1 and/or

a sequence (such as a CDRH2 sequence) comprising or consisting of a CDRH2 sequence as shown in Table 1 and/or

a sequence (such as a CDRH3 sequence) comprising or consisting of a CDRH3 sequence as shown in Table 1.

More suitably the polypeptide comprises

a sequence (such as a CDRH1 sequence) comprising or consisting of a CDRH1 sequence as shown in Table 1 and

a sequence (such as a CDRH2 sequence) comprising or consisting of a CDRH2 sequence as shown in Table 1 and

a sequence (such as a CDRH3 sequence) comprising or consisting of a CDRH3 sequence as shown in Table 1.

Suitably the polypeptide comprises

a sequence (such as a FWRH1 sequence) comprising or consisting of a sequence sharing 80% or greater sequence identity with a FWRH1 sequence as shown in Table 1 and/or

a sequence (such as a FWRH2 sequence) comprising or consisting of a sequence sharing 80% or greater sequence identity with a FWRH2 sequence as shown in Table 1 and/or

a sequence (such as a FWRH3 sequence) comprising or consisting of a sequence sharing 80% or greater sequence identity with a FWRH3 sequence as shown in Table 1 and/or

a sequence (such as a FWRH4 sequence) comprising or consisting of a sequence sharing 80% or greater sequence identity with a FWRH4 sequence as shown in Table 1.

In one embodiment the polypeptide comprises:

a sequence (such as a FWRH1 sequence) comprising or consisting of a sequence sharing 80% or greater sequence identity with a FWRH1 sequence as shown in Table 1 and/or

a sequence (such as a FWRH2 sequence) comprising or consisting of a sequence sharing 80% or greater sequence identity with a FWRH2 sequence as shown in Table 1 and/or

a sequence (such as a FWRH3 sequence) comprising or consisting of a sequence sharing 80% or greater sequence identity with a FWRH3 sequence as shown in Table 1 and/or

a sequence (such as a FWRH4 sequence) comprising or consisting of a sequence sharing 80% or greater sequence identity with a FWRH4 sequence as shown in Table 1.

More suitably the polypeptide comprises

a sequence (such as a FWRH1 sequence) comprising or consisting of a sequence sharing 90% or greater sequence identity with a FWRH1 sequence as shown in Table 1 and/or

a sequence (such as a FWRH2 sequence) comprising or consisting of a sequence sharing 90% or greater sequence identity with a FWRH2 sequence as shown in Table 1 and/or

a sequence (such as a FWRH3 sequence) comprising or consisting of a sequence sharing 90% or greater sequence identity with a FWRH3 sequence as shown in Table 1 and/or

a sequence (such as a FWRH4 sequence) comprising or consisting of a sequence sharing 90% or greater sequence identity with a FWRH4 sequence as shown in Table 1.

More suitably the polypeptide comprises

a sequence (such as a FWRH1 sequence) comprising or consisting of a FWRH1 sequence as shown in Table 1 and/or

a sequence (such as a FWRH2 sequence) comprising or consisting of a FWRH2 sequence as shown in Table 1 and/or

a sequence (such as a FWRH3 sequence) comprising or consisting of a FWRH3 sequence as shown in Table 1 and/or

a sequence (such as a FWRH4 sequence) comprising or consisting of a FWRH4 sequence as shown in Table 1.

More suitably the polypeptide comprises

a sequence (such as a FWRH1 sequence) comprising or consisting of a FWRH1 sequence as shown in Table 1 and

a sequence (such as a FWRH2 sequence) comprising or consisting of a FWRH2 sequence as shown in Table 1 and

a sequence (such as a FWRH3 sequence) comprising or consisting of a FWRH3 sequence as shown in Table 1 and

a sequence (such as a FWRH4 sequence) comprising or consisting of a FWRH4 sequence as shown in Table 1.

Suitably the polypeptide comprises three complementarity determining regions (CDRH1-CDRH3). Suitably, the polypeptide comprises four framework regions (FWRH1-FWRH4).

In one embodiment there is provided a polypeptide comprising or consisting of a sequence sharing 80% or greater, more suitably 90% or greater, sequence identity with any immunoglobulin heavy chain variable domain (VH) sequence as shown in Table 1 (i.e. from N- to C-terminus, the combined sequence of FWRH1, CDRH1, FWRH2, CDRH2, FWRH3, CDRH3, FWRH4, for a single row) or Table 2. More suitably the polypeptide comprises or consists of an immunoglobulin heavy chain variable domain (VH) sequence as shown in Table 1 (i.e. from N- to C-terminus, the combined sequence of FWRH1, CDRH1, FWRH2, CDRH2, FWRH3, CDRH3, FWRH4, for a single row) or Table 2.

Suitably the polypeptide is an antibody, such as an antibody which belongs to the isotype subclass IGHA1, IGHA2 or IGHG1. Alternatively, the polypeptide is an antibody fragment, such as a F(ab′)₂, an Fd, an Fv, an scFv, a VH, or a VHH.

Suitably the polypeptide binds to the spike protein (S protein) of SARS-CoV-2. More suitably the polypeptide binds to the S1 or S2 domain of the spike protein (S protein), such as the S1 domain of the spike protein (S1 protein).

An antibody fragment as used herein refers to a portion of an antibody that binds to a target. Examples of binding fragments encompassed within the term include a Fab, a F(ab′)₂, an Fd, an Fv, an scFv, a VH, or a VHH.

Suitably the polypeptide comprises light chain CDRs (i.e. CDRL1, CDRL2, CDRL3). More suitably the polypeptide comprises light chain CDRs and framework regions (i.e. FWRL1, CDRL1, FWRL2, CDRL2, FWRL3, CDRL3 and FWRL4). More suitably the polypeptide is an antibody comprising both heavy and light chains. Suitably the light chain CDRs and/or frameworks and/or light chains are any one or more of those disclosed in Xue et al. Biochem Biophys Res Commun. 515(3):481-486, (2019).

Suitably, the polypeptide of the invention is isolated. An “isolated” polypeptide is one that is removed from its original environment. For example, a naturally-occurring polypeptide of the invention is isolated if it is separated from some or all of the coexisting materials in the natural system.

In one embodiment there is provided a pharmaceutical composition comprising the polypeptide and one or more pharmaceutically acceptable diluents or carriers. Suitably the composition comprises at least one further, different polypeptide according to any preceding claim. Suitably the composition comprises at least one further active agent.

In one embodiment the polypeptide or pharmaceutical composition is for use in suppressing or treating a disease or disorder mediated by infection of SARS-CoV-2, such as COVID-19, or for providing prophylaxis to a subject at risk of infection of SARS-CoV-2, such as COVID-19. In one embodiment there is provided a method of suppressing or treating a disease or disorder mediated by infection of SARS-CoV-2, such as COVID-19 or for providing prophylaxis to a subject at risk of infection of SARS-CoV-2, such as COVID-19, comprising administering to a person in need thereof a therapeutically effective amount of the polypeptide or pharmaceutical composition.

In one embodiment there is provided a polynucleotide encoding a polypeptide sequence disclosed in Table 1 or Table 2. In one embodiment there is provided a polynucleotide encoding an immunoglobulin heavy chain variable domain recited in Table 1 or Table 2. In one embodiment there is provided a vector comprising the polynucleotide.

The present invention will now be further described by means of the following non-limiting example.

### Equivalents and Scope

While various invention embodiments have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of a group is present in, employed in, or otherwise relevant to a given product or process. The invention includes embodiments in which more than one, or all group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1

### COVID-19 Disease Samples

Blood samples were collected from n=19 patients admitted to hospital with acute COVID-19 pneumonia. The mean age of patients was 50.2 (SD 18.5) years and 13 (68%) were male. All patients had a clinical history consistent with COVID-19 and typical radiological changes. Seventeen patients had a confirmatory positive PCR test for SARS-CoV-2. The patients experienced an average of 11 days (range 4-20) of symptoms prior to the day on which the blood sample was collected. Nine of the patients were still requiring hospital care but not oxygen therapy on day of sample collection (WHO Ordinal Scale Score 3), while eight were hospitalised requiring oxygen by conventional mask or nasal prongs (WHO Ordinal Scale Score 4) and two were hospitalised with severe COVID-19 pneumonia requiring high-flow nasal oxygen (WHO Ordinal Scale Score 5). On the day of sample collection, the direct clinical care team considered two patients to be deteriorating, four improving and the remaining thirteen were clinically stable.

### SARS-CoV-2 Infection Results in a Stereotypic B Cell Response

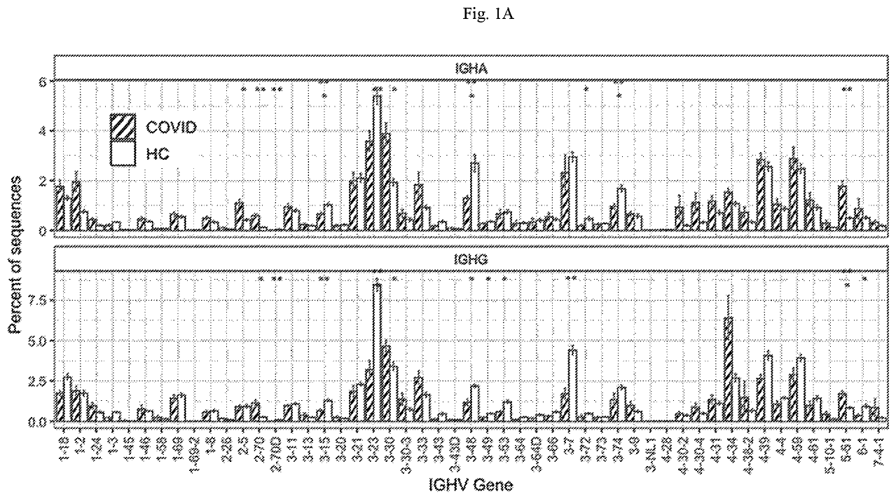

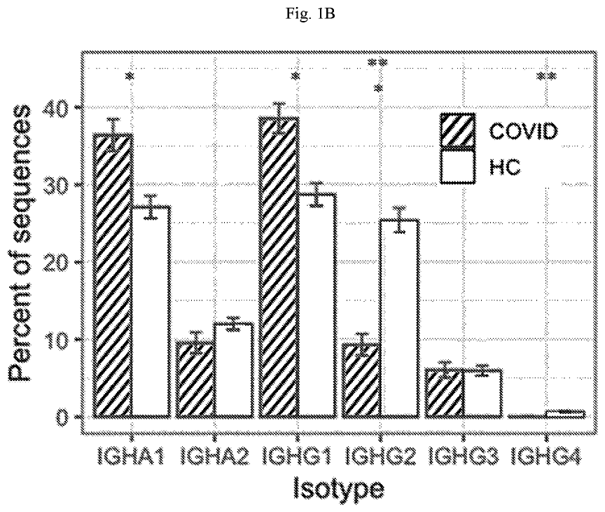

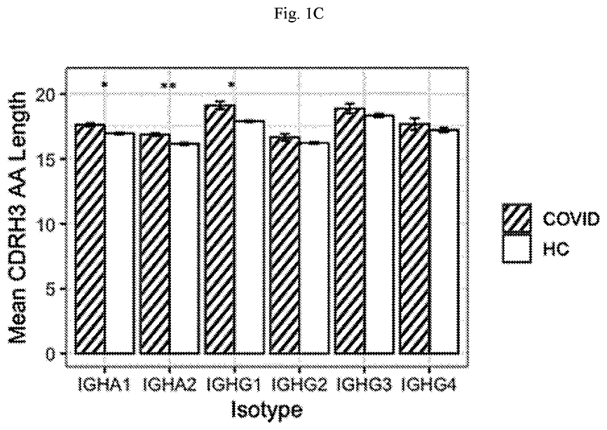

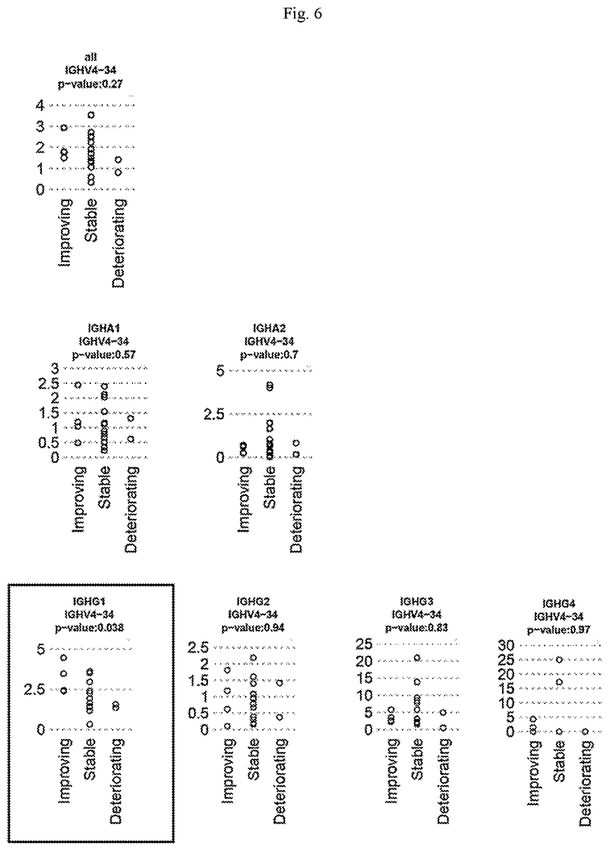

IGHA and IGHG BCR sequencing yielded on average 135,437 unique sequences, and 23,742 clonotypes per sample (Table 3). To characterise the B cell response in COVID-19, we compared this BCR repertoire data to BCR repertoire data from healthy controls obtained in a separate study¹⁵. Comparing IGHV gene segment usage revealed a significantly different IGHV gene usage in COVID-19 patients compared to the healthy controls, most notably with increases in the usage of IGHV2-5 (2.6×IGHA, 1.0×IGHG increase), IGHV2-70 (4.6×IGHA, 4.1×IGHG increase), IGHV3-30 (2.0×IGHA, 1.4×IGHG increase), IGHV5-51 (3.5×IGHA, 2.0×IGHG increase), and IGHV4-34 (1.4×IGHA, 2.4×IGHG increase) in the COVID-19 patients (FIG. 1A). All of these V gene segments have been previously observed in SARS-CoV-1 or SARS-CoV-2 specific antibodies¹⁶. IGHV4-34 has been shown to bind both autoantigens¹⁷ and commensal bacteria¹⁸ and has been associated with SLE¹⁹. Our data extends this, showing that the proportion of sequences containing the autoreactive AVY & NHS sequence motifs within the IGHV region is significantly more frequent in improving COVID-19 patients compared to stable or deteriorating COVID-19 patients, specifically in the IGHG1 isotype subclass (p-value=0.038; FIG. 6).

Comparing isotype subclasses showed a significant increase in the relative usage of IGHA1 and IGHG1 in COVID-19 patients (FIG. 1B)—these are the two first isotype subclasses that are switched to upon activation of IGHM²⁰. There was also an increase in the mean CDRH3 length of the BCRs in the COVID-19 patients, that was most pronounced in the IGHA1, IGHA2 and IGHG1 isotype subclasses (FIG. 1C).

### SARS-CoV-2 Infection Stimulates Both Naïve and Memory Responses

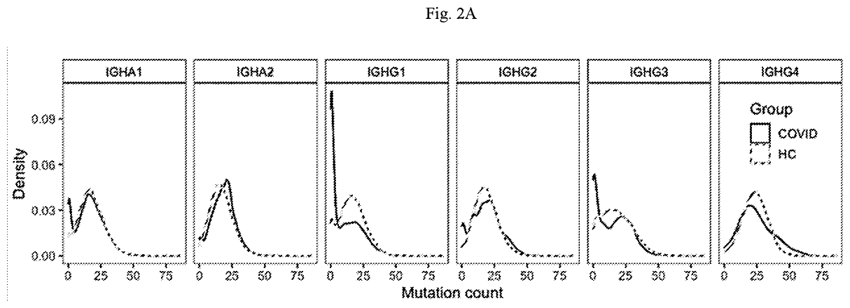

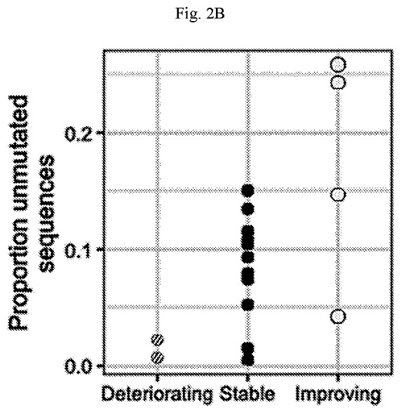

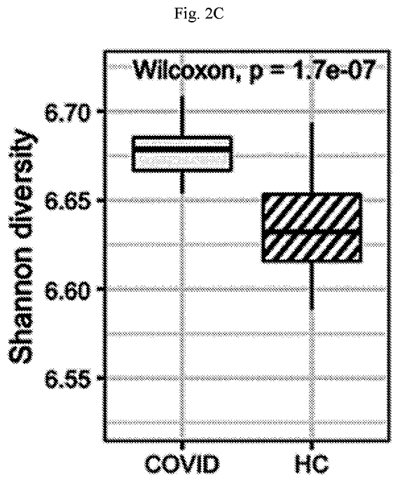

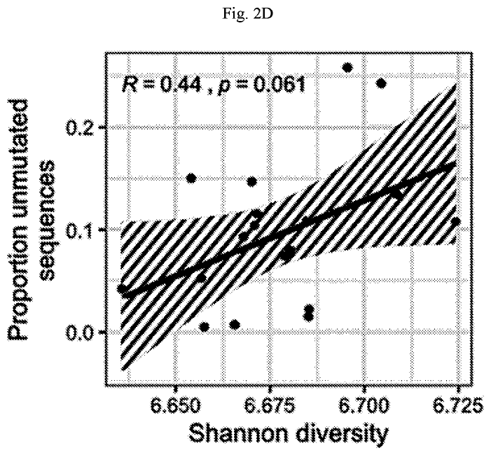

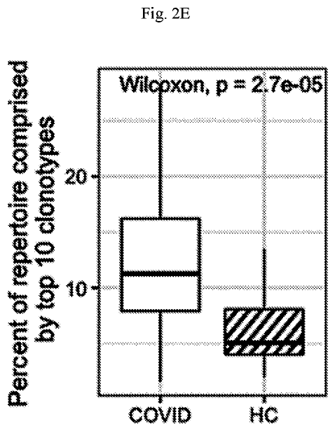

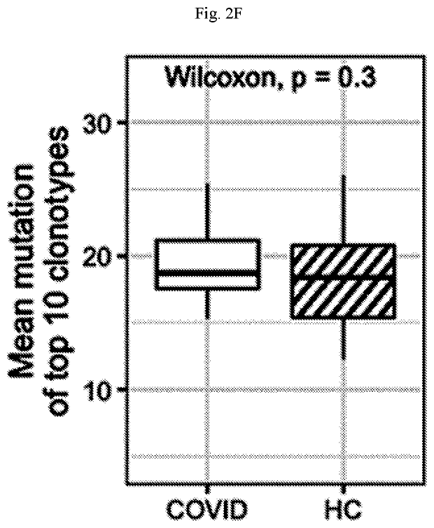

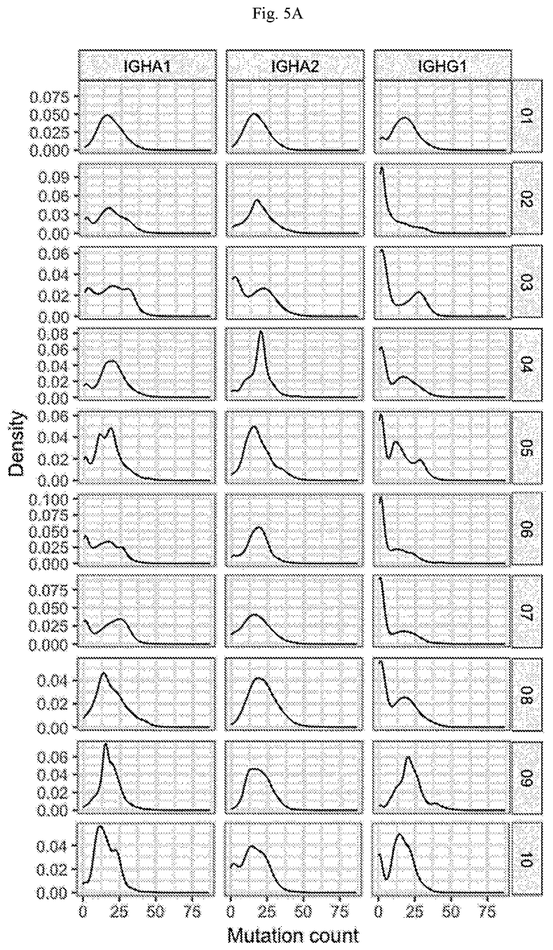

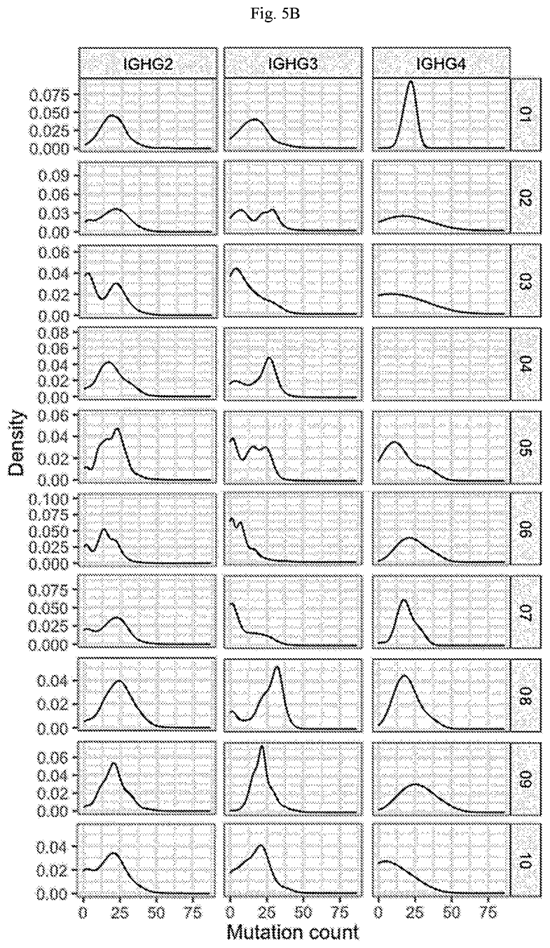

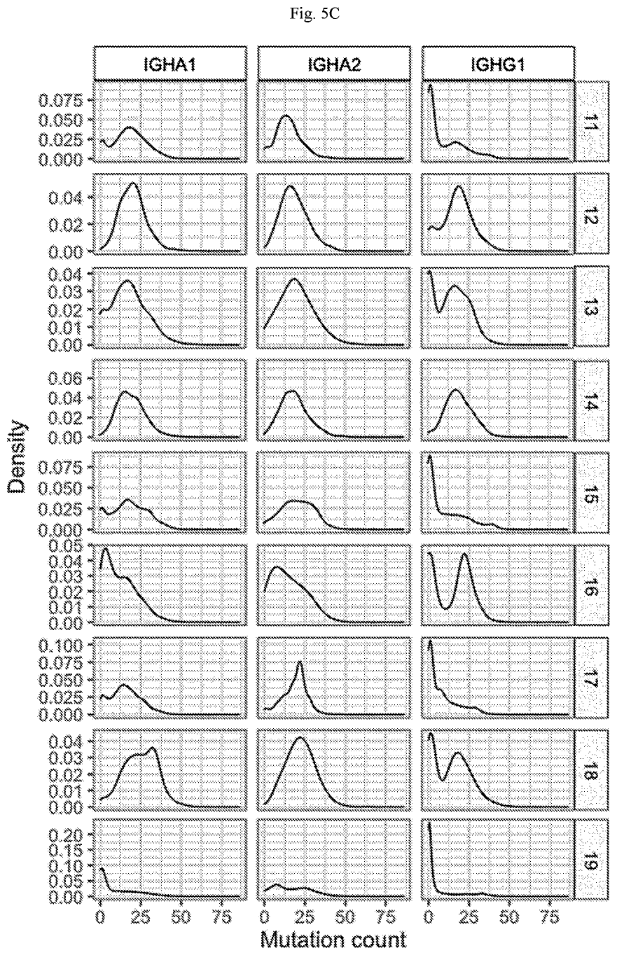

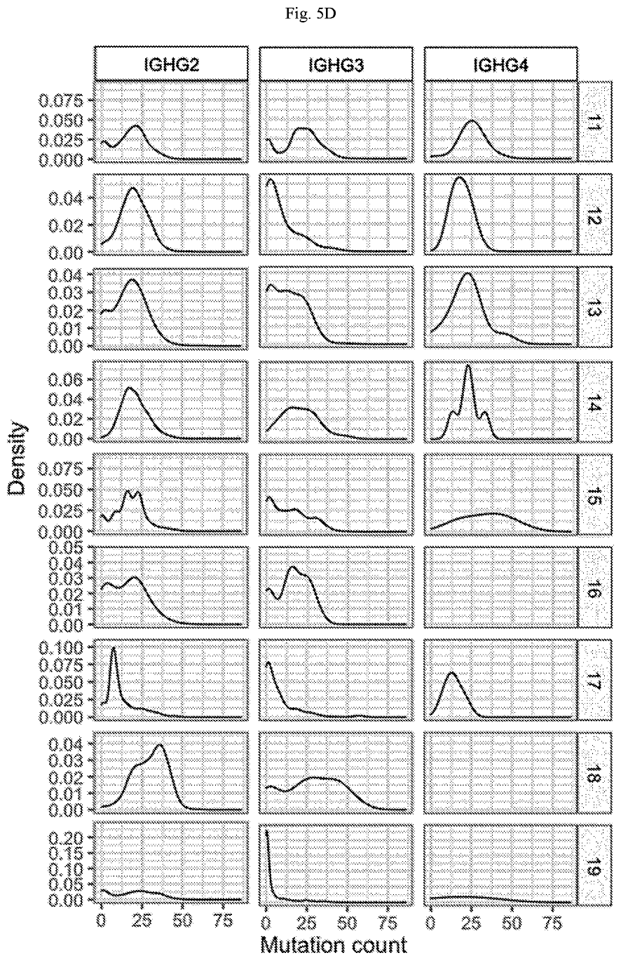

To further investigate the COVID-19-specific B cell response, we analysed the characteristics of the BCR sequences that are consistent with recent B cell activation—somatic hypermutation, and clonal expansion. In healthy controls, for class-switched sequences, there is a clear unimodal distribution of sequences with different numbers of mutations, and a mean mutation count across IGHA and IGHG isotypes of 17.6 (FIG. 2A). In the COVID-19 samples, the mean mutation count was 14.4, and there was a bimodal distribution with a separate peak of sequences with no mutations. This bimodal distribution was most pronounced in the IGHG1, IGHG3, and IGHA1 isotype subclasses, corresponding to the increased isotype usages. Such a distribution is consistent with an expansion of recently class-switched B cells that have yet to undergo somatic hypermutation. There was considerable variation between participants in the proportion of unmutated sequences (FIG. 5A, FIG. 5B, FIG. 5C, and FIG. 5D), which had no significant correlation with the number of days since symptom onset (R=0.09, p=0.72), but was lower in the deteriorating compared to improving patients (FIG. 2B)

To investigate differential clonal expansion between patients, the Shannon diversity index of each repertoire was calculated (while accounting for differences in read depth through subsampling). A more diverse repertoire is indicative of a greater abundance of different clonal expansions. The BCR repertoires of the COVID-19 patients were significantly more diverse than the BCR repertoires of the healthy controls (FIG. 2C); this increase in diversity was positively correlated with an increased proportion of unmutated sequences (R=0.44, p=0.061; FIG. 2D). Interestingly, when we investigated the largest clonal expansions, despite having a more diverse repertoire, the largest clonal expansions in the COVID-19 samples were larger than in the healthy controls (FIG. 2E). These large clonal expansions were also highly mutated and had similar levels of mutation between the COVID-19 samples and the healthy controls (FIG. 2F).

### Sequence Convergence can be Used to Identify Putative SARS-CoV-2 Specific Antibodies

Given the skewing of the B cell response in the COVID-19 patients to specific IGHV genes, we next investigated whether the same similarity was also seen on the BCR sequence level between different participants. Such convergent BCR signatures have been observed in response to other infectious diseases²¹, and may be used to identify disease-specific antibody sequences.

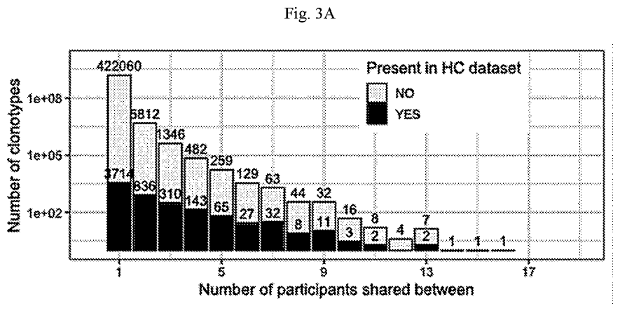

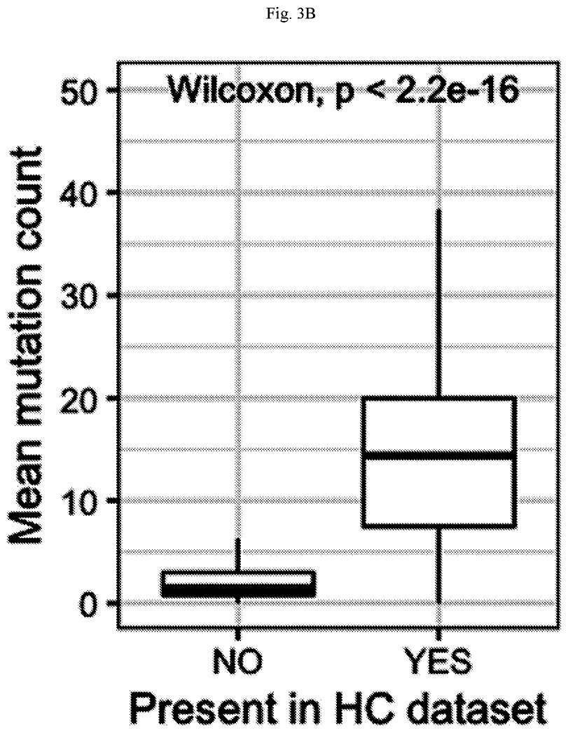

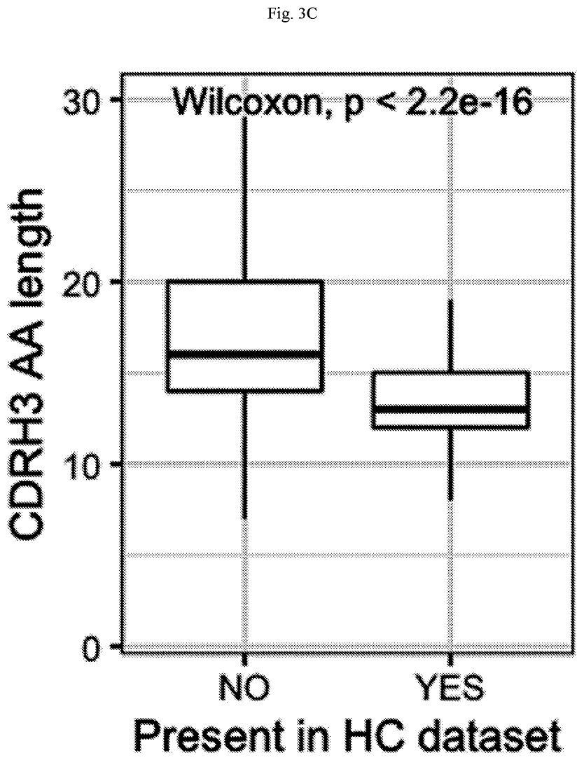

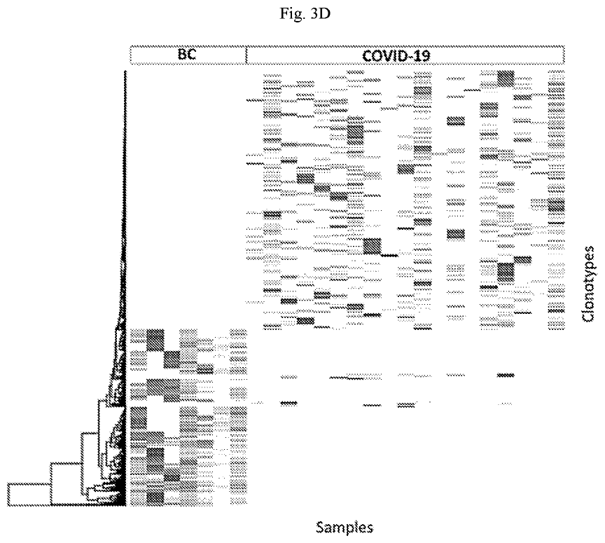

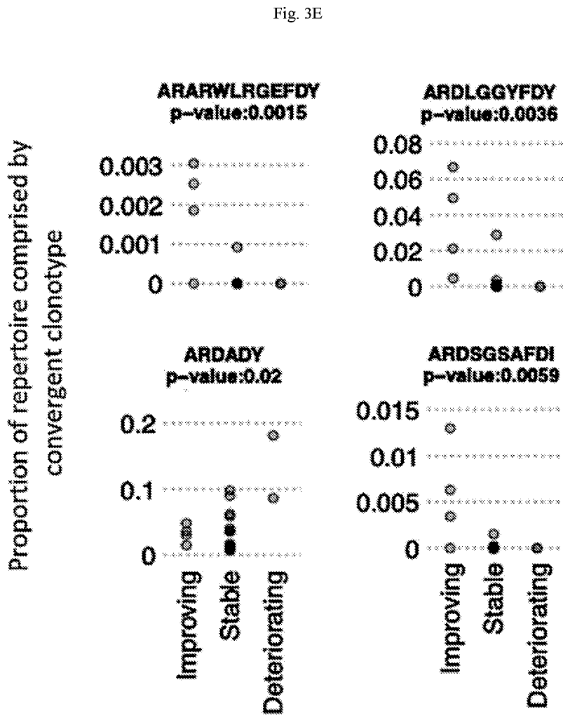

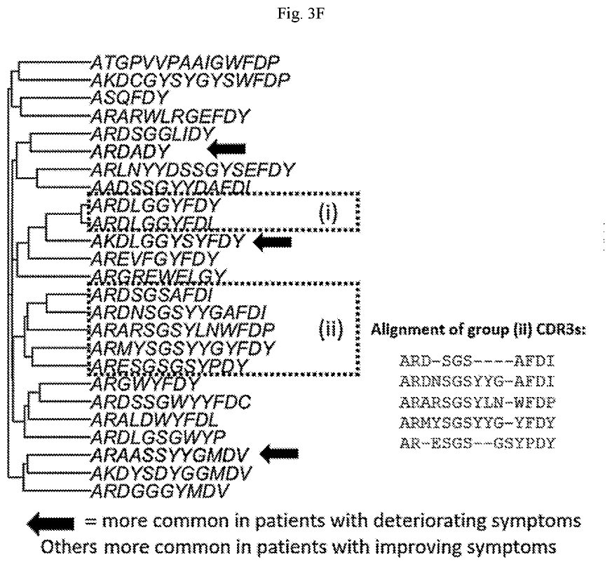

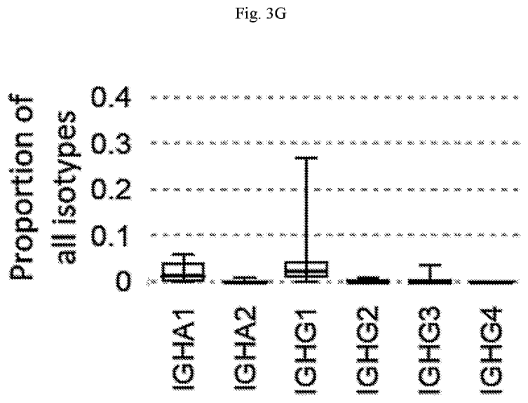

Of the 435,420 total clonotypes across all the COVID-19 patients, 9,646 (2.2%) were shared between at least two of the participants (FIG. 3A). As convergence could occur by chance or be due to an unrelated memory response from commonly encountered pathogens, the healthy control dataset was used to subtract irrelevant BCR sequences. Of the 9,646 convergent clonotypes, 1,442 (14.9%) were also present in at least one of the 40 healthy control samples. As expected, of the convergent clonotypes that were also present in the healthy control samples, the mean mutation count was significantly greater (FIG. 3B), and the mean CDRH3 length significantly shorter (FIG. 3C) than the convergent clonotypes that were unique to the COVID-19 patients.

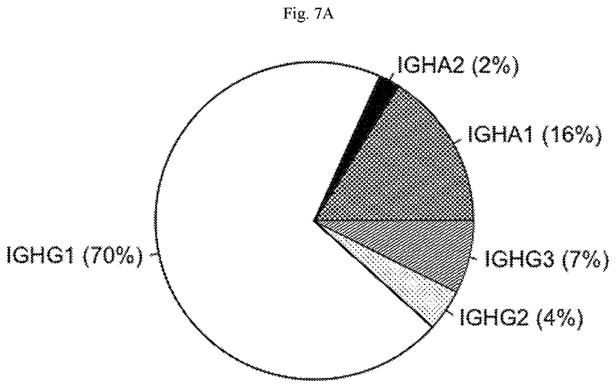

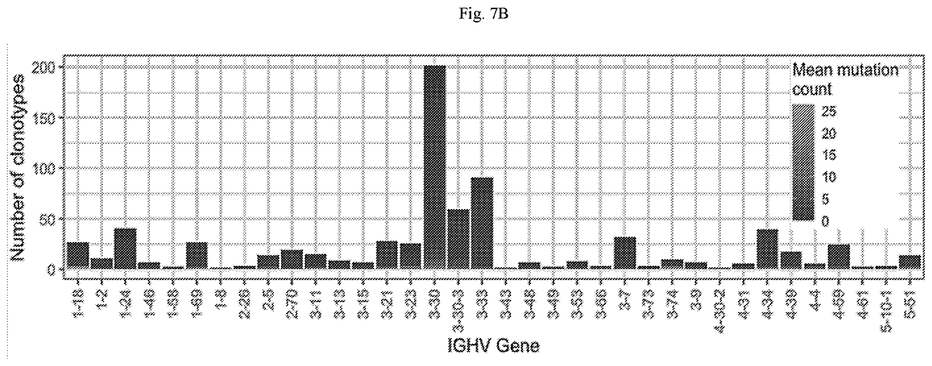

To identify a set of SARS-CoV-2-specific antibody sequences with high confidence, we identified 777 convergent clonotypes that were shared between at least four of the COVID-19 patients (see Tables 1 and 2, which also include further convergent clonotypes from another set of samples), but not seen in the healthy controls. In parallel, for a comparison of convergent signatures, we performed the same analysis on a cohort of seven metastatic breast cancer patient biopsy samples²², which identified 469 convergent clonotypes. These convergent clonotypes were highly specific to each disease cohort (FIG. 3D). The 777 COVID-19 convergent clonotypes had low mutation levels, with a mean mutation count of 2, and only 51 clonotypes with a mean mutation greater than 5. The sequences within the convergent clonotypes were primarily of the IGHG1 (70%) and IGHA1 (16%) subclasses (FIG. 7A). The convergent clonotypes used a diversity of IGHV gene segments, with IGHV3-30, IGHV3-30-3 and IGHV3-33 as the most highly represented (FIG. 7B). This IGHV gene usage distribution differs between that of the total repertoire, and IGHV3-30 is also the most highly used IGHV gene in the CoV-AbDab¹⁶.

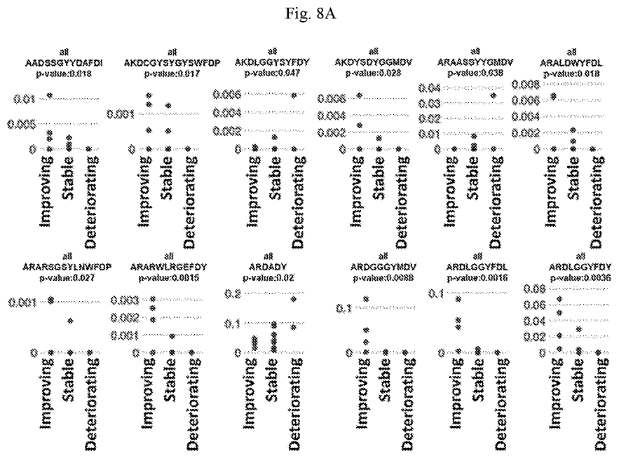

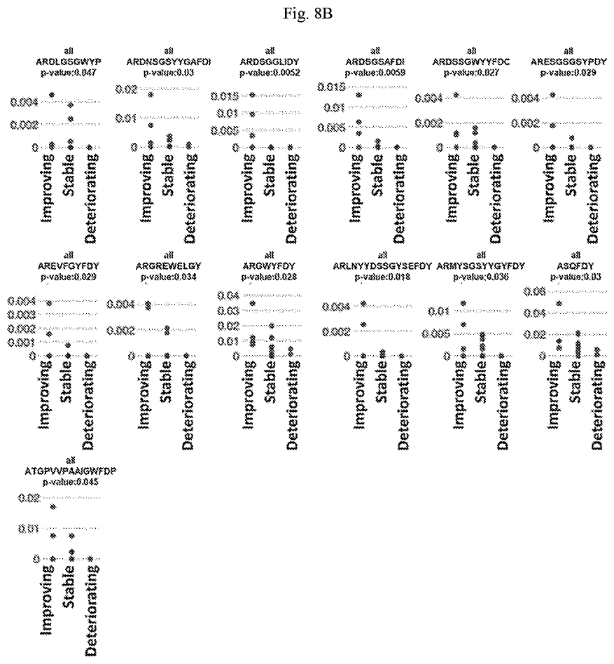

We next tested whether these convergent clonotypes correlated with disease severity. Indeed, 25 of these convergent clonotypes were found to associate with clinical symptoms after correcting for multiple testing, of which 22 were observed at a significantly higher frequency in improving patients (FIG. 3E, FIG. 8A, and FIG. 8B). This is a significantly higher proportion associated with clinical symptoms compared to that expected by chance (p-value=0.018 by random permutations of labels). Interestingly, some of these clonotypes are common in patients comprising >0.1% of a patient's repertoire. Furthermore, the convergent clonotypes that are associated with clinical symptoms cluster together (FIG. 3F) and are found primarily in the IGHA1 and IGHG1 isotypes (FIG. 3G).

BCR Sequence Convergence Signatures are Shared Between Different COVID-19 Studies in Different Locations and from Different Anatomical Sites

To further explore whether the convergent clonotypes observed in our study were indeed disease specific, and to determine whether such convergence was common across studies and geographic regions, we compared these 777 convergent clonotypes to public B cell datasets.

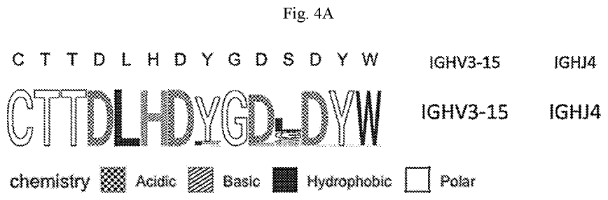

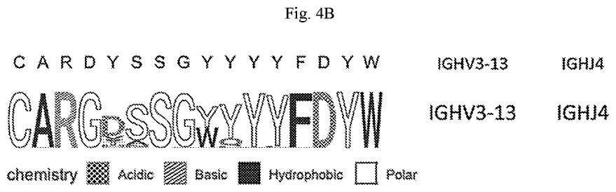

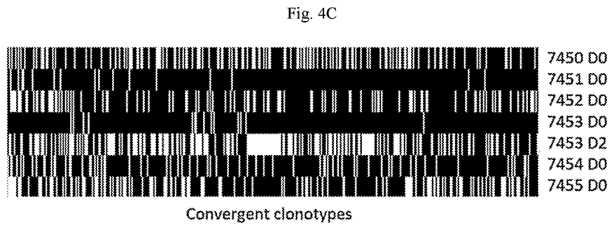

First, we compared our data to RNAseq data of bronchoalveolar lavage fluid obtained from five of the first infected patients in Wuhan, China²³. These samples were obtained for the purpose of metagenomic analyses to identify the aetiological agent of the novel coronavirus but were re-analysed to determine whether we could extract any transcripts from BCRs. From the 10,038,758 total reads, we were able to identify 16 unique CDR3 AA sequences (Table 4). Of these, one had an exact AA match to a clonotype in our data and shared the same V gene segment (IGHV3-15), and J gene segment (IGHJ4) usage (FIG. 4A). This clonotype had a CDRH3 AA length of 12, so such a match is unlikely to occur due to chance alone. The clonotype contained 699 total sequences and was convergent between 8 of our 19 COVID-19 patients, but not present in the healthy controls. The clonotype was highly diverse, and the sequences had evidence of low mutation from germline, with a mean mutation count over all sequences of 4.8 (FIG. 7B).

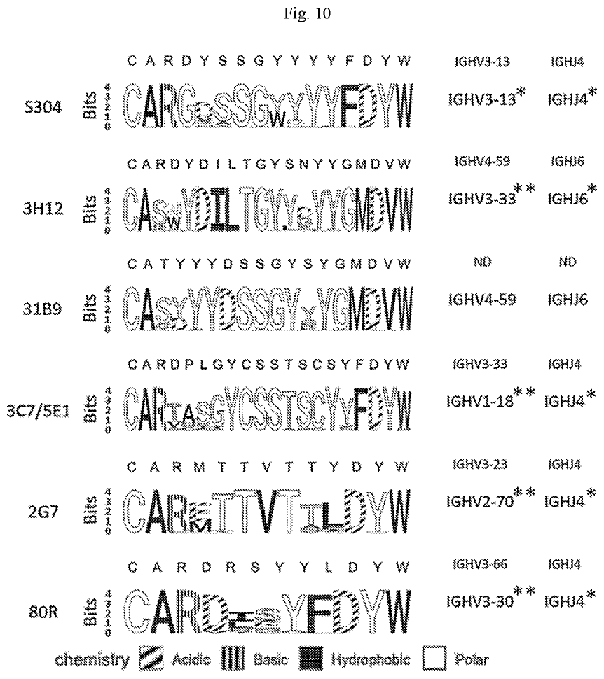

Next, we compared our 777 convergent clonotypes to CoV-AbDab—the Coronavirus Antibody Database [accessed 10 May 2020]¹⁶. At the time of access, this database contained 80 non-redundant CDRH3 sequences from published and patented antibodies proven to bind SARS-CoV-1 and/or SARS-CoV-2. We found 6 of our clonotypes to have high CDRH3 homology to the antibodies in CoV-AbDab (FIG. 4B and FIG. 10). The most striking similarity was to S304, a previously described SARS-CoV-1 and SARS-CoV-2 receptor-binding domain antibody able to contribute to viral neutralisation²⁴. One of the 777 convergent clonotypes contained sequences with an exact CDRH3 AA sequence match and utilised the same IGHV and IGHJ germline gene segments to S304. This clonotype was convergent across 6 patients and had a mean mutation count of 1.1.

Finally, we compared our data to a publicly available BCR deep sequencing dataset from six COVID-19 patients from Stanford, USA. 405 of our 777 convergent clonotypes matched to sequences in this dataset (FIG. 4C), showing the high level of convergence between studies. The average number of clonotype matches to the Stanford COVID-19 patient repertoires was 95, but this varied considerably between patients and timepoints. Two of the six patients were seronegative at the day of sampling (7451 and 7453), and these two patients had the fewest clonotype matches (16 and 14 respectively). Patient 7453 had an additional sample taken two days later (following seroconversion), and at this point had a large increase in the number of clonotype matches to 204.

### Supplementary Information

TABLE 3
Summary of number of unique sequences, and number
of clonotypes obtained for each COVID-19 patient

| Participant ID | Unique BCR Sequences | Clonotypes |
|---|---|---|
| 1 | 47878 | 15456 |
| 2 | 257570 | 53168 |
| 3 | 33099 | 9616 |
| 4 | 37138 | 10754 |
| 5 | 198732 | 20036 |
| 6 | 233283 | 26181 |
| 7 | 51305 | 22276 |
| 8 | 39303 | 9391 |
| 9 | 221870 | 18278 |
| 10 | 54645 | 9255 |
| 11 | 202896 | 41132 |
| 12 | 31035 | 6791 |
| 13 | 40995 | 14782 |
| 14 | 171231 | 21373 |
| 15 | 280310 | 36446 |
| 16 | 29620 | 8736 |
| 17 | 253037 | 34805 |
| 18 | 60316 | 15068 |
| 19 | 329055 | 77557 |

TABLE 4
CDRH3 AA sequences identified from bronchoalveolar RNAseq data

| bestVHit | bestJHit | aaSeqCDR3 | SEQ ID NO |
|---|---|---|---|
| IGHV2-26 | IGHJ3 | CARDSGRHLGPFDIW | 1999 |
| IGHV1-2 | IGHJ3 | CATPYYYDGGLDAFDIW | 2000 |
| IGHV3-74 | IGHJ5 | CARDLSRTNWFDPW | 2001 |
| IGHV3-15 | IGHJ4 | CTTDLHDYGDSDYW | 2002 |
| IGHV3-15 | IGHJ4 | CTTDFGGMITFGGVLRRI | 2003 |
| IGHV3-21 | IGHJ4 | CARAQSRGGYDSFFDFW | 2004 |
| IGHV3-21 | IGHJ4 | CGRGGPGTGIDYW | 2005 |
| IGHV4-59 | IGHJ5 | CARGGQYNNWFAPW | 2006 |
| IGHV3-74 | IGHJ5 | CVRDLSRTNWFDPW | 2007 |
| IGHV3-15 | IGHJ4 | YTRDLHDYGDSDYW | 2008 |
| IGHV3-23 | IGHJ3 | CAKIPSFLSDYDVHPNDAIDIW | 2009 |
| IGHV5-10-1 | IGHJ4 | CARHPQGAQFSNLGTYYFDYW | 2010 |
| IGHV4-59 | IGHJ4 | CARDGEYGGLAMW | 2011 |
| IGHV5-51 | IGHJ6 | CARPGTYYDILTGYSNHGMDVW | 2012 |
| IGHV4-39 | IGHJ5 | CARHASFRGTNYNWFDPW | 2013 |
| IGHV3-53 | IGHJ5 | CARDTSTEDVAWWFDPW | 2014 |

The CDRH3 identified in our SARS-CoV-2 patient dataset is SEQ ID NO: 2002.

### Discussion

We have used deep sequencing of the BCR heavy chain repertoire to evaluate the B cell responses of 19 individuals with COVID-19. In agreement with previous studies, there was a skewing of the repertoire in the response to SARS-CoV-2 infection, with an increased use of certain V genes, and an increase in the proportion of antibodies with longer CDRH3s, and an altered isotype subclass distribution¹⁴. The significantly increased usage of IGHA1 observed in the COVID-19 patients is in line with mucosal responses, where the longer hinge in IGHA1 compared to IGHA2 may offer advantages in antigen recognition by allowing higher avidity bivalent interactions with distantly spaced antigens.

As anticipated, given the novel nature of the virus, that SARS-CoV-2 infection largely stimulated a characteristically naïve response, rather than a reactivation of pre-existing memory B cells—(1) there was an increased prevalence of unmutated antigen-experienced class-switched BCR sequences, (2) an increase in the diversity of class-switched IGHA and IGHG BCRs, and (3) an increase in the usage of isotype subclasses that are associated with viral immunity. These observations are consistent with an increase in the frequency of recently activated B cells in response to SARS-CoV-2. In addition to the naïve response, there was also evidence of a proportion of the response arising from memory recall. In the COVID-19 patients, the largest clonal expansions were highly mutated, equivalent to the level observed in healthy control cohort. Such a secondary response to SARS-CoV-2 has been previously observed²⁵, and may be due to recall of B cells activated in response to previously circulating human coronaviruses, as recently highlighted^{26,27}.

We observed a potential relationship between repertoire characteristics and disease state, with improving patients showing a tendency towards a higher proportion of unmutated sequences. The increased prevalence of autoreactive IGHV4-34 sequences in improving COVID-19 patients compared to stable or deteriorating COVID-19 patients potentially suggests a role for natural or autoreactive antibodies in resolving infection and lower risk of pathology. However, this will need to be confirmed using larger sample cohorts. There is a clear need to expand on these findings by deepening the data pool and gathering more clinical data to aid understanding of the differences between individuals that respond with mild versus severe disease and have different recovery patterns. Building upon these observations could help to inform the future development of diagnostic assays to monitor and predict the progression of disease in infected patients.

A large number (777) of highly convergent clonotypes unique to COVID-19 were identified (see Table 1 and Table 2, which also include further convergent clonotypes from a separate set of samples). Our approach of subtracting the convergent clonotypes also observed in healthy controls¹⁵, allowed us to identify convergence specific to the disease cohort. The unbiased nature of the BCR repertoire analysis approach means that, whilst these convergent clonotypes are likely to include many antibodies to the spike protein and other parts of the virus they may also include other protective antibodies, including those to host proteins. It is expected that the heavy chains we have identified, and components of these heavy chains, will find utility in the treatment, prevention and diagnosis of COVID-19. Furthermore, characterisation of the heavy chains we have identified, coupled with matched light chains to generate functional antibodies will permit analysis of the binding sites and neutralising potential of these antibodies. The report that plasma derived from recently recovered donors with high neutralising antibody titres can improve the outcome of patients with severe disease²⁸, supports the hypotheses that intervention with a therapeutic antibody has the potential to be an effective treatment. A manufactured monoclonal antibody or combination of antibodies would also provide a simpler, scalable and safer approach than plasma therapy.

Sequence convergence between our 777 convergent clonotypes with heavy chains from published and patented SARS-CoV-1 and SARS-CoV-2 antibodies¹⁶ supports several observations. Firstly, it demonstrates that our approach of finding a convergent sequence signature is a useful method for enriching disease-specific antibodies, as we find matches to known SARS-CoV spike-binding antibodies. Secondly, it shows that the clonotypes observed in response to SARS-CoV-2 overlap with those to SARS-CoV-1, presumably explained by the relatively high homology of the two related viruses 3. Indeed, here we show that there is an overrepresentation of clonotypes that correlate with patient clinical symptoms than is expected by chance, and these BCR sequences are associated with the dominant IgA1 and IgG1 responses. Finally, it shows that the convergence extends beyond our UK COVID-19 disease cohort.

Further evidence for convergence extending beyond our disease cohort came from the comparisons of our 777 convergent clonotypes to deep sequencing datasets from China²³ and the USA¹⁴. The dataset from the USA is also from BCR sequencing of the peripheral blood of COVID-19 patients, and here we found matches to 405 of our 777 clonotypes. The dataset from China was from total RNA sequencing of the bronchoalveolar lavage fluid of SARS-CoV-2 infected patients. Only 16 unique CDRH3 sequences could be identified in this whole dataset, but one of them matched a convergent clonotype in the current study, showing that convergence can be seen both between different locations, and different sample types. We believe that the identification of such high BCR sequence convergence between geographically distinct and independent datasets could be highly significant and validates the disease association of the clonotypes, as well as the overall approach.

In summary, our BCR repertoire analysis provides information on the specific nature of the B cell response to SARS-CoV-2 infection. The information generated has the potential to facilitate the treatment of COVID-19 by supporting diagnostic approaches to predict the progression of disease, informing vaccine development and enabling the development of therapeutic antibody treatments and prophylactics.

### Materials and Methods

### Clinical Information Gathering

Peripheral blood was obtained from patients admitted with acute COVID-19 pneumonia to medical wards at Barts Health NHS Trust, London, UK, after informed consent by the direct care team (NHS HRA RES Ethics 19/SC/0361). Venous blood was collected in EDTA Vacutainers (BD). Patient demographics and clinical information relevant to their admission were collected by members of the direct care team, including duration of symptoms prior to blood sample collection. Current severity was mapped to the WHO Ordinal Scale of Severity. Whether patients at time of sample collection were clinically Improving, Stable or Deteriorating was subjectively determined by the direct clinical team prior to any sample analysis. This determination was primarily made on the basis of whether requirement for supplemental oxygen was increasing, stable, or decreasing comparing current day to previous three days.

### Sample Collection and Initial Processing

Blood samples were centrifuged at 150×g for 15 minutes at room temperature to separate plasma. The cell pellet was resuspended with phosphate-buffered saline (PBS without calcium and magnesium, Sigma) to 20 ml, layered onto 15 ml Ficoll-Paque Plus (GE Healthcare) and then centrifuged at 400×g for 30 minutes at room temperature without brake. Mononuclear cells (PBMCs) were extracted from the buffy coat and washed twice with PBS at 300×g for 8 min. PBMCs were counted with Trypan blue (Sigma) and viability of >96% was observed. 5×10⁶ PBMCs were resuspended in RLT (Qiagen) and incubated at room temperature for 10 min prior to storage at −80° C. Consecutive donor samples with sufficient RLT samples progressed to RNA preparation and BCR preparation and are included in this manuscript.

Metastatic breast cancer biopsy samples were collected and RNA extracted as part of a previously reported cohort²².

### RNA Prep & BCR Sequencing

Total RNA from 5×10⁶ PBMCs was isolated using RNeasy kits (Qiagen). First-strand cDNA was generated from total RNA using SuperScript RT IV (Invitrogen) and IgA and IgG isotype specific primers²⁹ including UMIs at 50° C. for 45 min (inactivation at 80° C. for 10 min).

The resulting cDNA was used as template for High Fidelity PCR amplification (KAPA, Roche) using a set of 6 FR1-specific forward primers²⁹ including sample-specific barcode sequences (6 bp) and a reverse primer specific to the RT primer (initial denaturation at 95° C. for 3 min, 25 cycles at 98° C. for 20 sec, 60° C. for 30 sec, 72° C. for 1 min and final extension at 72° C. for 7 min). The amount of Ig amplicons (˜450 bp) was quantified by TapeStation (Beckman Coulter) and gel-purified.

Dual-indexed sequencing adapters (KAPA) were ligated onto 500 ng amplicons per patient using the HyperPrep library construction kit (KAPA) and the adapter-ligated libraries were finally PCR-amplified for 3 cycles (98° C. for 15 sec, 60° C. for 30 sec, 72° C. for 30 sec, final extension at 72° C. for 1 min). Pools of 10 and 9 libraries were sequenced on an Illumina MiSeq using 2×300 bp chemistry.

### Sequence Processing

The Immcantation framework was used for sequence processing^{30,31} Briefly, paired-end reads were joined based on a minimum overlap of 20 nt, and a max error of 0.2, and reads with a mean phred score below 20 were removed. Primer regions, including UMIs and sample barcodes, were then identified within each read, and trimmed. Together, the sample barcode, UMI, and constant region primer were used to assign molecular groupings for each read. Within each grouping, usearch³², was used to subdivide the grouping, with a cutoff of 80% nucleotide identity, to account for randomly overlapping UMIs. Each of the resulting groupings is assumed to represent reads arising from a single RNA. Reads within each grouping were then aligned, and a consensus sequence determined.

For each processed sequence, IgBlast³³ was used to determine V, D and J gene segments, and locations of the CDRs and FWRs. Isotype was determined based on comparison to germline constant region sequences. Sequences annotated as unproductive by IgBlast were removed. The number of mutations within each sequence was determined using the shazam R package³¹.

Sequences were clustered to identify those arising from clonally related B cells; a process termed clonotyping. Sequences from all samples were clustered together to also identify convergent clusters between samples. Clustering was performed using a previously described algorithm³⁴. Clustering required identical V and J gene segment usage, identical CDRH3 length, and allowed 1 AA mismatch for every 10 AAs within the CDRH3. Cluster centers were defined as the most common sequence within the cluster. Lineages were reconstructed from clusters using the alakazam R package³⁵. The similarity tree of the convergent clonontype CDR3 sequences was generated through a kmer similarity matrix between sequences in R.

### Public Healthy Control Data Processing

The healthy control BCR sequence dataset used here has been described previously¹⁵. Only samples from participants aged 10 years or older, and from peripheral blood were used, resulting in a mean age of 28 (range: 11-51). Furthermore, only class-switched sequences were considered.

### Public SARS-CoV-2 Bronchoalveolar Lavage RNAseq Data Processing

The bronchoalveolar lavage data comes from a previously published study of SARS-CoV-2 infection²³ with data available under the PRJNA605983 BioProject on NCBI. MIXCR v3.0.3 was used, with default settings, to extract reads mapping to antibody genes from the total RNASeq data³⁶.

### Public CoV-AbDab Data Processing

All public CDRH3 AA sequences associated with published or patented SARS-CoV-1 or SARS-CoV-2 binding antibodies were mined from CoV-AbDab¹⁶, downloaded on 10 May 2020. A total of 80 non-redundant CDRH3s were identified (100% identity threshold). These sequences were then clustered alongside the representative CDRH3 sequence from each of our 777 convergent clones using CD-HIT³⁷, at an 80% sequence identity threshold (allowing at most a CDRH3 length mismatch of 1 AA). Cluster centres containing at least one CoV-AbDab CDRH3 and one convergent clone CDRH3 were further investigated.

### Public COVID-19 BCR Sequence Data Processing

The fourteen MiSeq “read 1” FASTQ datasets from the six SARS-CoV-2 patients analysed in Nielsen et al.¹⁴ were downloaded from the Sequence Read Archive³⁸. IgBlast³³ was used to identify heavy chain V, D, and J gene rearrangements and antibody regions. Unproductive sequences, sequences with out-of-frame V and J genes, and sequences missing the CDRH3 region were removed from the downstream analysis. Sequences with 100% amino acid and isotype matches were collapsed. To circumvent the disparity in collapsed dataset sizes between pairs of replicates, we selected the replicate with the highest number of sequences for downstream analysis.

### Convergent Clonotyping Matching to Public Repertoires

The public SARS-CoV-2-positive¹⁴ and healthy control BCR repertoires³⁹ were scanned for clonotype matches to our 777 convergent clonotype cluster centres. A BCR repertoire sequence was determined as a match if it had identical V and J genes, the same length CDRH3, and was within 1 AA mismatch per 10 CDRH3 AAs to a convergent clonotype representative sequence.

### Statistical Analysis and Graphing

Statistical analysis and plotting were performed using R⁴⁰. Plotting was performed using ggplot2⁴¹. Sequence logos were created using ggseqlogo⁴². Specific statistical tests used are detailed in the figure descriptions. Correlations of IGHV4-34 autoreactive motifs and convergent clonotypes was performed by manova in R.

### REFERENCES

1. Lu, H., Stratton, C. W. & Tang, Y. W. Outbreak of pneumonia of unknown etiology in Wuhan, China: The mystery and the miracle. Journal of Medical Virology vol. 92 401-402 (2020).
2. Huang, C. et al. Clinical features of patients infected with 2019 novel coronavirus in Wuhan, China. Lancet 395, 497-506 (2020).
3. Wan, Y., Shang, J., Graham, R., Baric, R. S. & Li, F. Receptor Recognition by the Novel Coronavirus from Wuhan: an Analysis Based on Decade-Long Structural Studies of SARS Coronavirus. J. Virol. 94, (2020).
4. Chen, N. et al. Epidemiological and clinical characteristics of 99 cases of 2019 novel coronavirus pneumonia in Wuhan, China: a descriptive study. Lancet 395, 507-513 (2020).
5. Mulangu, S. et al. A randomized, controlled trial of Ebola virus disease therapeutics. N. Engl. J. Med. 381, 2293-2303 (2019).
6. Gogtay, N. J. et al. Comparison of a Novel Human Rabies Monoclonal Antibody to Human Rabies Immunoglobulin for Postexposure Prophylaxis: A Phase 2/3, Randomized, Single-Blind, Noninferiority, Controlled Study. Clin. Infect. Dis. 66, 387-395 (2018).
7. Johnson, S. et al. Development of a Humanized Monoclonal Antibody (MEDI-493) with Potent In Vitro and In Vivo Activity against Respiratory Syncytial Virus. J. Infect. Dis. 176, 1215-1224 (1997).
8. Wang, S. F. et al. Antibody-dependent SARS coronavirus infection is mediated by antibodies against spike proteins. Biochem. Biophys. Res. Commun. 451, 208-214 (2014).
9. Tetro, J. A. Is COVID-19 receiving ADE from other coronaviruses? Microbes Infect. 22, 72-73 (2020).
10. Sharma, A. It is too soon to attribute ADE to COVID-19. Microbes and Infection (2020) doi:10.1016/j.micinf.2020.03.005.
11. Wang, Q. et al. Immunodominant SARS coronavirus epitopes in humans elicited both enhancing and neutralizing effects on infection in non-human primates. ACS Infect. Dis. 2, 361-376 (2016).
12. Brouwer, P. et al. Potent neutralizing antibodies from COVID-19 patients define multiple targets of vulnerability. bioRxiv (2020) doi:10.1101/2020.05.12.088716.
13. Andreano, E. et al. Identification of neutralizing human monoclonal antibodies from Italian Covid-19 convalescent patients. bioRxiv (2020) doi:10.1101/2020.05.05.078154.
14. Nielsen, S. et al. B cell clonal expansion and convergent antibody responses to SARS-CoV-2. Res. Sq. (2020) doi:10.21203/rs.3.rs-27220/v1.
15. Ghraichy, M. et al. Maturation of naïve and antigen-experienced B-cell receptor repertoires with age. bioRxiv (2019) doi:10.1101/609651.
16. Raybould, M. I. J., Kovaltsuk, A., Marks, C. & Deane, C. M. CoV-AbDab: the Coronavirus Antibody Database. bioRxiv (2020) doi:10.1101/2020.05.15.077313.
17. Pascual, V. et al. Nucleotide sequence analysis of the V regions of two IgM cold agglutinins: Evidence that the V(H)4-21 gene segment is responsible for the major cross-reactive idiotype. J. Immunol. 146, 4385-4391 (1991).
18. Schickel, J. N. et al. Self-reactive VH4-34-expressing IgG B cells recognizecommensal bacteria. J. Exp. Med. 214, 1991-2003 (2017).
19. Tipton, C. M. et al. Diversity, cellular origin and autoreactivity of antibody-secreting cell population expansions in acute systemic lupus erythematosus. Nat. Immunol. 16, 755-765 (2015).
20. Horns, F. et al. Lineage tracing of human B cells reveals the in vivo landscape of human antibody class switching. Elife 5, 1-20 (2016).
21. Parameswaran, P. et al. Convergent Antibody Signatures in Human Dengue. Cell Host Microbe 13, 691-700 (2013).
22. De Mattos-Arruda, L. et al. The Genomic and Immune Landscapes of Lethal Metastatic Breast Cancer. Cell Rep. 27, 2690-2708.e10 (2019).
23. Zhou, P. et al. A pneumonia outbreak associated with a new coronavirus of probable bat origin. Nature 579, 270-273 (2020).
24. Pinto, D. et al. Structural and functional analysis of a potent sarbecovirus neutralizing antibody. bioRxiv (2020) doi:10.1101/2020.04.07.023903.
25. Wec, A. Z. et al. Broad sarbecovirus neutralizing antibodies define a key site of vulnerability on the SARS-CoV-2 spike protein. bioRxiv (2020) doi:10.1101/2020.05.15.096511.
26. Grifoni, A. et al. Targets of T cell responses to SARS-CoV-2 coronavirus in humans with COVID-19 disease and unexposed individuals. Cell (2020) doi:10.1016/j.cell.2020.05.015.
27. Ng, K. et al. Pre-existing and de novo humoral immunity to SARS-CoV-2 in humans. BioRxiv (2020) doi:10.1101/2020.05.14.095414.
28. Duan, K. et al. Effectiveness of convalescent plasma therapy in severe COVID-19 patients. Proc. Natl. Acad. Sci. 117, 202004168 (2020).
29. van Dongen, J. J. M. et al. Design and standardization of PCR primers and protocols for detection of clonal immunoglobulin and T-cell receptor gene recombinations in suspect lymphoproliferations: report of the BIOMED-2 Concerted Action BMH4-CT98-3936. Leukemia 17, 2257-317 (2003).
30. Vander Heiden, J. A. et al. pRESTO: a toolkit for processing high-throughput sequencing raw reads of lymphocyte receptor repertoires. Bioinformatics 30, 1930-2 (2014).
31. Gupta, N. T. et al. Change-O: A toolkit for analyzing large-scale B cell immunoglobulin repertoire sequencing data. Bioinformatics 31, 3356-3358 (2015).
32. Edgar, R. C. Search and clustering orders of magnitude faster than BLAST. Bioinformatics 26, 2460-2461 (2010).
33. Ye, J., Ma, N., Madden, T. L. & Ostell, J. M. IgBLAST: an immunoglobulin variable domain sequence analysis tool. Nucleic Acids Res. 41, W34-40 (2013).
34. Galson, J. D. et al. BCR repertoire sequencing: different patterns of B cell activation after two Meningococcal vaccines. Immunol. Cell Biol. 93, 885-95 (2015).
35. Vander Heiden, J. A. & Gupta, N. alakazam: Immunoglobulin Clonal Lineage and Diversity Analysis. R Packag. version 0.2.0 (2015).
36. Bolotin, D. A. et al. MiXCR: software for comprehensive adaptive immunity profiling. Nat. Methods 12, 380-381 (2015).
37. Fu, L., Niu, B., Zhu, Z., Wu, S. & Li, W. CD-HIT: Accelerated for clustering the next-generation sequencing data. Bioinformatics 28, 3150-3152 (2012).
38. Leinonen, R., Sugawara, H. & Shumway, M. The sequence read archive. Nucleic Acids Res. 39, (2011).
39. Briney, B., Inderbitzin, A., Joyce, C. & Burton, D. R. Commonality despite exceptional diversity in the baseline human antibody repertoire. Nature 566, 393-397 (2019).
40. Team, R. D. C. R: A language and environment for statistical computing. R Found. Stat. Comput. Vienna, Austria (2008).
41. Wickham, H. ggplot2: Elegant Graphics for Data Analysis. (Springer; 1st ed. 2009. Corr. 3rd printing 2010 edition, 2009).
42. Wagih, O. Ggseqlogo: A versatile R package for drawing sequence logos. Bioinformatics 33, 3645-3647 (2017).
Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. All patents and patent applications mentioned throughout the specification of the present invention are herein incorporated in their entirety by reference. The invention embraces all combinations of preferred and more preferred groups and suitable and more suitable groups and embodiments of groups recited above.

1. A polypeptide comprising:
a CDRH1 sequence comprising or consisting of a sequence sharing 80% or greater sequence identity with a CDRH1 sequence as shown in Table 1 and/or
a CDRH2 sequence comprising or consisting of a sequence sharing 80% or greater sequence identity with a CDRH2 sequence as shown in Table 1 and/or
a CDRH3 sequence comprising or consisting of a sequence sharing 80% or greater sequence identity with a CDRH3 sequence as shown in Table 1.

2. The polypeptide according to claim 1 wherein the polypeptide comprises
a CDRH1 sequence comprising or consisting of a sequence sharing 90% or greater sequence identity with a CDRH1 sequence as shown in Table 1 and/or
a CDRH2 sequence comprising or consisting of a sequence sharing 90% or greater sequence identity with a CDRH2 sequence as shown in Table 1 and/or
a CDRH3 sequence comprising or consisting of a sequence sharing 90% or greater sequence identity with a CDRH3 sequence as shown in Table 1.

3. The polypeptide according to claim 2 wherein the polypeptide comprises
a CDRH1 sequence comprising or consisting of a CDRH1 sequence as shown in Table 1 and/or
a CDRH2 sequence comprising or consisting of a CDRH2 sequence as shown in Table 1 and/or
a CDRH3 sequence comprising or consisting of a CDRH3 sequence as shown in Table 1.

4. The polypeptide according to claim 3 wherein the polypeptide comprises
a CDRH1 sequence comprising or consisting of a CDRH1 sequence as shown in Table 1 and
a CDRH2 sequence comprising or consisting of a CDRH2 sequence as shown in Table 1 and
a CDRH3 sequence comprising or consisting of a CDRH3 sequence as shown in Table 1.

5. The polypeptide according to any preceding claim, wherein the polypeptide comprises
a FWRH1 sequence comprising or consisting of a sequence sharing 80% or greater sequence identity with a FWRH1 sequence as shown in Table 1 and/or
a FWRH2 sequence comprising or consisting of a sequence sharing 80% or greater sequence identity with a FWRH2 sequence as shown in Table 1 and/or
a FWRH3 sequence comprising or consisting of a sequence sharing 80% or greater sequence identity with a FWRH3 sequence as shown in Table 1 and/or
a FWRH4 sequence comprising or consisting of a sequence sharing 80% or greater sequence identity with a FWRH4 sequence as shown in Table 1.

6. A polypeptide comprising:
a FWRH1 sequence comprising or consisting of a sequence sharing 80% or greater sequence identity with a FWRH1 sequence as shown in Table 1 and/or
a FWRH2 sequence comprising or consisting of a sequence sharing 80% or greater sequence identity with a FWRH2 sequence as shown in Table 1 and/or
a FWRH3 sequence comprising or consisting of a sequence sharing 80% or greater sequence identity with a FWRH3 sequence as shown in Table 1 and/or
a FWRH4 sequence comprising or consisting of a sequence sharing 80% or greater sequence identity with a FWRH4 sequence as shown in Table 1.

7. The polypeptide according to either claim 5 or 6, wherein the polypeptide comprises
a FWRH1 sequence comprising or consisting of a sequence sharing 90% or greater sequence identity with a FWRH1 sequence as shown in Table 1 and/or
a FWRH2 sequence comprising or consisting of a sequence sharing 90% or greater sequence identity with a FWRH2 sequence as shown in Table 1 and/or
a FWRH3 sequence comprising or consisting of a sequence sharing 90% or greater sequence identity with a FWRH3 sequence as shown in Table 1 and/or
a FWRH4 sequence comprising or consisting of a sequence sharing 90% or greater sequence identity with a FWRH4 sequence as shown in Table 1.

8. The polypeptide according to claim 7, wherein the polypeptide comprises
a FWRH1 sequence comprising or consisting of a FWRH1 sequence as shown in Table 1 and/or
a FWRH2 sequence comprising or consisting of a FWRH2 sequence as shown in Table 1 and/or
a FWRH3 sequence comprising or consisting of a FWRH3 sequence as shown in Table 1 and/or
a FWRH4 sequence comprising or consisting of a FWRH4 sequence as shown in Table 1.

9. The polypeptide according to claim 8, wherein the polypeptide comprises
a FWRH1 sequence comprising or consisting of a FWRH1 sequence as shown in Table 1 and
a FWRH2 sequence comprising or consisting of a FWRH2 sequence as shown in Table 1 and
a FWRH3 sequence comprising or consisting of a FWRH3 sequence as shown in Table 1 and
a FWRH4 sequence comprising or consisting of a FWRH4 sequence as shown in Table 1.

10. The polypeptide according to any preceding claim, wherein the polypeptide comprises three complementarity determining regions (CDRH1-CDRH3).

11. The polypeptide according to any preceding claim, wherein the polypeptide comprises four framework regions (FWRH1-FWRH4).

12. A polypeptide comprising or consisting of a sequence sharing 80% or greater sequence identity with any immunoglobulin heavy chain variable domain (VH) sequence as shown in Table 1 or Table 2.

13. The polypeptide according to claim 12, wherein the polypeptide comprises or consists of a sequence sharing 90% or greater sequence identity with any immunoglobulin heavy chain variable domain (VH) sequence as shown in Table 1 or Table 2.

14. The polypeptide according to either claim 12 or 13 wherein the polypeptide comprises or consists of an immunoglobulin heavy chain variable domain (VH) sequence as shown in Table 1 or Table 2.

15. The polypeptide according to any preceding claim which is paired with a cognate light chain polypeptide.

16. The polypeptide according to any preceding claim wherein the polypeptide is an antibody.

17. The polypeptide according to claim 15 wherein the antibody belongs to the isotype subclass IGHA1, IGHA2, IGHG1, IGHG2, IGHG3 or IGHG4.

18. The polypeptide according to any one of claims 1 to 14 wherein the polypeptide is an antibody fragment, such as a F(ab′)₂, an Fd, an Fv, an scFv, a VH, or a VHH.

19. The polypeptide according to any preceding claim, wherein the polypeptide binds to the spike protein (S protein) of SARS-CoV-2.

20. The polypeptide according to claim 19 wherein the polypeptide binds to the S1 or S2 domain of the spike protein (S protein), such as the S1 domain of the spike protein (S1 protein).

21. Polypeptide according to any preceding claims which binds to SARS-Cov2 viral proteins other than the spike protein.

22. Polypeptide according to any preceding claims which binds to SARS-CoV2 infected human cells.

23. Polypeptide according to any preceding claim which binds to a human protein to reduce viral load, increase viral neutralisation or beneficially modify immune responses occurring as a consequence of virus infection.

24. A pharmaceutical composition comprising the polypeptide according to any preceding claim and one or more pharmaceutically acceptable diluents or carriers.

25. The pharmaceutical composition according to claim 24 further comprising up to 3 polypeptides that may bind to different epitopes, in various combination ratios according to any preceding claim.

26. The pharmaceutical composition according to either claim 24 or 25 comprising at least one further active agent such as an anti-viral or anti-inflammatory agent.

27. The polypeptide or pharmaceutical composition according to any preceding claims, for use in suppressing or treating a disease or disorder mediated by infection of SARS-CoV-2, such as COVID-19, or for providing prophylaxis to a subject at risk of infection of SARS-CoV-2, such as COVID-19.

28. A method of suppressing or treating a disease or disorder mediated by infection of SARS-CoV-2, such as COVID-19 or for providing prophylaxis to a subject at risk of infection of SARS-CoV-2, such as COVID-19, comprising administering to a person in need thereof a therapeutically effective amount of the polypeptide or pharmaceutical composition according to any preceding claims.

29. A polypeptide or pharmaceutical composition according to any preceding claims, for providing treatment or prophylaxis of an infection mediated by other/new forms of coronavirus.

30. One or more polypeptides according to any preceding claims, for use in diagnosis and/or prediction of outcome of SARS-CoV-2 infection.

31. A polypeptide or pharmaceutical composition according to any preceding claims, for use in medical equipment in order to prevent or reduce the risk of infection (e.g., mask or air filter).

32. A polynucleotide encoding the polypeptide according to any one of claims 1 to 23.

33. A vector comprising the polynucleotide according to claim 32.
